(19) United States
(12) Patent Application Publication
(10) Pub. No.: US 2016/0202242 A1
(43) Pub. Date: Jul. 14, 2016

(54) CELL DEATH-INDUCING AGENT, CELL GROWTH-INHIBITING AGENT, AND PHARMACEUTICAL COMPOSITION FOR TREATMENT OF DISEASE CAUSED BY ABNORMAL CELL GROWTH

(71) Applicant: Nitto Denko Corporation, Osaka (JP)

(72) Inventors: Hiroyuki TANAKA, Osaka (JP); Kenjirou MINOMI, Osaka (JP); Yoshiro NIITSU, Hokkaido (JP)

(21) Appl. No.: 14/980,055

(22) Filed: Dec. 28, 2015

Related U.S. Application Data
(63) Continuation-in-part of application No. 14/793,212, filed on Jul. 7, 2015.

(30) Foreign Application Priority Data
Dec. 26, 2014 (JP) 2014-266198
Jul. 6, 2015 (JP) 2015-135494
Dec. 18, 2015 (JP) 2015-247725

(51) Int. Cl. G01N 33/50; A61K 31/713

(52) U.S. Cl. CPC G01N 33/5011; A61K 31/713

(57) ABSTRACT

It is intended to induce cell death and inhibit cell growth for cancer cells.

The agents of the present invention comprise, as active ingredients, a drug inhibiting GST-π and a drug inhibiting a homeostasis-related protein that exhibits synthetic lethality when inhibited together with GST-π.

### TECHNICAL FIELD

The present invention relates to a cell death-inducing agent and a cell growth-inhibiting agent for a cancer cell, and a pharmaceutical composition for the treatment of a disease caused by abnormal cell growth and further relates to a method for screening for a cell death-inducing agent and/or a cell growth-inhibiting agent.

### BACKGROUND ART

Typical examples of diseases caused by abnormal cell growth can include cancers. Cancers are diseases in which cells grow in an uncontrolled manner due to mutations, epigenetic abnormalities, etc., in genes. A large number of gene abnormalities in cancers have already been reported (e.g., Futreal et al., Nat Rev Cancer. 2004; 4 (3): 177-83), most of which are considered to have some relation to signal transduction involved in cell growth, differentiation, or survival. Moreover, such gene abnormalities cause abnormal signal transduction in cells constituted by normal molecules. This may bring about the activation or deactivation of a particular signal cascade and eventually become partly responsible for the abnormal growth of the cells.

Although a principal objective of cancer treatment in early times was to inhibit cell growth itself, such treatment physiologically inhibited even the growth of normal cells and therefore involved adverse reactions such as alopecia, gastrointestinal disturbances, and myelosuppression. Accordingly, therapeutic drugs for cancers based on novel ideas such as molecular target drugs, which target cancer-specific gene abnormalities or abnormal signal transduction, are under development in order to prevent such adverse reactions.

A cancer is thought to occur by the accumulation of abnormalities in various cancer genes, tumor suppressor genes, DNA repair enzyme genes, and the like in the same cell. RAS gene, FOS gene, MYC gene, and BCL-2 gene, etc., are known as the cancer genes. Among cancer-specific gene abnormalities, a mutation is found in KRAS gene in approximately 95% pancreatic cancer, approximately 45% colorectal cancer, and many other cancers with high frequency. The KRAS protein is a G protein that is localized to the inner side of a cell membrane. RAS including KRAS forms a cascade where RAS activates RAF such as C-RAF or B-RAF, and subsequently, this RAF activates MEK, which then activates MAPK. When a point mutation takes place in KRAS, GTPase activity is reduced so that GTP-bound active forms are maintained to thereby constitutively sustain signals to downstream pathway, resulting in abnormal cell growth. As typified by the KRAS gene, the cancer genes cause abnormal cell growth which in turn progresses to the malignant transformation of the cell and eventually a cancer as a disease.

Incidentally, glutathione-S-transferase (GST), an enzyme catalyzing glutathione conjugation, is known as an enzyme that converts a substance such as a drug to a water-soluble substance through coupling with glutathione (GSH). GST is typically classified, on the basis of amino acid sequences, into 6 types of isozymes: α, μ, ω, π, θ, and ζ. Among them, particularly, the expression of GST-π (glutathione S-transferase pi, also called GSTP1) is increased in various cancer cells. The possibility has been pointed out that this is partly responsible for resistance to some anticancer agents. In fact, it is known that when an antisense DNA against GST-π or a GST-π inhibitor is allowed to act on a GST-π-overexpressing cancer cell line that exhibits drug resistance, the drug resistance is inhibited (Takahashi and Niitsu, Gan To Kagaku Ryoho. 1994; 21 (7): 945-51; Ban et al., Cancer Res. 1996; 56 (15): 3577-82; and Nakajima et al., J Pharmacol Exp Ther. 2003; 306 (3): 861-9). In addition, a recent report has showed that when an siRNA against GST-π is allowed to act on a GST-π-overexpressing androgen-independent prostate cancer cell line, its growth is inhibited so that apoptosis is increased (Hokaiwado et al., Carcinogenesis. 2008; 29 (6): 1134-8).

It is also known that GST-π forms a complex with c-Jun N-terminal kinase (JNK) to inhibit JNK activity (Adler et.al, EMBO J. 1999, 18, 1321-1334). It is further known that GST-π participates in the S-glutathionylation of proteins associated with the stress response of cells (Townsend, et.al, J. Biol. Chem. 2009, 284, 436-445). In addition, it is known that GST-π contributes to a protective effect against cell death induced by reactive oxygen species (ROS) (Yin et.al, Cancer Res. 2000 60, 4053-4057). Thus, it can be understood that among the GST isozymes, GST-π has various features and functions.

It has been reported that when an siRNA against GST-π is allowed to act on a cancer cell line having a mutation in KRAS, the activation of Akt is inhibited so that autophagy is enhanced whereas apoptosis is moderately induced (Nishita et al., AACR 102nd Annual Meeting, Abstract No. 1065). WO2012/176282 discloses that use of a drug inhibiting GST-π and an autophagy inhibitor such as 3-methyladenine as active ingredients can induce the apoptosis of cancer cells. Furthermore, WO2014/098210 discloses that: when the expression of GST-π and the expression of Akt or the like are inhibited at the same time, cell growth is inhibited to induce cell death; and autophagy induced by the inhibition of GST-π expression is significantly inhibited by inhibiting the expression of GST-π and the expression Akt or the like at the same time.

However, much still remains unknown about the relation of the expression of GST-π in cancer cells to cell growth or cell death, the role of GST-π in signal transduction, etc.

### SUMMARY OF INVENTION

### Object to be Attained by the Invention

Thus, an object of the present invention is to provide an agent having a cell death-inducing effect and/or a cell growth-inhibiting effect on a cancer cell, to provide a pharmaceutical composition for the treatment of a disease caused by abnormal cell growth, and to provide a method for screening for a cell death-inducing agent and/or a cell growth-inhibiting agent.

### Means for Attaining the Object

The present inventors have conducted diligent studies in light of the object mentioned above and consequently completed the present invention by finding that a homeostasis-related protein that exhibits synthetic lethality when inhibited together with GST-π is inhibited along with the inhibition of GST-π in a cancer cell, whereby cell death is more strongly induced and cell growth is more strongly inhibited as compared with the case where either of them is inhibited. The present invention encompasses the following:

(1) A cell death-inducing agent for a cancer cell comprising, as active ingredients, a drug inhibiting GST-π and a drug inhibiting a homeostasis-related protein that exhibits synthetic lethality when inhibited together with GST-π.

(2) A cell growth-inhibiting agent for a cancer cell comprising, as active ingredients, a drug inhibiting GST-π and a drug inhibiting a homeostasis-related protein that exhibits synthetic lethality when inhibited together with GST-π.

(3) The agent according to (1) or (2), wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

(4) The agent according to (3), wherein the cell cycle-regulating protein that exhibits synthetic lethality along with the inhibition of GST-π is at least one cell cycle-regulating protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

(5) The agent according to (3), wherein the cell cycle-regulating protein that exhibits synthetic lethality along with the inhibition of GST-π is at least one protein selected from the group consisting of p21, RNPC1, CCNL1, MCM8, CCNB3, and MCMDC1.

(6) The agent according to (3), wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

(7) The agent according to (3), wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.

(8) The agent according to (1) or (2), wherein the drug is a substance selected from the group consisting of an RNAi molecule, a ribozyme, an antisense nucleic acid, a DNA/RNA chimeric polynucleotide, and a vector for expressing at least one of them.

(9) The agent according to (1) or (2), wherein the drug inhibiting a homeostasis-related protein is a compound that acts on the homeostasis-related protein.

(10) The agent according to (1), wherein the agent induces apoptosis.

(11) The agent according to (1) or (2), wherein the cancer cell is a cancer cell highly expressing GST-π.

(12) A pharmaceutical composition for the treatment of a disease caused by abnormal cell growth, comprising an agent according to any of (1) to (11).

(13) The pharmaceutical composition according to (12), wherein the disease is a cancer.

(14) The pharmaceutical composition according to (13), wherein the cancer is a cancer highly expressing GST-π.

(15) A method for screening for a cell death-inducing agent and/or a cell growth-inhibiting agent for a cancer cell that is used together with a drug inhibiting GST-π, comprising a step of selecting a drug inhibiting a homeostasis-related protein that exhibits synthetic lethality when inhibited together with GST-π.

(16) The screening method according to (14), wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

(17) The screening method according to (16), wherein the cell cycle-regulating protein that exhibits synthetic lethality along with the inhibition of GST-π is at least one protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

(18) The screening method according to (16), wherein the cell cycle-regulating protein that exhibits synthetic lethality along with the inhibition of GST-π is at least one protein selected from the group consisting of p21, RNPC1, CCNL1, MCM8, CCNB3, and MCMDC1.

(19) The screening method according to (16), wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

(20) The screening method according to (16), wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.

(21) The screening method according to any of (15) to (20), comprising the steps of: contacting a test substance with a cancer cell; measuring the expression level of the homeostasis-related protein in the cell; and selecting the test substance as a drug inhibiting the homeostasis-related protein when the expression level is decreased compared with that measured in the absence of the test substance.

(22) A method for screening for a cell death-inducing agent and/or cell growth-inhibiting agent for a cancer cell that is used together with a drug inhibiting a homeostasis-related protein that exhibits synthetic lethality when inhibited together with GST-π, comprising a step of selecting a drug inhibiting GST-π.

(23) The screening method according to (22), wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

(24) The screening method according to (22), wherein the cell cycle-regulating protein that exhibits synthetic lethality along with the inhibition of GST-π is at least one protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

(25) The screening method according to (23), wherein the cell cycle-regulating protein that exhibits synthetic lethality along with the inhibition of GST-π is at least one protein selected from the group consisting of p21, RNPC1, CCNL1, MCM8, CCNB3, and MCMDC1.

(26) The screening method according to (23), wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

(27) The screening method according to (23), wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.

(28) The screening method according to any of (22) to (27), comprising the steps of: contacting a test substance with a cancer cell; measuring the expression level of GST-π in the cell; and selecting the test substance as a drug inhibiting GST-π when the expression level is decreased compared with that measured in the absence of the test substance.

(29) A method for screening for a cell death-inducing agent and/or a cell growth-inhibiting agent, comprising a step of selecting a drug inhibiting GST-π and homeostasis-related protein that exhibits synthetic lethality when inhibited together with GST-π.

(30) The screening method according to (29), wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

(31) The screening method according to (30), wherein the cell cycle-regulating protein that exhibits synthetic lethality along with the inhibition of GST-π is at least one cell cycle-regulating protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

(32) The screening method according to (30), wherein the cell cycle-regulating protein that exhibits synthetic lethality along with the inhibition of GST-π is at least one protein selected from the group consisting of p21, RNPC1, CCNL1, MCM8, CCNB3, and MCMDC1.

(33) The screening method according to (30), wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

(34) The screening method according to (30), wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.

(35) The screening method according to any of (29) to (34), comprising the steps of: contacting a test substance with a cancer cell; measuring the expression level of GST-π and the expression level of the homeostasis-related protein that exhibits synthetic lethality when inhibited together with GST-π, in the cell; and selecting the test substance as a drug inhibiting GST-π and the homeostasis-related protein that exhibits synthetic lethality when inhibited together with GST-π when the expression level of GST-π and the expression level of the homeostasis-related protein that exhibits synthetic lethality when inhibited together with GST-π are both decreased compared with those measured in the absence of the test substance.

This description includes part or all of the contents as disclosed in the description and/or drawings of Japanese Patent Application No. 2014-266198, Japanese Patent Application No. 2015-135494 and Japanese Patent Application No. 2015-247725, which are priority documents of the present application.

### Effects of the Invention

The cell death-inducing agent according to the present invention can very strongly induce cell death for a cancer cell. Accordingly, the cell death-inducing agent according to the present invention can exert very high efficacy as a pharmaceutical composition for the treatment of a disease caused by the abnormal growth of the cancer cell.

Moreover, the cell growth-inhibiting agent according to the present invention can very strongly inhibit cell growth for a cancer cell. Accordingly, the cell growth-inhibiting agent according to the present invention can exert very high efficacy as a pharmaceutical composition for the treatment of a disease caused by the abnormal growth of the cancer cell.

Furthermore, the screening method according to the present invention can select a drug that very strongly induces cell death and/or inhibits cell growth for a cancer cell.

### BRIEF DESCRIPTION OF THE DRAWINGS

FIG. 1 is a characteristic diagram showing results of assaying GST-π mRNA and p21 mRNA in cells expressing mutated KRAS when an siRNA inhibiting the expression of GST-π and/or an siRNA inhibiting the expression of p21 was allowed to act thereon.

FIG. 2 is a characteristic diagram showing results of quantifying over time p21 mRNA when GST-π and p21 were both knocked down.

FIG. 3 is a characteristic diagram showing results of measuring the number of cells when GST-π and p21 were both knocked down.

FIG. 4 is a characteristic diagram showing results of measuring the number of cells when GST-π and p21 were both knocked down three times.

FIG. 5 is a characteristic diagram showing results of measuring the number of cells when GST-π and p21 were both knocked down three times.

FIG. 6 is a photograph taken for the phase difference image of A549 cells when GST-π and p21 were both knocked down three times.

FIG. 7 is a photograph taken for the phase difference image of MIA PaCa-2 cells when GST-π and p21 were both knocked down three times.

FIG. 8 is a photograph taken for the phase difference image of PANC-1 cells when GST-π and p21 were both knocked down three times.

FIG. 9 is a photograph taken for the phase difference image of HCT116 cells when GST-π and p21 were both knocked down three times.

FIG. 10 is a photograph taken for the phase difference image of M7609 cells when GST-π was knocked down three times and (3-galactosidase staining was carried out.

FIG. 11 is a characteristic diagram showing results of quantifying the expression of PUMA gene when GST-π and p21 were both knocked down.

FIG. 12 is a characteristic diagram showing results of comparing relative survival rates when GST-π and a candidate protein (cell cycle-regulating protein) exhibiting synthetic lethality were knocked down each alone and when GST-π and the candidate protein were both knocked down.

FIG. 13 is a characteristic diagram showing results of comparing relative survival rates when GST-π and a candidate protein (anti-apoptosis-related protein) exhibiting synthetic lethality were knocked down each alone and when GST-π and the candidate protein were both knocked down.

FIG. 14 is a characteristic diagram showing results of comparing relative survival rates when GST-π and a candidate protein (PI3K signaling pathway-related protein) exhibiting synthetic lethality were knocked down each alone and when GST-π and the candidate protein were both knocked down.

FIG. 15 is a characteristic diagram showing results of comparing relative survival rates when GST-π and MYLK were knocked down each alone and when GST-π and MYLK were both knocked down.

### DESCRIPTION OF THE PREFERRED EMBODIMENTS

The cell death-inducing agent and the cell growth-inhibiting agent according to the present invention comprise, as active ingredients, a drug inhibiting GST-π and a drug inhibiting a homeostasis-related protein that exhibits synthetic lethality when inhibited together with GST-π. The cell death-inducing agent and the cell growth-inhibiting agent according to the present invention exhibit a cell death-inducing effect and a cell growth-inhibiting effect on a cancer cell. In this context, the cancer cell is a cell that exhibits abnormal growth attributed to genes (cancer-related genes).

Of the cancer-related genes, examples of cancer genes can include KRAS gene, FOS gene, MYC gene, BCL-2 gene, and SIS gene. Also, of the cancer-related genes, examples of tumor suppressor genes can include RB gene, p53 gene, BRCA1 gene, NF1 gene, and p73 gene. However, the cancer cell is not limited to cancer cells in which these specific cancer-related genes are involved, and the agents of the present invention can be applied to a wide range of cells that exhibit abnormal cell growth.

Particularly, it is preferable that the cell death-inducing agent and the cell growth-inhibiting agent according to the present invention should be applied to a cancer cell highly expressing GST-π, among the cancer cells. In this context, the cancer cell highly expressing GST-π means a cell having a significantly higher expression level of GST-π than that of a normal cell, among the cells that exhibit abnormal cell growth (so-called cancer cells). The expression level of GST-π can be measured according to a standard method such as RT-PCR or microarrays.

In many cases, one example of the cancer cell highly expressing GST-π can include a cancer cell expressing mutated KRAS. Specifically, it is preferable that the cell death-inducing agent and the cell growth-inhibiting agent according to the present invention should be applied to the cancer cell expressing mutated KRAS.

The mutated KRAS means a protein having an amino acid sequence in which mutation(s) such as deletion, substitution, addition, and/or insertion is introduced in the amino acid sequence of wild-type KRAS. In this context, the mutation in the mutated KRAS is a so-called gain of function mutation. Specifically, in the cell expressing mutated KRAS, for example, GTPase activity is reduced due to the mutation so that GTP-bound active forms are maintained to thereby constitutively sustain signals to downstream pathway, resulting in abnormal cell growth as compared with the cell expressing wild-type KRAS. Examples of a gene encoding the mutated KRAS include a gene having a mutation at at least one of codon 12, codon 13, and codon 61 in the wild-type KRAS gene. Particularly, mutations at codons 12 and 13 are preferable for the mutated KRAS. Specific examples thereof include mutations by which glycine encoded by codon 12 of the KRAS gene is replaced with serine, aspartic acid, valine, cysteine, alanine, or arginine, and mutations by which glycine encoded by codon 13 of the KRAS gene is changed to aspartic acid.

As used herein, GST-π refers to an enzyme that is encoded by GSTP1 gene and catalyzes glutathione conjugation. GST-π is present in various animals including humans, and its sequence information is also publicly known (e.g., human: NM 000852 (NP 000843), rat: NM 012577 (NP 036709), mouse: NM 013541 (NP 038569), etc.; the numbers represent accession numbers of the NCBI database, and the nucleotide sequence and the amino acid sequence are indicated outside and inside the parentheses, respectively). As one example, the nucleotide sequence of the coding region of the human GST-π gene registered in the database is shown in SEQ ID NO: 1, and the amino acid sequence of the human GST-π protein encoded by this human GST-π gene is shown in SEQ ID NO: 2.

As used herein, the homeostasis-related protein that exhibits synthetic lethality when inhibited together with GST-π is a protein that results in a significantly high death rate of a cancer cell when inhibited together with GST-π as compared with the death rate of the cancer cell brought about by the inhibition of GST-π alone. This protein has the function of participating in cell homeostasis. In this context, the synthetic lethality means a phenomenon in which lethality is exerted or significantly enhanced for a cell or an individual by a combination of defects of a plurality of genes, though only one of the gene defects leads to no or low lethality. Particularly, in the present specification, the synthetic lethality means lethality for a cancer cell.

In the present specification, examples of the homeostasis-related protein that exhibits synthetic lethality when inhibited together with GST-π can include a cell cycle-regulating protein, an anti-apoptosis-related protein, and a PI3K signaling pathway-related protein. The cell cycle-regulating protein is a protein having the function of regulating cell cycle. The anti-apoptosis-related protein is a protein having the function of suppressively participating in apoptosis. The PI3K signaling pathway-related protein is a protein, except for Akt1, included in proteins involved in the PI3K/AKT signaling pathway.

Also, the protein having the function of regulating cell cycle is meant to include every protein involved in cell cycle consisting of the G1 phase (resting stage before DNA replication), the S phase (DNA synthesis stage), G2 (resting stage before cell division), and the M phase (cell division stage). More specifically, examples of the regulation of cell cycle can include each event of the regulation of the mechanism of promoting the G1 phase the S phase the G2 phase the M phase in order, the regulation of progression at the G1 phase to the S phase, and the regulation of progression at the G2 phase to the M phase. Thus, the cell cycle-regulating protein can be, for example, a protein that participates in the progression of these events in cell cycle and a protein that positively or negatively regulates these events. Further specifically, examples of the cell cycle-regulating protein include cyclin-dependent kinases (CDKs) essential for the initiation of the S phase and the M phase. The activity of the cyclin-dependent kinases is positively regulated by the binding of cyclins. Also, the activity of the cyclin-dependent kinases is negatively regulated by cyclin-dependent kinase inhibitors (CKIs) such as p21 (CIP1/WAF1) and tyrosine kinases. Thus, these proteins regulating the activity of the cyclin-dependent kinases, i.e., cyclins, cyclin-dependent kinase inhibitors (such as p21), and tyrosine kinases, are also included in the cell cycle-regulating protein.

Specifically, examples of the cell cycle-regulating protein that exhibits synthetic lethality when inhibited together with GST-π can include at least one cell cycle-regulating protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK. Of these 15 types of cell cycle-regulating proteins, one type of cell cycle-regulating protein may be inhibited together with GST-π, or two or more types of cell cycle-regulating proteins may be inhibited together with GST-π.

Particularly, it is preferable for the cell cycle-regulating protein that at least one cell cycle-regulating protein selected from the group consisting of p21, RNPC1, CCNL1, MCM8, CCNB3, and MCMDC1 should be inhibited together with GST-π. These 6 types of cell cycle-regulating proteins have a relatively low rate of cell growth inhibition when inhibited each alone, and exhibit a remarkably high cell growth-inhibiting effect only when inhibited together with GST-π. That is, it can be said that a drug inhibiting any of these 6 types of cell cycle-regulating proteins is excellent in safety by itself. Thus, it is preferable that the cell cycle-regulating protein that exhibits synthetic lethality when inhibited together with GST-π should be selected from these 6 types of cell cycle-regulating proteins.

p21 is a cell cycle-regulating protein that is encoded by CDKN1A gene and belongs to the CIP/KIP family. This protein has the function of inhibiting cell cycle progression at the G1 phase and the G2/M phase by inhibiting the effect of a cyclin-CDK complex through binding to the complex. Specifically, the p21 gene undergoes activation by p53 (one of tumor suppressor genes). It has been reported that upon activation of p53 due to DNA damage or the like, p53 activates p21 so that the cell cycle is arrested at the G1 phase and the G2/M phase. In addition, p21 also has the function of inhibiting apoptosis and has been reported to have the effect of protecting a cell from apoptosis induced by a chemotherapeutic agent or the like in in vitro and animal experiments (Gartel and Tyner, 2002; and Abbs and Dutta, 2009). p21 is present in various animals including humans, and its sequence information is also publicly known (e.g., human: NM 000389.4, NM 078467.2, NM 001291549.1, NM 001220778.1, NM 001220777.1 (NP 001207707.1, NP 001278478.1, NP 001207706.1, NP 510867.1, NP 000380.1), etc.; the numbers represent accession numbers of the NCBI database, and the nucleotide sequence and the amino acid sequence are indicated outside and inside the parentheses, respectively). As one example, the nucleotide sequence of the human CDKN1A gene registered in the database as NM_000389.4 is shown in SEQ ID NO: 3, and the amino acid sequence of the human p21 protein encoded by this human CDKN1A gene is shown in SEQ ID NO: 4. In the present specification, p21 is not limited to a protein consisting of the amino acid sequence of SEQ ID NO: 4 encoded by the nucleotide sequence of SEQ ID NO: 3. As for p21, sequence information has been registered with a plurality of accession numbers as mentioned above, and a plurality of transcript variants are present. The nucleotide sequence of SEQ ID NO: 3 represents the nucleotide sequence of one of these transcript variants.

RNPC1 is an RNA-binding protein encoded by RNPC1 gene and refers to a protein that is targeted by p53. RNPC1 is present in various animals including humans, and its sequence information is also publicly known (e.g., human: NM_017495.5, NM_183425.2, NM_001291780.1, XM_005260446.1 (XP_005260503.1, NP_059965.2, NP_906270.1, NP_001278709.1), etc.; the numbers represent accession numbers of the NCBI database, and the basic acid sequence and the amino acid sequence are indicated outside and inside the parentheses, respectively). As one example, the nucleotide sequence of the human RNPC1 gene registered in the database as NM_017495.5 is shown in SEQ ID NO: 5, and the amino acid sequence of the human RNPC1 protein encoded by this human RNPC1 gene is shown in SEQ ID NO: 6. In the present specification, RNPC1 is not limited to a protein consisting of the amino acid sequence of SEQ ID NO: 6 encoded by the nucleotide sequence of SEQ ID NO: 5. As for RNPC1, sequence information has been registered with a plurality of accession numbers as mentioned above, and a plurality of transcript variants are present. The nucleotide sequence of SEQ ID NO: 5 represents the nucleotide sequence of one of these transcript variants.

CCNL1 refers to cyclin-L1 encoded by CCNL1 gene. CCNL1 is present in various animals including humans, and its sequence information is also publicly known (e.g., human: NM_020307.2, XM_005247647.2, XM_005247648.1, XM_005247649.1, XM_005247650.1, XM_005247651.1, XM_006713710.1, XM_006713711.1 (XP_005247704.1, XP_005247705.1, XP_005247706.1, XP_005247707.1, XP_005247708.1, XP_006713773.1, NP_064703.1), etc.; the numbers represent accession numbers of the NCBI database, and the nucleotide sequence and the amino acid sequence are indicated outside and inside the parentheses, respectively). As one example, the nucleotide sequence of the human CCNL1 gene registered in the database as NM_020307.2 is shown in SEQ ID NO: 7, and the amino acid sequence of the human CCNL1 protein encoded by this human CCNL1 gene is shown in SEQ ID NO: 8. In the present specification, CCNL1 is not limited to a protein consisting of the amino acid sequence of SEQ ID NO: 8 encoded by the nucleotide sequence of SEQ ID NO: 7. As for CCNL1, sequence information has been registered with a plurality of accession numbers as mentioned above, and a plurality of transcript variants are present. The nucleotide sequence of SEQ ID NO: 7 represents the nucleotide sequence of one of these transcript variants.

MCM8 refers to mini-chromosome maintenance 8 encoded by MCM8 gene. MCM8 is present in various animals including humans, and its sequence information is also publicly known (e.g., human: NM_032485.5, NM_182802.2, NM_001281520.1, NM_001281521.1, NM_001281522.1, XM_005260859.1 (XP_005260916.1, NP_115874.3, NP_001268449.1, NP_877954.1, NP_001268450.1, NP_001268451.1), etc.; the numbers represent accession numbers of the NCBI database, and the nucleotide sequence and the amino acid sequence are indicated outside and inside the parentheses, respectively). As one example, the nucleotide sequence of the human MCM8 gene registered in the database as NM_032485.5 is shown in SEQ ID NO: 9, and the amino acid sequence of the human MCM8 protein encoded by this human MCM8 gene is shown in SEQ ID NO: 10. In the present specification, MCM8 is not limited to a protein consisting of the amino acid sequence of SEQ ID NO: 10 encoded by the nucleotide sequence of SEQ ID NO: 9. As for MCM8, sequence information has been registered with a plurality of accession numbers as mentioned above, and a plurality of transcript variants are present. The nucleotide sequence of SEQ ID NO: 9 represents the nucleotide sequence of one of these transcript variants.

CCNB3 refers to cyclin-B3 encoded by CCNB3 gene. CCNB3 is present in various animals including humans, and its sequence information is also publicly known (e.g., human: NM_033670.2, NM_033031.2, XM_006724610.1 (NP_391990.1, NP_149020.2, XP_006724673.1), etc.; the numbers represent accession numbers of the NCBI database, and the nucleotide sequence and the amino acid sequence are indicated outside and inside the parentheses, respectively). As one example, the nucleotide sequence of the human CCNB3 gene registered in the database as NM_033670.2 is shown in SEQ ID NO: 11, and the amino acid sequence of the human CCNB3 protein encoded by this human CCNB3 gene is shown in SEQ ID NO: 12. In the present specification, CCNB3 is not limited to a protein consisting of the amino acid sequence of SEQ ID NO: 12 encoded by the nucleotide sequence of SEQ ID NO: 11. As for CCNB3, sequence information has been registered with a plurality of accession numbers as mentioned above, and a plurality of transcript variants are present. The nucleotide sequence of SEQ ID NO: 11 represents the nucleotide sequence of one of these transcript variants.

MCMDC1 refers to mini-chromosome maintenance deficient domain containing 1 encoded by MCMDC1 gene. MCMDC1 is present in various animals including humans, and its sequence information is also publicly known (e.g., human: NM_017696.2, NM_153255.4 (NP_060166.2, NP_694987.1), etc.; the numbers represent accession numbers of the NCBI database, and the nucleotide sequence and the amino acid sequence are indicated outside and inside the parentheses, respectively). As one example, the nucleotide sequence of the human MCMDC1 gene registered in the database as NM_017696.2 is shown in SEQ ID NO: 13, and the amino acid sequence of the human MCMDC1 protein encoded by this human MCMDC1 gene is shown in SEQ ID NO: 14. In the present specification, MCMDC1 is not limited to a protein consisting of the amino acid sequence of SEQ ID NO: 14 encoded by the nucleotide sequence of SEQ ID NO: 13. As for MCMDC1, sequence information has been registered with a plurality of accession numbers as mentioned above, and a plurality of transcript variants are present. The nucleotide sequence of SEQ ID NO: 13 represents the nucleotide sequence of one of these transcript variants.

ATM is ATM serine/threonine kinase encoded by ATM gene and refers to a protein that belongs to the PI3/PI4 kinase family. ATM is present in various animals including humans, and its sequence information is also publicly known (e.g., human: NM_000051.3, XM_005271561.2, XM_005271562.2, XM_005271564.2, XM_006718843.1, XM_006718844.1, XM_006718845.1 (NP_000042.3, XP_005271618.2, XP_005271619.2, XP_005271621.2, XP_006718906.1, XP_006718907.1, XP_006718908.1), etc.; the numbers represent accession numbers of the NCBI database, and the nucleotide sequence and the amino acid sequence are indicated outside and inside the parentheses, respectively). As one example, the nucleotide sequence of the human ATM gene registered in the database as NM_000051.3 is shown in SEQ ID NO: 15, and the amino acid sequence of the human ATM protein encoded by this human ATM gene is shown in SEQ ID NO: 16. In the present specification, ATM is not limited to a protein consisting of the amino acid sequence of SEQ ID NO: 16 encoded by the nucleotide sequence of SEQ ID NO: 15. As for ATM, sequence information has been registered with a plurality of accession numbers as mentioned above, and a plurality of transcript variants are present. The nucleotide sequence of SEQ ID NO: 15 represents the nucleotide sequence of one of these transcript variants.

CDC25A is phosphatase that is encoded by CDC25A gene and belongs to the CDC25 family, and refers to a protein that activates CDC2 by dephosphorylation. CDC25A is present in various animals including humans, and its sequence information is also publicly known (e.g., human: NM_001789.2, NM_201567.1, XM_006713434.1, XM_006713435.1, XM_006713436.1 (NP_001780.2, NP_963861.1, XP_006713497.1, XP_006713498.1, XP_006713499.1), etc.; the numbers represent accession numbers of the NCBI database, and the nucleotide sequence and the amino acid sequence are indicated outside and inside the parentheses, respectively). As one example, the nucleotide sequence of the human CDC25A gene registered in the database as NM_001789.2 is shown in SEQ ID NO: 17, and the amino acid sequence of the human CDC25A protein encoded by this human CDC25A gene is shown in SEQ ID NO: 18. In the present specification, CDC25A is not limited to a protein consisting of the amino acid sequence of SEQ ID NO: 18 encoded by the nucleotide sequence of SEQ ID NO: 17. As for CDC25A, sequence information has been registered with a plurality of accession numbers as mentioned above, and a plurality of transcript variants are present. The nucleotide sequence of SEQ ID NO: 17 represents the nucleotide sequence of one of these transcript variants.

PRKDC is a catalytic subunit protein of DNA-dependent protein kinase encoded by PRKDC gene and refers to a protein that belongs to the PI3/PI4 kinase family. PRKDC is present in various animals including humans, and its sequence information is also publicly known (e.g., human: NM_006904.6, NM_001081640.1 (NP_008835.5, NP_001075109.1), etc.; the numbers represent accession numbers of the NCBI database, and the basic acid sequence and the amino acid sequence are indicated outside and inside the parentheses, respectively). As one example, the nucleotide sequence of the human PRKDC gene registered in the database as NM_006904.6 is shown in SEQ ID NO: 19, and the amino acid sequence of the human PRKDC protein encoded by this human PRKDC gene is shown in SEQ ID NO: 20. In the present specification, PRKDC is not limited to a protein consisting of the amino acid sequence of SEQ ID NO: 20 encoded by the nucleotide sequence of SEQ ID NO: 19. As for PRKDC, sequence information has been registered with a plurality of accession numbers as mentioned above, and a plurality of transcript variants are present. The nucleotide sequence of SEQ ID NO: 19 represents the nucleotide sequence of one of these transcript variants.

RBBP8 is retinoblastoma binding protein 8 encoded by RBBP8 gene and refers to a nuclear protein that binds directly to retinoblastoma protein. RBBP8 is present in various animals including humans, and its sequence information is also publicly known (e.g., human: NM_002894.2, NM_203291.1, NM_203292.1, XM_005258325.1, XM_005258326.1, XM_006722519.1, XM_006722520.1, XM_006722521.1, XM_006722522.1 (NP_002885.1, NP_976036.1, NP_976037.1, XP_005258382.1, XP_005258383.1, XP_006722582.1, XP_006722583.1, XP_006722584.1, XP_006722585.1), etc.; the numbers represent accession numbers of the NCBI database, and the nucleotide sequence and the amino acid sequence are indicated outside and inside the parentheses, respectively). As one example, the nucleotide sequence of the human RBBP8 gene registered in the database as NM_002894.2 is shown in SEQ ID NO: 21, and the amino acid sequence of the human RBBP8 protein encoded by this human RBBP8 gene is shown in SEQ ID NO: 22. In the present specification, RBBP8 is not limited to a protein consisting of the amino acid sequence of SEQ ID NO: 22 encoded by the nucleotide sequence of SEQ ID NO: 21. As for RBBP8, sequence information has been registered with a plurality of accession numbers as mentioned above, and a plurality of transcript variants are present. The nucleotide sequence of SEQ ID NO: 21 represents the nucleotide sequence of one of these transcript variants.

SKP2 is S-phase kinase-associated protein 2 encoded by SKP2 gene and refers to a protein that belongs to the Fbox protein, which is one of four subunits of E3 ubiquitin protein ligase. SKP2 is present in various animals including humans, and its sequence information is also publicly known (e.g., human: NM_005983.3, NM_032637.3, NM_001243120.1, XM_006714487.1 (NP_005974.2, NP_116026.1, NP_001230049.1, XP_006714550.1), etc.; the numbers represent accession numbers of the NCBI database, and the nucleotide sequence and the amino acid sequence are indicated outside and inside the parentheses, respectively). As one example, the nucleotide sequence of the coding region of the human SKP2 gene registered in the database as NM_005983.3 is shown in SEQ ID NO: 23, and the amino acid sequence of the human SKP2 protein encoded by this human SKP2 gene is shown in SEQ ID NO: 24. In the present specification, SKP2 is not limited to a protein consisting of the amino acid sequence of SEQ ID NO: 24 encoded by the nucleotide sequence of SEQ ID NO: 23. As for SKP2, sequence information has been registered with a plurality of accession numbers as mentioned above, and a plurality of transcript variants are present. The nucleotide sequence of SEQ ID NO: 23 represents the nucleotide sequence of one of these transcript variants.

MCM10 refers to mini-chromosome maintenance 10 encoded by MCM10 gene. MCM10 is present in various animals including humans, and its sequence information is also publicly known (e.g., human: NM_182751.2, NM_018518.4 (NP_877428.1, NP_060988.3), etc.; the numbers represent accession numbers of the NCBI database, and the nucleotide sequence and the amino acid sequence are indicated outside and inside the parentheses, respectively). As one example, the nucleotide sequence of the human MCM10 gene registered in the database as NM_182751.2 is shown in SEQ ID NO: 25, and the amino acid sequence of the human MCM10 protein encoded by this human MCM10 gene is shown in SEQ ID NO: 26. In the present specification, MCM10 is not limited to a protein consisting of the amino acid sequence of SEQ ID NO: 26 encoded by the nucleotide sequence of SEQ ID NO: 25. As for MCM10, sequence information has been registered with a plurality of accession numbers as mentioned above, and a plurality of transcript variants are present. The nucleotide sequence of SEQ ID NO: 25 represents the nucleotide sequence of one of these transcript variants.

CENPH is centromere protein H encoded by CENPH gene and refers to one of proteins constituting activated kinetochore located on the centromere. CENPH is present in various animals including humans, and its sequence information is also publicly known (e.g., human: NM_022909.3 (NP_075060.1), etc.; the numbers represent accession numbers of the NCBI database, and the nucleotide sequence and the amino acid sequence are indicated outside and inside the parentheses, respectively). As one example, the nucleotide sequence of the human CENPH gene registered in the database as NM_022909.3 is shown in SEQ ID NO: 27, and the amino acid sequence of the human CENPH protein encoded by this human CENPH gene is shown in SEQ ID NO: 28. In the present specification, CENPH is not limited to a protein consisting of the amino acid sequence of SEQ ID NO: 28 encoded by the nucleotide sequence of SEQ ID NO: 27. As for CENPH, there is the possibility that a plurality of transcript variants are present. The nucleotide sequence of SEQ ID NO: 27 represents the nucleotide sequence of a transcript variant.

BRSK1 is serine/threonine kinase encoded by BRSK1 gene and refers to kinase that acts at cell cycle checkpoint in DNA damage. BRSK1 is present in various animals including humans, and its sequence information is also publicly known (e.g., human: NM_032430.1, XM_005259327.1, XR_430213.1 (NP_115806.1, XP_005259384.1), etc.; the numbers represent accession numbers of the NCBI database, and the nucleotide sequence and the amino acid sequence are indicated outside and inside the parentheses, respectively). As one example, the nucleotide sequence of the human BRSK1 gene registered in the database as NM_032430.1 is shown in SEQ ID NO: 29, and the amino acid sequence of the human BRSK1 protein encoded by this human BRSK1 gene is shown in SEQ ID NO: 30. In the present specification, BRSK1 is not limited to a protein consisting of the amino acid sequence of SEQ ID NO: 30 encoded by the nucleotide sequence of SEQ ID NO: 29. As for BRSK1, sequence information has been registered with a plurality of accession numbers as mentioned above, and a plurality of transcript variants are present. The nucleotide sequence of SEQ ID NO: 29 represents the nucleotide sequence of one of these transcript variants.

MYLK, myosin light chain kinase, is a calcium/calmodulin-dependent enzyme that phosphorylates myosin regulatory light chains so that myosin interaction with actin filaments is facilitated to produce contractile activity. The gene encoding MYLK encodes both smooth muscle and non-muscle isoforms. MYLK is present in various animals including humans, and its sequence information is also publicly known (e.g., human: NM_053028.3, NM_053026.3, NM_053027.3, NM_053025.3, NM_053031.2, NM_053032.2, XM_011512862.1, XM_011512861.1, XM_011512860.1 (NP_444256.3, NP_444254.3, NP_444255.3, NP_444253.3, NP_444259.1, NP_444260.1, XP_011511164.1, XP_011511163.1, XP_011511162.1), etc.; the numbers represent accession numbers of the NCBI database, and the nucleotide sequence and the amino acid sequence are indicated outside and inside the parentheses, respectively). As one example, the nucleotide sequence of the human MYLK gene registered in the database as NM_053028.3 is shown in SEQ ID NO: 41, and the amino acid sequence of the human MYLK protein encoded by this human MYLK gene is shown in SEQ ID NO: 42. In the present specification, MYLK is not limited to a protein consisting of the amino acid sequence of SEQ ID NO: 42 encoded by the nucleotide sequence of SEQ ID NO: 41. As for MYLK, sequence information has been registered with a plurality of accession numbers as mentioned above, and a plurality of transcript variants are present. The nucleotide sequence of SEQ ID NO: 41 represents the nucleotide sequence of one of these transcript variants.

On the other hand, the protein having the function of suppressively participating in apoptosis means a protein having the function of inhibiting apoptosis by inhibiting mechanisms such as karyopyknosis, cell contraction, membrane blebbing, and DNA fragmentation. The function of suppressively participating in apoptosis is meant to include both of the function of inhibiting apoptosis and the function of inhibiting a factor promoting apoptosis. Examples of the factor promoting apoptosis can include many factors such as caspase, Fas, and TNFR.

Specifically, examples of the anti-apoptosis-related protein that exhibits synthetic lethality when inhibited together with GST-π can include at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A. Of these 20 types of anti-apoptosis-related proteins, one type of anti-apoptosis-related protein may be inhibited together with GST-π, or two or more types of anti-apoptosis-related proteins may be inhibited together with GST-π.

AATF was identified on the basis of its interaction with MAP3K12/DLK, a protein kinase known to be involved in the induction of cell apoptosis. AATF contains a leucine zipper, which is a characteristic motif of transcription factors, and has been shown to exhibit strong transactivation activity when fused to Gal4 DNA-binding domain. Overexpression of the gene encoding AATF is known to inhibit MAP3K12-induced apoptosis. AATF is present in various animals including humans, and its sequence information is also publicly known (e.g., human: NM_012138.3, XM_011546799.1, XM_011524611.1, XR_951958.1, XR_934439.1 (NP_036270.1, XP_011545101.1, XP_011522913.1), etc.; the numbers represent accession numbers of the NCBI database, and the nucleotide sequence and the amino acid sequence are indicated outside and inside the parentheses, respectively). As one example, the nucleotide sequence of the human AATF gene registered in the database as NM_012138.3 is shown in SEQ ID NO: 39, and the amino acid sequence of the human AATF protein encoded by this human AATF gene is shown in SEQ ID NO: 40. In the present specification, AATF is not limited to a protein consisting of the amino acid sequence of SEQ ID NO: 40 encoded by the nucleotide sequence of SEQ ID NO: 39. As for AATF, sequence information has been registered with a plurality of accession numbers as mentioned above, and a plurality of transcript variants are present. The nucleotide sequence of SEQ ID NO: 39 represents the nucleotide sequence of one of these transcript variants.

ALOX12, arachidonate 12-lipoxygenase, is known to be involved in atherosclerosis, osteoporosis, and the like. ALOX12 is also known to positively regulate angiogenesis through regulation of the expression of the vascular endothelial growth factor and to play a role in apoptotic process by promoting the survival of vascular smooth muscle cells or the like. ALOX12 is present in various animals including humans, and its sequence information is also publicly known (e.g., human: NM_000697.2, XM_011523780.1 (NP_000688.2, XP_011522082.1, AAH69557.1), etc.; the numbers represent accession numbers of the NCBI database, and the nucleotide sequence and the amino acid sequence are indicated outside and inside the parentheses, respectively). As one example, the nucleotide sequence of the human ALOX12 gene registered in the database as NM_000697.2 is shown in SEQ ID NO: 43, and the amino acid sequence of the human ALOX12 protein encoded by this human ALOX12 gene is shown in SEQ ID NO: 44. In the present specification, ALOX12 is not limited to a protein consisting of the amino acid sequence of SEQ ID NO: 44 encoded by the nucleotide sequence of SEQ ID NO: 43. As for ALOX12, sequence information has been registered with a plurality of accession numbers as mentioned above, and a plurality of transcript variants are present. The nucleotide sequence of SEQ ID NO: 43 represents the nucleotide sequence of one of these transcript variants.

ANXA1 is a membrane-localized protein that binds to phospholipids. ANXA1 inhibits phospholipase A2 and has anti-inflammatory activity. ANXA1 is present in various animals including humans, and its sequence information is also publicly known (e.g., human: NM_000700.2, XM_011518609.1, XM_011518608.1 (NP_000691.1, AAH34157.1), etc.; the numbers represent accession numbers of the NCBI database, and the nucleotide sequence and the amino acid sequence are indicated outside and inside the parentheses, respectively). As one example, the nucleotide sequence of the human ANXA1 gene registered in the database as NM_000700.2 is shown in SEQ ID NO: 45, and the amino acid sequence of the human ANXA1 protein encoded by this human ANXA1 gene is shown in SEQ ID NO: 46. In the present specification, ANXA1 is not limited to a protein consisting of the amino acid sequence of SEQ ID NO: 46 encoded by the nucleotide sequence of SEQ ID NO: 45. As for ANXA1, sequence information has been registered with a plurality of accession numbers as mentioned above, and a plurality of transcript variants are present. The nucleotide sequence of SEQ ID NO: 45 represents the nucleotide sequence of one of these transcript variants.

ANXA4 belongs to the annexin family of calcium-dependent phospholipid-binding proteins. This protein has possible interactions with ATP and is known to have in vitro anticoagulant activity and to inhibit phospholipase A2. ANXA4 is present in various animals including humans, and its sequence information is also publicly known (e.g., human: NM_001153.3, XM_011532805.1 (NP_001144.1, XP_011531107.1, AAH63672.1, AAH00182.1, AAH11659.1), etc.; the numbers represent accession numbers of the NCBI database, and the nucleotide sequence and the amino acid sequence are indicated outside and inside the parentheses, respectively). As one example, the nucleotide sequence of the human ANXA4 gene registered in the database as NM_001153.3 is shown in SEQ ID NO: 47, and the amino acid sequence of the human ANXA4 protein encoded by this human ANXA4 gene is shown in SEQ ID NO: 48. In the present specification, ANXA4 is not limited to a protein consisting of the amino acid sequence of SEQ ID NO: 48 encoded by the nucleotide sequence of SEQ ID NO: 47. As for ANXA4, sequence information has been registered with a plurality of accession numbers as mentioned above, and a plurality of transcript variants are present. The nucleotide sequence of SEQ ID NO: 47 represents the nucleotide sequence of one of these transcript variants.

API5 is an apoptosis inhibitory protein whose expression is known to prevent apoptosis after growth factor deprivation. API5 suppresses the transcription factor E2F1-induced apoptosis and also interacts with and negatively regulates Acinus, a nuclear factor involved in apoptotic DNA fragmentation. API5 is present in various animals including humans, and its sequence information is also publicly known (e.g., human: NM_001142930.1, NM_006595.3, NM_001243747.1, NM_001142931.1, XM_006718359.2, NR_024625.1 (NP_001136402.1, NP_001136403.1, NP_001230676.1, NP_006586.1, XP_006718422.1), etc.; the numbers represent accession numbers of the NCBI database, and the nucleotide sequence and the amino acid sequence are indicated outside and inside the parentheses, respectively). As one example, the nucleotide sequence of the human API5 gene registered in the database as NM_001142930.1 is shown in SEQ ID NO: 49, and the amino acid sequence of the human API5 protein encoded by this human API5 gene is shown in SEQ ID NO: 50. In the present specification, API5 is not limited to a protein consisting of the amino acid sequence of SEQ ID NO: 50 encoded by the nucleotide sequence of SEQ ID NO: 49. As for API5, sequence information has been registered with a plurality of accession numbers as mentioned above, and a plurality of transcript variants are present. The nucleotide sequence of SEQ ID NO: 49 represents the nucleotide sequence of one of these transcript variants.

ATF5 is known to be involved in diseases caused by human T-cell leukemia virus type 1. ATF5 is a transcriptional activator that binds to the cAMP response element (CRE) present in many viral promoters, etc., and is known to inhibit the differentiation of neuroprogenitor cells into neurons. ATF5 is present in various animals including humans, and its sequence information is also publicly known (e.g., human: NM_012068.5, NM_001193646.1, NM_001290746.1, XM_011526629.1 (NP_036200.2, NP_001277675.1, NP_001180575.1, XP_011524931.1), etc.; the numbers represent accession numbers of the NCBI database, and the nucleotide sequence and the amino acid sequence are indicated outside and inside the parentheses, respectively). As one example, the nucleotide sequence of the human ATF5 gene registered in the database as NM_012068.5 is shown in SEQ ID NO: 51, and the amino acid sequence of the human ATF5 protein encoded by this human ATF5 gene is shown in SEQ ID NO: 52. In the present specification, ATF5 is not limited to a protein consisting of the amino acid sequence of SEQ ID NO: 52 encoded by the nucleotide sequence of SEQ ID NO: 51. As for ATF5, sequence information has been registered with a plurality of accession numbers as mentioned above, and a plurality of transcript variants are present. The nucleotide sequence of SEQ ID NO: 51 represents the nucleotide sequence of one of these transcript variants.

AVEN is a protein known as an apoptosis, caspase activation inhibitor and is known to be involved in schizoid personality disorder and alexithymia. AVEN is also known to inhibit apoptosis mediated by Apaf-1. AVEN is present in various animals including humans, and its sequence information is also publicly known (e.g., human: NM_020371.2, XM_011521820.1, XM_005254563.2, XM_011521819.1, XM_011521818.1 (NP NP_065104.1, XP_011520122.1, XP_011520121.1, XP_011520120.1, XP_005254620.1, AAH63533.1, AAF91470.1), etc.; the numbers represent accession numbers of the NCBI database, and the nucleotide sequence and the amino acid sequence are indicated outside and inside the parentheses, respectively). As one example, the nucleotide sequence of the human AVEN gene registered in the database as NM_020371.2 is shown in SEQ ID NO: 53, and the amino acid sequence of the human AVEN protein encoded by this human AVEN gene is shown in SEQ ID NO: 54. In the present specification, AVEN is not limited to a protein consisting of the amino acid sequence of SEQ ID NO: 54 encoded by the nucleotide sequence of SEQ ID NO: 53. As for AVEN, sequence information has been registered with a plurality of accession numbers as mentioned above, and a plurality of transcript variants are present. The nucleotide sequence of SEQ ID NO: 53 represents the nucleotide sequence of one of these transcript variants.

AZU1 is a protein contained in azurophil granules and has monocyte chemotactic and antimicrobial activity. AZU1 is an important multifunctional inflammatory mediator. AZU1 is present in various animals including humans, and its sequence information is also publicly known (e.g., human: NM_001700.3 (NP_001691.1, EAW69592.1, AAH93933.1, AAH93931.1, AAH69495.1), etc.; the numbers represent accession numbers of the NCBI database, and the nucleotide sequence and the amino acid sequence are indicated outside and inside the parentheses, respectively). As one example, the nucleotide sequence of the human AZU1 gene registered in the database as NM_001700.3 is shown in SEQ ID NO: 55, and the amino acid sequence of the human AZU1 protein encoded by this human AZU1 gene is shown in SEQ ID NO: 56. In the present specification, AZU1 is not limited to a protein consisting of the amino acid sequence of SEQ ID NO: 56 encoded by the nucleotide sequence of SEQ ID NO: 55. As for AZU1, sequence information has been registered with a plurality of accession numbers as mentioned above, and a plurality of transcript variants are present. The nucleotide sequence of SEQ ID NO: 55 represents the nucleotide sequence of one of these transcript variants.

BAG1 binds to BCL2, a membrane protein that inhibits a pathway leading to apoptosis or programmed cell death. BAG1 enhances the anti-apoptotic effects of BCL2 and represents a link between growth factor receptors and anti-apoptotic mechanisms. BAG1 is present in various animals including humans, and its sequence information is also publicly known (e.g., human: NM_004323.5, NM_001172415.1 (NP_004314.5, NP_001165886.1, AAH14774.2), etc.; the numbers represent accession numbers of the NCBI database, and the nucleotide sequence and the amino acid sequence are indicated outside and inside the parentheses, respectively). As one example, the nucleotide sequence of the human BAG1 gene registered in the database as NM_004323.5 is shown in SEQ ID NO: 57, and the amino acid sequence of the human BAG1 protein encoded by this human BAG1 gene is shown in SEQ ID NO: 58. In the present specification, BAG1 is not limited to a protein consisting of the amino acid sequence of SEQ ID NO: 58 encoded by the nucleotide sequence of SEQ ID NO: 57. As for BAG1, sequence information has been registered with a plurality of accession numbers as mentioned above, and a plurality of transcript variants are present. The nucleotide sequence of SEQ ID NO: 57 represents the nucleotide sequence of one of these transcript variants.

BCL2L1 belongs to the BCL-2 protein family. Members of this protein family form hetero- or homodimers and act as anti- or pro-apoptotic regulators that are involved in a wide variety of cellular activities. BCL2L1 is located at the outer mitochondrial membrane and has been shown to regulate outer mitochondrial membrane channel opening. BCL2L1 is present in various animals including humans, and its sequence information is also publicly known (e.g., human: NM_138578.1, NM_001191.2, XM_011528966.1, XM_011528965.1, XM_011528961.1, XM_011528960.1, XM_011528964.1, XM_011528963.1, XM_011528962.1, XM_005260487.3, XM_005260486.2 (NP_612815.1, NP_001182.1, AAH19307.1, XP_011527268.1, XP_011527267.1, XP_011527266.1, XP_011527265.1, XP_011527264.1, XP_011527263.1, XP_011527262.1, XP_005260544.1, XP_005260543.1), etc.; the numbers represent accession numbers of the NCBI database, and the nucleotide sequence and the amino acid sequence are indicated outside and inside the parentheses, respectively). As one example, the nucleotide sequence of the human BCL2L1 gene registered in the database as NM_138578.1 is shown in SEQ ID NO: 59, and the amino acid sequence of the human BCL2L1 protein encoded by this human BCL2L1 gene is shown in SEQ ID NO: 60. In the present specification, BCL2L1 is not limited to a protein consisting of the amino acid sequence of SEQ ID NO: 60 encoded by the nucleotide sequence of SEQ ID NO: 59. As for BCL2L1, sequence information has been registered with a plurality of accession numbers as mentioned above, and a plurality of transcript variants are present. The nucleotide sequence of SEQ ID NO: 59 represents the nucleotide sequence of one of these transcript variants.

BFAR, a bifunctional apoptosis regulator, has anti-apoptotic activity, both against apoptosis triggered via cell death-receptors and against apoptosis triggered via mitochondrial factors. BFAR is present in various animals including humans, and its sequence information is also publicly known (e.g., human: NM_016561.2, XM_006725196.2, XM_011546704.1, XM_005255350.2, XM_011522520.1 (NP_057645.1, XP_011545006.1, XP_011520822.1, XP_006725259.1, XP_005255407.1, AAH03054.1), etc.; the numbers represent accession numbers of the NCBI database, and the nucleotide sequence and the amino acid sequence are indicated outside and inside the parentheses, respectively). As one example, the nucleotide sequence of the human BFAR gene registered in the database as NM_016561.2 is shown in SEQ ID NO: 61, and the amino acid sequence of the human BFAR protein encoded by this human BFAR gene is shown in SEQ ID NO: 62. In the present specification, BFAR is not limited to a protein consisting of the amino acid sequence of SEQ ID NO: 62 encoded by the nucleotide sequence of SEQ ID NO: 61. As for BFAR, sequence information has been registered with a plurality of accession numbers as mentioned above, and a plurality of transcript variants are present. The nucleotide sequence of SEQ ID NO: 61 represents the nucleotide sequence of one of these transcript variants.

CFLAR, a regulator of apoptosis, is known to be structurally similar to caspase-8. However, CFLAR lacks caspase activity and is cleaved into two peptides by caspase-8. CFLAR is present in various animals including humans, and its sequence information is also publicly known (e.g., human: NM_003879.5, NM_001202519.1, NM_001202518.1, NM_001308043.1, NM_001308042.1, NM_001202517.1, NM_001202516.1, NM_001127184.2, NM_001202515.1, NM_001127183.2, XM_011512100.1 (NP_003870.4, NP_001294972.1, NP_001294971.1, NP_001189448.1, NP_001189446.1, NP_001189445.1, NP_001189444.1, NP_001120656.1, XP_011510402.1), etc.; the numbers represent accession numbers of the NCBI database, and the nucleotide sequence and the amino acid sequence are indicated outside and inside the parentheses, respectively). As one example, the nucleotide sequence of the human CFLAR gene registered in the database as NM_003879.5 is shown in SEQ ID NO: 63, and the amino acid sequence of the human CFLAR protein encoded by this human CFLAR gene is shown in SEQ ID NO: 64. In the present specification, CFLAR is not limited to a protein consisting of the amino acid sequence of SEQ ID NO: 64 encoded by the nucleotide sequence of SEQ ID NO: 63. As for CFLAR, sequence information has been registered with a plurality of accession numbers as mentioned above, and a plurality of transcript variants are present. The nucleotide sequence of SEQ ID NO: 63 represents the nucleotide sequence of one of these transcript variants.

IL2, interleukin 2, is a secreted cytokine that is important for the proliferation of T and B lymphocytes. IL2 is present in various animals including humans, and its sequence information is also publicly known (e.g., human: NM_000586.3 (NP_000577.2), etc.; the numbers represent accession numbers of the NCBI database, and the nucleotide sequence and the amino acid sequence are indicated outside and inside the parentheses, respectively). As one example, the nucleotide sequence of the human IL2 gene registered in the database as NM_000586.3 is shown in SEQ ID NO: 65, and the amino acid sequence of the human IL2 protein encoded by this human IL2 gene is shown in SEQ ID NO: 66. In the present specification, IL2 is not limited to a protein consisting of the amino acid sequence of SEQ ID NO: 66 encoded by the nucleotide sequence of SEQ ID NO: 65. As for IL2, there is the possibility that a plurality of transcript variants are present. The nucleotide sequence of SEQ ID NO: 65 represents the nucleotide sequence of a transcript variant.

MALT1 is encoded by a gene that is recurrently rearranged in chromosomal translocation with baculoviral IAP repeat-containing protein 3 (also known as apoptosis inhibitor 2) and immunoglobulin heavy chain locus in mucosa-associated lymphoid tissue lymphomas. MALT1 may activate NFκB. MALT1 is present in various animals including humans, and its sequence information is also publicly known (e.g., human: NM_173844.2, NM_006785.3, XM_011525794.1 (NP_776216.1, NP_006776.1, XP_011524096.1), etc.; the numbers represent accession numbers of the NCBI database, and the nucleotide sequence and the amino acid sequence are indicated outside and inside the parentheses, respectively). As one example, the nucleotide sequence of the human MALT1 gene registered in the database as NM_006785.3 is shown in SEQ ID NO: 67, and the amino acid sequence of the human MALT1 protein encoded by this human MALT1 gene is shown in SEQ ID NO: 68. In the present specification, MALT1 is not limited to a protein consisting of the amino acid sequence of SEQ ID NO: 68 encoded by the nucleotide sequence of SEQ ID NO: 67. As for MALT1, sequence information has been registered with a plurality of accession numbers as mentioned above, and a plurality of transcript variants are present. The nucleotide sequence of SEQ ID NO: 67 represents the nucleotide sequence of one of these transcript variants.

MCL1 is an anti-apoptotic protein, which is a member of the Bcl-2 family. The longest variant resulting from the alternative splicing of the MCL1 gene enhances cell survival by inhibiting apoptosis, while the alternatively spliced shorter variants promote apoptosis and induce cell death. MCL1 is present in various animals including humans, and its sequence information is also publicly known (e.g., human: NM_021960.4, NM_001197320.1, NM_182763.2 (NP_068779.1, NP_001184249.1, NP_877495.1), etc.; the numbers represent accession numbers of the NCBI database, and the nucleotide sequence and the amino acid sequence are indicated outside and inside the parentheses, respectively). As one example, the nucleotide sequence of the human MCL1 gene registered in the database as NM_021960.4 is shown in SEQ ID NO: 69, and the amino acid sequence of the human MCL1 protein encoded by this human MCL1 gene is shown in SEQ ID NO: 70. In the present specification, MCL1 is not limited to a protein consisting of the amino acid sequence of SEQ ID NO: 70 encoded by the nucleotide sequence of SEQ ID NO: 69. As for MCL1, sequence information has been registered with a plurality of accession numbers as mentioned above, and a plurality of transcript variants are present. The nucleotide sequence of SEQ ID NO: 69 represents the nucleotide sequence of one of these transcript variants.

MKL1 is known to interact with the transcription factor myocardin, a key regulator of smooth muscle cell differentiation. MKL1 is predominantly nuclear and helps transduce signals from the cytoskeleton to the nucleus. The MKL1 gene is involved in a translocation event that creates a fusion of this gene and the RNA-binding motif protein-15 gene. MKL1 is present in various animals including humans, and its sequence information is also publicly known (e.g., human: NM_001282662.1, NM_001282660.1, NM_020831.4, NM_001282661.1, XM_011530287.1, XM_011530286.1, XM_011530285.1, XM_011530284.1, XM_011530283.1, XM_005261691.3 (NP_001269591.1, NP_001269589.1, NP_065882.1, NP_001269590.1, XP_011528589.1, XP_011528588.1, XP_011528587.1, XP_011528586.1, XP_011528585.1, XP_005261751.1, XP_005261749.1, XP_005261748.1), etc.; the numbers represent accession numbers of the NCBI database, and the nucleotide sequence and the amino acid sequence are indicated outside and inside the parentheses, respectively). As one example, the nucleotide sequence of the human MKL1 gene registered in the database as NM_001282662.1 is shown in SEQ ID NO: 71, and the amino acid sequence of the human MKL1 protein encoded by this human MKL1 gene is shown in SEQ ID NO: 72. In the present specification, MKL1 is not limited to a protein consisting of the amino acid sequence of SEQ ID NO: 72 encoded by the nucleotide sequence of SEQ ID NO: 71. As for MKL1, sequence information has been registered with a plurality of accession numbers as mentioned above, and a plurality of transcript variants are present. The nucleotide sequence of SEQ ID NO: 71 represents the nucleotide sequence of one of these transcript variants.

MPO, myeloperoxidase, is a heme protein that is synthesized during myeloid differentiation and constitutes the major component of neutrophil azurophil granules. MPO produces hypohalous acids central to the microbicidal activity of neutrophils. MPO is present in various animals including humans, and its sequence information is also publicly known (e.g., human: NM_000250.1, XM_011524823.1, XM_011524822.1, XM_011524821.1 (NP_000241.1), etc.; the numbers represent accession numbers of the NCBI database, and the nucleotide sequence and the amino acid sequence are indicated outside and inside the parentheses, respectively). As one example, the nucleotide sequence of the human MPO gene registered in the database as NM_000250.1 is shown in SEQ ID NO: 73, and the amino acid sequence of the human MPO protein encoded by this human MPO gene is shown in SEQ ID NO: 74. In the present specification, MPO is not limited to a protein consisting of the amino acid sequence of SEQ ID NO: 74 encoded by the nucleotide sequence of SEQ ID NO: 73. As for MPO, sequence information has been registered with a plurality of accession numbers as mentioned above, and a plurality of transcript variants are present. The nucleotide sequence of SEQ ID NO: 73 represents the nucleotide sequence of one of these transcript variants.

MTL5, a metallothionein-like protein, has been shown to be expressed specifically in the mouse testis and ovary. Metallothionein may play a central role in the regulation of cell growth and differentiation and be involved in spermatogenesis. MTL5 is present in various animals including humans, and its sequence information is also publicly known (e.g., human: NM_004923.3, NM_001039656.1, XM_011545404.1, XM_011545403.1, XM_011545402.1 (NP_001034745.1, NP_004914.2, XP_011543706.1, XP_011543705.1, XP_011543704.1), etc.; the numbers represent accession numbers of the NCBI database, and the nucleotide sequence and the amino acid sequence are indicated outside and inside the parentheses, respectively). As one example, the nucleotide sequence of the human MTL5 gene registered in the database as NM_004923.3 is shown in SEQ ID NO: 75, and the amino acid sequence of the human MTL5 protein encoded by this human MTL5 gene is shown in SEQ ID NO: 76. In the present specification, MTL5 is not limited to a protein consisting of the amino acid sequence of SEQ ID NO: 76 encoded by the nucleotide sequence of SEQ ID NO: 75. As for MTL5, sequence information has been registered with a plurality of accession numbers as mentioned above, and a plurality of transcript variants are present. The nucleotide sequence of SEQ ID NO: 75 represents the nucleotide sequence of one of these transcript variants.

MYBL2 is a nuclear protein that belongs to the MYB family of transcription factors and is involved in cell cycle progression. MYBL2 is phosphorylated by cyclin A/cyclin-dependent kinase 2 during the S-phase of the cell cycle and possesses both activator and repressor activities. MYBL2 is present in various animals including humans, and its sequence information is also publicly known (e.g., human: NM_001278610.1, NM_002466.3 (NP_001265539.1, NP_002457.1), etc.; the numbers represent accession numbers of the NCBI database, and the nucleotide sequence and the amino acid sequence are indicated outside and inside the parentheses, respectively). As one example, the nucleotide sequence of the human MYBL2 gene registered in the database as NM_002466.3 is shown in SEQ ID NO: 77, and the amino acid sequence of the human MYBL2 protein encoded by this human MYBL2 gene is shown in SEQ ID NO: 78. In the present specification, MYBL2 is not limited to a protein consisting of the amino acid sequence of SEQ ID NO: 78 encoded by the nucleotide sequence of SEQ ID NO: 77. As for MYBL2, sequence information has been registered with a plurality of accession numbers as mentioned above, and a plurality of transcript variants are present. The nucleotide sequence of SEQ ID NO: 77 represents the nucleotide sequence of one of these transcript variants.

MYO18A, myosin 18A, is known to be involved in 8p11 myeloproliferative syndrome. MYO18A has motor activity and ATPase activity. MYO18A is present in various animals including humans, and its sequence information is also publicly known (e.g., human: NM_203318.1, NM_078471.3 (NP_976063.1, NP_510880.2), etc.; the numbers represent accession numbers of the NCBI database, and the nucleotide sequence and the amino acid sequence are indicated outside and inside the parentheses, respectively). As one example, the nucleotide sequence of the human MYO18A gene registered in the database as NM_078471.3 is shown in SEQ ID NO: 79, and the amino acid sequence of the human MYO18A protein encoded by this human MYO18A gene is shown in SEQ ID NO: 80. In the present specification, MYO18A is not limited to a protein consisting of the amino acid sequence of SEQ ID NO: 80 encoded by the nucleotide sequence of SEQ ID NO: 79. As for MYO18A, sequence information has been registered with a plurality of accession numbers as mentioned above, and a plurality of transcript variants are present. The nucleotide sequence of SEQ ID NO: 79 represents the nucleotide sequence of one of these transcript variants.

On the other hand, the PI3K signaling pathway-related protein is a protein, except for Akt1, included in proteins involved in the PI3K/AKT signaling pathway. In other words, the PI3K signaling pathway-related protein is a PI3K/AKT signaling pathway-related protein except for Akt1. The PI3K/AKT signaling pathway is disclosed in, for example, Cell 2007 Jun. 29; 129 (7): 1261-74.

Specifically, examples of the PI3K signaling pathway-related protein that exhibits synthetic lethality when inhibited together with GST-π can include at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF. Of these 14 types of PI3K signaling pathway-related proteins, one type of PI3K signaling pathway-related protein may be inhibited together with GST-π, or two or more types of PI3K signaling pathway-related proteins may be inhibited together with GST-π.

Particularly, it is preferable for the PI3K signaling pathway-related protein that at least one PI3K signaling pathway-related protein selected from the group consisting of MTOR, IRAK1, IRS1, MYD88, NFKB1, PIK3CG, and RAC1 should be inhibited together with GST-π. These 7 types of PI3K signaling pathway-related proteins exhibit a remarkably high cell growth-inhibiting effect when each inhibited together with GST-π compared with when inhibited each alone or when GST-π is inhibited alone. Thus, it is preferable that the PI3K signaling pathway-related protein that exhibits synthetic lethality when inhibited together with GST-π should be selected from these 7 types of PI3K signaling pathway-related proteins.

MTOR, mechanistic target of rapamycin, is a target protein of rapamycin and is known as a serine-threonine kinase. MTOR belongs to the phosphatidylinositol kinase-related kinase family and is known to mediate the response of cells to stresses such as DNA damage and nutrient deprivation. MTOR is also known to act as a target of cell cycle arrest and the immunosuppressive effect of FKBP12-rapamycin complexes. MTOR is present in various animals including humans, and its sequence information is also publicly known (e.g., human: NM_004958.3, XM_005263438.1, XM_011541166.1 (NP_004949.1, XP_005263495.1, XP_005263495.1), etc.; the numbers represent accession numbers of the NCBI database, and the nucleotide sequence and the amino acid sequence are indicated outside and inside the parentheses, respectively). As one example, the nucleotide sequence of the human MTOR gene registered in the database as NM_004958.3 is shown in SEQ ID NO: 81, and the amino acid sequence of the human MTOR protein encoded by this human MTOR gene is shown in SEQ ID NO: 82. In the present specification, MTOR is not limited to a protein consisting of the amino acid sequence of SEQ ID NO: 82 encoded by the nucleotide sequence of SEQ ID NO: 81. As for MTOR, sequence information has been registered with a plurality of accession numbers as mentioned above, and a plurality of transcript variants are present. The nucleotide sequence of SEQ ID NO: 81 represents the nucleotide sequence of one of these transcript variants.

IRAK1, interleukin 1 receptor associated kinase 1, is a serine-threonine kinase that acts on interleukin 1 receptors by stimulation. IRAK1 is partly responsible for IL1-induced upregulation associated with a transcription factor NFκB. IRAK1 is present in various animals including humans, and its sequence information is also publicly known (e.g., human: NM_001025243.1, NM_001025242.1, NM_001569.3, XM_011531158.1, XM_005274668.2 (NP_001020414.1, NP_001020413.1, NP_001560.2, XP_011529460.1, XP_005274725.1), etc.; the numbers represent accession numbers of the NCBI database, and the nucleotide sequence and the amino acid sequence are indicated outside and inside the parentheses, respectively). As one example, the nucleotide sequence of the human IRAK1 gene registered in the database as NM_001025243.1 is shown in SEQ ID NO: 83, and the amino acid sequence of the human IRAK1 protein encoded by this human IRAK1 gene is shown in SEQ ID NO: 84. In the present specification, IRAK1 is not limited to a protein consisting of the amino acid sequence of SEQ ID NO: 84 encoded by the nucleotide sequence of SEQ ID NO: 83. As for IRAK1, sequence information has been registered with a plurality of accession numbers as mentioned above, and a plurality of transcript variants are present. The nucleotide sequence of SEQ ID NO: 83 represents the nucleotide sequence of one of these transcript variants.

IRS1, insulin receptor substrate 1, is known as a protein that is phosphorylated by insulin receptor tyrosine kinase. IRS1 is present in various animals including humans, and its sequence information is also publicly known (e.g., human: NM_005544.2 (NP_005535.1), etc.; the numbers represent accession numbers of the NCBI database, and the nucleotide sequence and the amino acid sequence are indicated outside and inside the parentheses, respectively). As one example, the nucleotide sequence of the human IRS1 gene registered in the database as NM_005544.2 is shown in SEQ ID NO: 85, and the amino acid sequence of the human IRS1 protein encoded by this human IRS1 gene is shown in SEQ ID NO: 86. In the present specification, IRS1 is not limited to a protein consisting of the amino acid sequence of SEQ ID NO: 86 encoded by the nucleotide sequence of SEQ ID NO: 85. As for IRS1, there is the possibility that a plurality of transcript variants are present. The nucleotide sequence of SEQ ID NO: 85 represents the nucleotide sequence of one of these transcript variants.

MYD88, myeloid differentiation primary response 88, is a cytoplasmic adaptor protein that plays a key role in innate and adaptive immune responses. MYD88 functions as an essential signal transducer in the interleukin 1 and Toll-like receptor signaling pathways. These signaling pathways regulate the activation of many inflammatory genes. MYD88 has an N-terminal death domain and a C-terminal Toll-interleukin 1 receptor domain. MYD88 is present in various animals including humans, and its sequence information is also publicly known (e.g., human: NM_001172567.1, NM_002468.4, NM_001172569.1, NM_001172568.1, NM_001172566.1, XM_005265172.1, XM_006713170.1 (NP_001166038.1, NP_002459.2, NP_001166040.1, NP_001166039.1, NP_001166037.1, XP_005265229.1, XP_006713233.1), etc.; the numbers represent accession numbers of the NCBI database, and the nucleotide sequence and the amino acid sequence are indicated outside and inside the parentheses, respectively). As one example, the nucleotide sequence of the human MYD88 gene registered in the database as NM_001172567.1 is shown in SEQ ID NO: 87, and the amino acid sequence of the human MYD88 protein encoded by this human MYD88 gene is shown in SEQ ID NO: 88. In the present specification, MYD88 is not limited to a protein consisting of the amino acid sequence of SEQ ID NO: 88 encoded by the nucleotide sequence of SEQ ID NO: 87. As for MYD88, sequence information has been registered with a plurality of accession numbers as mentioned above, and a plurality of transcript variants are present. The nucleotide sequence of SEQ ID NO: 87 represents the nucleotide sequence of one of these transcript variants.

NFKB1, nuclear factor of kappa light polypeptide gene enhancer in B-cells 1, is a 105 kD protein that is processed into a 50 kD protein during translation by 26S proteasome. The 105 kD protein is a Rel protein-specific transcription inhibitor, and the 50 kD protein is a DNA-binding subunit in a NFκ-B protein (NFKB) complex. NFKB1 is present in various animals including humans, and its sequence information is also publicly known (e.g., human: NM_003998.3, NM_001165412.1, XM_011532006.1, XM_011532007.1, XM_011532008.1, XM_011532009.1 (NP_003989.2, NP_001158884.1, XP_011530308.1, XP_011530309.1, XP_011530310.1, XP_011530311.1), etc.; the numbers represent accession numbers of the NCBI database, and the nucleotide sequence and the amino acid sequence are indicated outside and inside the parentheses, respectively). As one example, the nucleotide sequence of the human NFKB1 gene registered in the database as NM_003998.3 is shown in SEQ ID NO: 89, and the amino acid sequence of the human NFKB1 protein encoded by this human NFKB1 gene is shown in SEQ ID NO: 90. In the present specification, NFKB1 is not limited to a protein consisting of the amino acid sequence of SEQ ID NO: 90 encoded by the nucleotide sequence of SEQ ID NO: 89. As for NFKB1, sequence information has been registered with a plurality of accession numbers as mentioned above, and a plurality of transcript variants are present. The nucleotide sequence of SEQ ID NO: 89 represents the nucleotide sequence of one of these transcript variants.

PIK3CG, phosphatidylinositol-4,5-bisphosphate 3-kinase, catalytic subunit gamma, is known to form PI3K by binding to a p85 regulatory subunit, as with other class 1 catalytic subunits (p110-α, p110-β, and p110-δ). PIK3CG is present in various animals including humans, and its sequence information is also publicly known (e.g., human: NM_001282426.1, NM_001282427.1, NM_002649.3, XM_011516316.1, XM_011516317.1, XM_005250443.2 (NP_001269355.1, NP_001269356.1, NP_002640.2, XP_011514618.1, XP_011514619.1, XP_005250500.1), etc.; the numbers represent accession numbers of the NCBI database, and the nucleotide sequence and the amino acid sequence are indicated outside and inside the parentheses, respectively). As one example, the nucleotide sequence of the human PIK3CG gene registered in the database as NM_001282426.1 is shown in SEQ ID NO: 91, and the amino acid sequence of the human PIK3CG protein encoded by this human PIK3CG gene is shown in SEQ ID NO: 92. In the present specification, PIK3CG is not limited to a protein consisting of the amino acid sequence of SEQ ID NO: 92 encoded by the nucleotide sequence of SEQ ID NO: 91. As for PIK3CG, sequence information has been registered with a plurality of accession numbers as mentioned above, and a plurality of transcript variants are present. The nucleotide sequence of SEQ ID NO: 91 represents the nucleotide sequence of one of these transcript variants.

RAC1, ras-related C3 botulinum toxin substrate 1 (rho family, small GTP binding protein Rac1), is known as GTPase belonging to the RAS superfamily as a small GTP-binding protein. Factors belonging to this superfamily regulate various intracellular events including growth, cytoskeletal reorganization, and protein kinase activation. RAC1 is present in various animals including humans, and its sequence information is also publicly known (e.g., human: NM_018890.3, NM_006908.4 (NP_061485.1, NP_008839.2), etc.; the numbers represent accession numbers of the NCBI database, and the nucleotide sequence and the amino acid sequence are indicated outside and inside the parentheses, respectively). As one example, the nucleotide sequence of the human RAC1 gene registered in the database as NM_018890.3 is shown in SEQ ID NO: 93, and the amino acid sequence of the human RAC1 protein encoded by this human RAC1 gene is shown in SEQ ID NO: 94. In the present specification, RAC1 is not limited to a protein consisting of the amino acid sequence of SEQ ID NO: 94 encoded by the nucleotide sequence of SEQ ID NO: 93. As for RAC1, sequence information has been registered with a plurality of accession numbers as mentioned above, and a plurality of transcript variants are present. The nucleotide sequence of SEQ ID NO: 93 represents the nucleotide sequence of one of these transcript variants.

AKT3, v-akt murine thymoma viral oncogene homolog 3, is also called PKB and known as a member of the serine-threonine protein kinase family AKT. AKT kinases are known as regulatory factors in the cell signaling system that responds to insulin and growth factors. These kinases are involved in various biological processes including glycogen synthesis and glucose uptake as long as cell growth, differentiation, apoptosis, and tumorigenesis. This kinase has been found to be stimulated by platelet-derived growth factor (PDGF), insulin, and insulin-like growth factor 1 (IGF1). AKT3 is present in various animals including humans, and its sequence information is also publicly known (e.g., human: NM_181690.2, NM_001206729.1, NM_005465.4, XM_005272995.2, XM_011544011.1, XM_005272994.3, XM_005272997.3, XM_011544014.1, XM_011544012.1, XM_011544013.1, XM_006711726.2 (NP_859029.1, NP_001193658.1, NP_005456.1, XP_005273052.1, XP_011542313.1, XP_005273051.1, XP_005273054.1, XP_011542316.1, XP_011542314.1, XP_011542315.1, XP_006711789.1), etc.; the numbers represent accession numbers of the NCBI database, and the nucleotide sequence and the amino acid sequence are indicated outside and inside the parentheses, respectively). As one example, the nucleotide sequence of the human AKT3 gene registered in the database as NM_181690.2 is shown in SEQ ID NO: 95, and the amino acid sequence of the human AKT3 protein encoded by this human AKT3 gene is shown in SEQ ID NO: 96. In the present specification, AKT3 is not limited to a protein consisting of the amino acid sequence of SEQ ID NO: 96 encoded by the nucleotide sequence of SEQ ID NO: 95. As for AKT3, sequence information has been registered with a plurality of accession numbers as mentioned above, and a plurality of transcript variants are present. The nucleotide sequence of SEQ ID NO: 95 represents the nucleotide sequence of one of these transcript variants.

EIF4B, eukaryotic translation initiation factor 4B, is known as a protein that is involved in the PI3K-Akt signaling pathway and signals by GPCR. EIF4B is necessary for the binding of mRNA to ribosome and has functions closely related to EIF4-F and EIF4-A. EIF4B is known to bind to or near the 5′-terminal cap of mRNA in the presence of EIF4-F and ATP to stimulate ATPase activity and to stimulate the ATP-dependent RNA unwinding activity of EIF4-A and EIF4-F. EIF4B is present in various animals including humans, and its sequence information is also publicly known (e.g., human: NM_001300821.1, NM_001417.5, XM_006719274.1 (NP_001287750.1, NP_001408.2, XP_006719337.1), etc.; the numbers represent accession numbers of the NCBI database, and the nucleotide sequence and the amino acid sequence are indicated outside and inside the parentheses, respectively). As one example, the nucleotide sequence of the human EIF4B gene registered in the database as NM_001300821.1 is shown in SEQ ID NO: 97, and the amino acid sequence of the human EIF4B protein encoded by this human EIF4B gene is shown in SEQ ID NO: 98. In the present specification, EIF4B is not limited to a protein consisting of the amino acid sequence of SEQ ID NO: 98 encoded by the nucleotide sequence of SEQ ID NO: 97. As for EIF4B, sequence information has been registered with a plurality of accession numbers as mentioned above, and a plurality of transcript variants are present. The nucleotide sequence of SEQ ID NO: 97 represents the nucleotide sequence of one of these transcript variants.

EIF4E, eukaryotic translation initiation factor 4E, is known as a protein that constitutes a eukaryotic translation initiation factor 4F complex, which recognizes the 5′-terminal 7-methylguanosine cap structure of mRNA. EIF4E aids in translation initiation by recruiting ribosomes to the 5′-terminal cap structure. The binding of EIF4E to the 4F complex becomes a rate-limiting stage in translation initiation. EIF4E is present in various animals including humans, and its sequence information is also publicly known (e.g., human: NM_001968.3, NM_001130679.1, NM_001130678.1, XM_006714126.2, XM_006714127.2 (NP_001959.1, NP_001124151.1, NP_001124150.1, XP_006714189.1, XP_006714190.1), etc.; the numbers represent accession numbers of the NCBI database, and the nucleotide sequence and the amino acid sequence are indicated outside and inside the parentheses, respectively). As one example, the nucleotide sequence of the human EIF4E gene registered in the database as NM_001968.3 is shown in SEQ ID NO: 99, and the amino acid sequence of the human EIF4E protein encoded by this human EIF4E gene is shown in SEQ ID NO: 100. In the present specification, EIF4E is not limited to a protein consisting of the amino acid sequence of SEQ ID NO: 100 encoded by the nucleotide sequence of SEQ ID NO: 99. As for EIF4E, sequence information has been registered with a plurality of accession numbers as mentioned above, and a plurality of transcript variants are present. The nucleotide sequence of SEQ ID NO: 99 represents the nucleotide sequence of one of these transcript variants.

ILK, integrin linked kinase, is a protein having a kinase-like domain and four ankyrin-like repeats and is known to control integrin-mediated signal transduction by binding to the cytoplasmic domain of β integrin in the cell membrane. ILK is present in various animals including humans, and its sequence information is also publicly known (e.g., human: NM_004517.3, NM_001278441.1, NM_001014794.2, NM_001278442.1, NM_001014795.2, XM_005252904.3, XM_005252905.1, XM_011520065.1 (NP_004508.1, NP_001265370.1, NP_001014794.1, NP_001265371.1, NP_001014795.1, XP_005252961.1, XP_005252962.1, XP_011518367.1), etc.; the numbers represent accession numbers of the NCBI database, and the nucleotide sequence and the amino acid sequence are indicated outside and inside the parentheses, respectively). As one example, the nucleotide sequence of the human ILK gene registered in the database as NM_004517.3 is shown in SEQ ID NO: 101, and the amino acid sequence of the human ILK protein encoded by this human ILK gene is shown in SEQ ID NO: 102. In the present specification, ILK is not limited to a protein consisting of the amino acid sequence of SEQ ID NO: 102 encoded by the nucleotide sequence of SEQ ID NO: 101. As for ILK, sequence information has been registered with a plurality of accession numbers as mentioned above, and a plurality of transcript variants are present. The nucleotide sequence of SEQ ID NO: 101 represents the nucleotide sequence of one of these transcript variants.

MTCP1, mature T-cell proliferation 1, was identified, as is known, by involvement in some t(X; 14) translocations associated with the growth of mature T cells. This region has a complicated structure with a common promoter and 5′ exons spliced to two different sets of 3′ exons that encode two different proteins. The upstream 13 kD protein is considered to belong to the TCL1 family and participate in leukemia induction. MTCP1 is present in various animals including humans, and its sequence information is also publicly known (e.g., human: NM_001018025.3 (NP_001018025.1), etc.; the numbers represent accession numbers of the NCBI database, and the nucleotide sequence and the amino acid sequence are indicated outside and inside the parentheses, respectively). As one example, the nucleotide sequence of the human MTCP1 gene registered in the database as NM_001018025.3 is shown in SEQ ID NO: 103, and the amino acid sequence of the human MTCP1 protein encoded by this human MTCP1 gene is shown in SEQ ID NO: 104. In the present specification, MTCP1 is not limited to a protein consisting of the amino acid sequence of SEQ ID NO: 104 encoded by the nucleotide sequence of SEQ ID NO: 103. As for MTCP1, there is the possibility that a plurality of transcript variants are present. The nucleotide sequence of SEQ ID NO: 103 represents the nucleotide sequence of one of these transcript variants.

PIK3CA, phosphatidylinositol-4,5-bisphosphate 3-kinase, catalytic subunit alpha, is a 110 kD catalytic subunit in phosphatidylinositol 3-kinase and phosphorylates PtdIns, PtdIns4P, and PtdIns(4,5)P2 by use of ATP. PIK3CA is present in various animals including humans, and its sequence information is also publicly known (e.g., human: NM_006218.2, XM_006713658.2, XM_011512894.1 (NP_006209.2, XP_006713721.1, XP_011511196.1), etc.; the numbers represent accession numbers of the NCBI database, and the nucleotide sequence and the amino acid sequence are indicated outside and inside the parentheses, respectively). As one example, the nucleotide sequence of the human PIK3CA gene registered in the database as NM_006218.2 is shown in SEQ ID NO: 105, and the amino acid sequence of the human PIK3CA protein encoded by this human PIK3CA gene is shown in SEQ ID NO: 106. In the present specification, PIK3CA is not limited to a protein consisting of the amino acid sequence of SEQ ID NO: 106 encoded by the nucleotide sequence of SEQ ID NO: 105. As for PIK3CA, sequence information has been registered with a plurality of accession numbers as mentioned above, and a plurality of transcript variants are present. The nucleotide sequence of SEQ ID NO: 105 represents the nucleotide sequence of one of these transcript variants.

SRF, serum response factor, is known as a ubiquitous nuclear protein that stimulates cell growth and differentiation. SRF belongs to the transcription factor MADS (MCM1, Agamous, Deficiens, and SRF) box superfamily. SRF binds to a serum response element (SRE) in the promoter region of a target gene. SRF regulates the activation of many immediate early genes such as c-fos and thereby participates in cell cycle regulation, apoptosis, cell growth, and cell differentiation. SRF is present in various animals including humans, and its sequence information is also publicly known (e.g., human: NM_003131.3, NM_001292001.1 (NP_003122.1, NP_001278930.1), etc.; the numbers represent accession numbers of the NCBI database, and the nucleotide sequence and the amino acid sequence are indicated outside and inside the parentheses, respectively). As one example, the nucleotide sequence of the human SRF gene registered in the database as NM_003131.3 is shown in SEQ ID NO: 107, and the amino acid sequence of the human SRF protein encoded by this human SRF gene is shown in SEQ ID NO: 108. In the present specification, SRF is not limited to a protein consisting of the amino acid sequence of SEQ ID NO: 108 encoded by the nucleotide sequence of SEQ ID NO: 107. As for SRF, sequence information has been registered with a plurality of accession numbers as mentioned above, and a plurality of transcript variants are present. The nucleotide sequence of SEQ ID NO: 107 represents the nucleotide sequence of one of these transcript variants.

Although GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF can be identified by specific nucleotide sequences and amino acid sequences as mentioned above, the possibility must be taken into consideration that mutations occur in the nucleotide sequences or the amino acid sequences among organism individuals.

Specifically, GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF are not limited to proteins having the same sequences as the amino acid sequences registered in the database and include proteins that have sequences differing from these sequences by 1 or 2 or more, typically, 1 or several, for example, 1, 2, 3, 4, 5, 6, 7, 8, 9, or 10 amino acids and have functions equivalent to GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF.

Moreover, GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF include ones that consist of nucleotide sequences having 70% or higher, 80% or higher, 90% or higher, 95% or higher, or 97% or higher identity to the specific nucleotide sequences mentioned above and encode proteins having functions equivalent to GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF.

The specific functions of GST-π, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, B FAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF are as mentioned above.

In the present specification, the phrases such as “as used herein”, “used herein”, “in the present specification”, and “described herein” mean that the description subsequent thereto is applied to all aspects of the invention described in the present specification, unless otherwise specified. Also, all technical terms and scientific terms used herein have the same meanings as those commonly understood by those skilled in the art unless otherwise defined. All patents, bulletins, and other publications cited herein are incorporated herein by reference in their entirety.

The “drug inhibiting GST-π” used herein is not limited and includes, for example, drugs inhibiting the production and/or activity of GST-π and drugs promoting the degradation and/or deactivation of GST-π. Examples of the drug inhibiting the production of GST-π include, but are not limited to, RNAi molecules against DNA encoding GST-π, ribozymes, antisense nucleic acids, DNA/RNA chimeric polynucleotides, and vectors for expressing them.

Alternatively, any compound that acts on GST-π can be used as the drug inhibiting GST-π. An organic compound (an amino acid, a polypeptide or a derivative thereof, a low-molecular-weight compound, a sugar, a high-molecular-weight compound, etc.), an inorganic compound, or the like can be used as such a compound. Also, such a compound may be any of natural and nonnatural substances. Examples of the derivative of the polypeptide include modified polypeptides obtained by adding modifying groups, and variant polypeptides obtained by altering amino acid residues. In addition, such a compound may be a single compound or may be a compound library, an expression product of a gene library, a cell extract, a cell culture supernatant, a product by a fermentation microorganism, a marine organism extract, a plant extract, or the like. That is, the “drug inhibiting GST-π” is not limited to nucleic acids such as RNAi molecules and includes any compound.

Specifically, examples of the drug inhibiting the activity of GST-π include, but are not limited to, substances binding to GST-π, for example, glutathione, glutathione analogs (e.g., those described in WO 95/08563, WO 96/40205, WO 99/54346, Non Patent Literature 4, etc.), ketoprofen (Non Patent Literature 2), indomethacin (Hall et al., Cancer Res. 1989; 49 (22): 6265-8), ethacrynic acid, piriprost (Tew et al., Cancer Res. 1988; 48 (13): 3622-5), anti-GST-π antibodies, and dominant negative mutants of GST-π. These drugs are commercially available or can be appropriately produced on the basis of publicly known techniques.

RNAi molecules against DNA encoding GST-π, ribozymes, antisense nucleic acids, DNA/RNA chimeric polynucleotides, and vectors for expressing them are preferable as the drug inhibiting the production or activity of GST-π because of high specificity and a low possibility of adverse reactions.

The inhibition of GST-π can be determined when the expression or activity of GST-π in the cell is inhibited as compared with the case the GST-π-inhibiting agent is not allowed to act thereon. The expression of GST-π can be evaluated without limitations by a known arbitrary approach, for example, an immunoprecipitation method using an anti-GST-π antibody, EIA, ELISA, IRA, IRMA, Western blotting, an immunohistochemical method, an immunocytochemical method, flow cytometry, or various hybridization methods, Northern blotting, Southern blotting, or various PCR methods which employ nucleic acids specifically hybridizing to a nucleic acid encoding GST-π or a unique fragment thereof, or a transcript (e.g., mRNA) or a splicing product of the nucleic acid.

Also, the activity of GST-π can be evaluated without limitations by analyzing the known activity of GST-π, for example, binding activity against a protein such as Raf-1 (particularly, phosphorylated Raf-1) or EGFR (particularly, phosphorylated EGFR) by a known arbitrary method, for example, an immunoprecipitation method, Western blotting, mass spectrometry, a pull-down method, or a surface plasmon resonance (SPR) method.

The “drug inhibiting a homeostasis-related protein that exhibits synthetic lethality when inhibited together with GST-π” used herein is not limited and includes, for example, drugs inhibiting the production and/or activity of the protein and drugs promoting the degradation and/or deactivation of the protein. Examples of the drug inhibiting the production of the protein include, but are not limited to, RNAi molecules against DNA encoding the homeostasis-related protein, ribozymes, antisense nucleic acids, DNA/RNA chimeric polynucleotides, and vectors for expressing them. Alternatively, any compound that acts on the protein can be used as the drug inhibiting the activity of the homeostasis-related protein or the drug promoting the degradation and/or deactivation of the homeostasis-related protein. An organic compound (an amino acid, a polypeptide or a derivative thereof, a low-molecular-weight compound, a sugar, a high-molecular-weight compound, etc.), an inorganic compound, or the like can be used as such a compound. Also, such a compound may be any of natural and nonnatural substances. Examples of the derivative of the polypeptide include modified polypeptides obtained by adding modifying groups, and variant polypeptides obtained by altering amino acid residues. In addition, such a compound may be a single compound or may be a compound library, an expression product of a gene library, a cell extract, a cell culture supernatant, a product by a fermentation microorganism, a marine organism extract, a plant extract, or the like. That is, the “drug inhibiting a homeostasis-related protein that exhibits synthetic lethality when inhibited together with GST-π” is not limited to nucleic acids such as RNAi molecules and includes any compound.

More specifically, examples of the drug inhibiting the activity of p21 among the cell cycle-regulating proteins that exhibit synthetic lethality when inhibited together with GST-π include, but are not limited to: butyrolactone I (Sax et al., Cell Cycle, January; 1 (1): 90-6, 2002), which is a low-molecular-weight compound promoting the proteasomal degradation of the p21 protein while also inhibiting the enzymatic activity of CDC2, CDK2, and CDK5; quetiapine (Kondo et al., Transl. Psychiatry, April 2; 3: e243, 2013), which is a psychotropic drug reportedly specifically inhibiting the expression of p21 in the nerve cells or oligodendrocytes of CD-1 mice; Sorafenib (Inoue et al., Cancer Biology & Therapy, 12: 9, 827-836, 2011), which is a low-molecular-weight compound specifically inhibiting p21 without the involvement of p53, p27, or Akt and inhibiting multikinase for Raf, VEGFR, PDGFR, and the like; UC2288 (Wettersten et al., Cancer Biology & Therapy, 14 (3), 278-285, 2013), which is a low-molecular-weight compound specifically inhibiting p21 without the involvement of p53 or Akt; and RNAi molecules against DNA encoding p21, ribozymes, antisense nucleic acids, DNA/RNA chimeric polynucleotides, and vectors for expressing them, anti-p21 antibodies, and dominant negative variants of p21. These drugs are commercially available or can be appropriately produced on the basis of publicly known techniques.

Examples of the drug inhibiting the activity of RNPC1 among the cell cycle-regulating proteins that exhibit synthetic lethality when inhibited together with GST-π include, but are not limited to, RNAi molecules against DNA encoding RNPC1, ribozymes, antisense nucleic acids, DNA/RNA chimeric polynucleotides, and vectors for expressing them, anti-RNPC1 antibodies, and dominant negative variants of RNPC1. These drugs are commercially available or can be appropriately produced on the basis of publicly known techniques.

Examples of the drug inhibiting the activity of CCNL1 among the cell cycle-regulating proteins that exhibit synthetic lethality when inhibited together with GST-π include, but are not limited to, RNAi molecules against DNA encoding CCNL1, ribozymes, antisense nucleic acids, DNA/RNA chimeric polynucleotides, and vectors for expressing them, anti-CCNL1 antibodies, and dominant negative variants of CCNL1. These drugs are commercially available or can be appropriately produced on the basis of publicly known techniques.

Examples of the drug inhibiting the activity of MCM8 among the cell cycle-regulating proteins that exhibit synthetic lethality when inhibited together with GST-π include, but are not limited to, RNAi molecules against DNA encoding MCM8, ribozymes, antisense nucleic acids, DNA/RNA chimeric polynucleotides, and vectors for expressing them, anti-MCM8 antibodies, and dominant negative variants of MCM8. These drugs are commercially available or can be appropriately produced on the basis of publicly known techniques.

Examples of the drug inhibiting the activity of CCNB3 among the cell cycle-regulating proteins that exhibit synthetic lethality when inhibited together with GST-π include, but are not limited to, RNAi molecules against DNA encoding CCNB3, ribozymes, antisense nucleic acids, DNA/RNA chimeric polynucleotides, and vectors for expressing them, anti-CCNB3 antibodies, and dominant negative variants of CCNB3. These drugs are commercially available or can be appropriately produced on the basis of publicly known techniques.

Examples of the drug inhibiting the activity of MCMDC1 among the cell cycle-regulating proteins that exhibit synthetic lethality when inhibited together with GST-π include, but are not limited to, RNAi molecules against DNA encoding MCMDC1, ribozymes, antisense nucleic acids, DNA/RNA chimeric polynucleotides, and vectors for expressing them, anti-MCMDC1 antibodies, and dominant negative variants of MCMDC1. These drugs are commercially available or can be appropriately produced on the basis of publicly known techniques.

Examples of the drug inhibiting the activity of ATM among the cell cycle-regulating proteins that exhibit synthetic lethality when inhibited together with GST-π include, but are not limited to: CGK 733 (Won et al., Nat. Chem. Biol. 2, 369, 2006), which is a low-molecular-weight compound selectively inhibiting the kinase activity of ATM and ATR; KU-55933 (Lau et al., Nat. Cell Biol. 7, 493, 2005), KU-60019 (Zirkin et al., J Biol Chem. July 26; 288 (30): 21770-83, 2013), and CP-466722 (Rainey et al., Cancer Res. September 15; 68 (18):7466-74, 2008), which are low-molecular-weight compounds selectively inhibiting the kinase activity of ATM; and RNAi molecules against DNA encoding ATM, ribozymes, antisense nucleic acids, DNA/RNA chimeric polynucleotides, and vectors for expressing them, anti-ATM antibodies, and dominant negative variants of ATM. These drugs are commercially available or can be appropriately produced on the basis of publicly known techniques.

Examples of the drug inhibiting the activity of CDC25A among the cell cycle-regulating proteins that exhibit synthetic lethality when inhibited together with GST-π include, but are not limited to: NSC95397 (Lazo J S et al., Mol. Pharmacol. 61: 720-728, 2002), which is a low-molecular-weight compound inhibiting the phosphatase activity of each of human CDC25A, human CDC25B, and human CDC25C; SC alpha alpha delta 09 (Rice, R. L. et al., Biochemistry 36 (50): 15965-15974, 1997), which is a low-molecular-weight compound inhibiting the phosphatase activity of each of human CDC25A, human CDC25B, human CDC25C, and human tyrosine phosphatase PTB1B; and RNAi molecules against DNA encoding CDC25A, ribozymes, antisense nucleic acids, DNA/RNA chimeric polynucleotides, and vectors for expressing them, anti-CDC25A antibodies, and dominant negative variants of CDC25A. These drugs are commercially available or can be appropriately produced on the basis of publicly known techniques.

Examples of the drug inhibiting the activity of PRKDC among the cell cycle-regulating proteins that exhibit synthetic lethality when inhibited together with GST-π include, but are not limited to, RNAi molecules against DNA encoding PRKDC, ribozymes, antisense nucleic acids, DNA/RNA chimeric polynucleotides, and vectors for expressing them, anti-PRKDC antibodies, and dominant negative variants of PRKDC. These drugs are commercially available or can be appropriately produced on the basis of publicly known techniques.

Examples of the drug inhibiting the activity of RBBP8 among the cell cycle-regulating proteins that exhibit synthetic lethality when inhibited together with GST-π include, but are not limited to, RNAi molecules against DNA encoding RBBP8, ribozymes, antisense nucleic acids, DNA/RNA chimeric polynucleotides, and vectors for expressing them, anti-RBBP8 antibodies, and dominant negative variants of RBBP8. These drugs are commercially available or can be appropriately produced on the basis of publicly known techniques.

Examples of the drug inhibiting the activity of SKP2 among the cell cycle-regulating proteins that exhibit synthetic lethality when inhibited together with GST-π include, but are not limited to, RNAi molecules against DNA encoding SKP2, ribozymes, antisense nucleic acids, DNA/RNA chimeric polynucleotides, and vectors for expressing them, anti-SKP2 antibodies, and dominant negative variants of SKP2. These drugs are commercially available or can be appropriately produced on the basis of publicly known techniques.

Examples of the drug inhibiting the activity of MCM10 among the cell cycle-regulating proteins that exhibit synthetic lethality when inhibited together with GST-π include, but are not limited to, RNAi molecules against DNA encoding MCM10, ribozymes, antisense nucleic acids, DNA/RNA chimeric polynucleotides, and vectors for expressing them, anti-MCM10 antibodies, and dominant negative variants of MCM10. These drugs are commercially available or can be appropriately produced on the basis of publicly known techniques.

Examples of the drug inhibiting the activity of CENPH among the cell cycle-regulating proteins that exhibit synthetic lethality when inhibited together with GST-π include, but are not limited to, RNAi molecules against DNA encoding CENPH, ribozymes, antisense nucleic acids, DNA/RNA chimeric polynucleotides, and vectors for expressing them, anti-CENPH antibodies, and dominant negative variants of CENPH. These drugs are commercially available or can be appropriately produced on the basis of publicly known techniques.

Examples of the drug inhibiting the activity of BRSK1 among the cell cycle-regulating proteins that exhibit synthetic lethality when inhibited together with GST-π include, but are not limited to, RNAi molecules against DNA encoding BRSK1, ribozymes, antisense nucleic acids, DNA/RNA chimeric polynucleotides, and vectors for expressing them, anti-BRSK1 antibodies, and dominant negative variants of BRSK1. These drugs are commercially available or can be appropriately produced on the basis of publicly known techniques.

Examples of the drug inhibiting the activity of MYLK among the cell cycle-regulating proteins that exhibit synthetic lethality when inhibited together with GST-π include, but are not limited to, HA-100 Dihydrochloride (Gerard et al., J Clin Invest. January; 77 (1): 61-5. 1986), which is a low-molecular-weight compound inhibiting the activity of MYLK in addition to the inhibition of the activity of various protein kinases (PKA and PKC, etc.); Staurosporine (Tamaoki et al., Biochem. Biophys. Res. Commun. 135: 397-402. 1986), which is a low-molecular-weight compound inhibiting the activity of MYLK in addition to the inhibition of the activity of various protein kinases (PKA, PKC, PKG, CaMK, CaMKII, and phosphorylase kinase, etc.); Calphostin C (Kobayashi et al., Biochem. Biophys. Res. Commun. 159: 548-553. 1989), which is a low-molecular-weight compound serving as a selective inhibitor of PKC and also inhibiting the activity of MYLK; Piceatannol (Oliver et al., J. Biol. Chem. 269: 29697-29703. 1994), which is a low-molecular-weight compound serving as a tyrosine kinase inhibitor and also inhibiting the activity of MYLK; A-3 Hydrochloride (Inagaki et al., Mol. Pharmacol. 29, 577. 1986), which is a low-molecular-weight compound inhibiting the activity of MYLK in addition to the inhibition of the activity of various casein kinases and protein kinases; H-7 Dihydrochloride (Kawamoto et al., Biochem. Biophys. Res. Commun. 125: 258-264. 1984), which is a low-molecular-weight compound serving as a cell-permeable serine/threonine kinase inhibitor and also inhibiting the activity of MYLK; H-9 Hydrochloride (Wolf et al., J. Biol. Chem. 260: 15718-15722. 1985), which is a low-molecular-weight compound inhibiting the activity of MYLK in addition to the inhibition of the activity of various protein kinases (PKA and PKC, etc.); W-5 (Hidaka et al., Mol. Pharmacol. 20: 571-578. 1981), which is a low-molecular-weight compound serving as a calmodulin (CaM) antagonist and also inhibiting the activity of MYLK; W-7 (Hidaka et al., Proc. Natl. Acad. Sci. U.S.A. 78: 4354-4357. 1981), which is a low-molecular-weight compound serving as a calmodulin (CaM) antagonist and also inhibiting the activity of MYLK; W-13 Isomer Hydrochloride (Hidaka et al., Proc. Natl. Acad. Sci. U.S.A. 78: 4354-4357. 1981), which is a low-molecular-weight compound inhibiting the activity of MYLK in addition to the inhibition of the activity of various protein kinases (PKA and PKC, etc.); ML-7 Dihydrochloride (Saitoh et al., J. Biol. Chem. 262: 7796-7801. 1987), which is a low-molecular-weight compound inhibiting the activity of MYLK in addition to the inhibition of the activity of various protein kinases (PKA and PKC, etc.); ML-9 (Saitoh et al., Biochem. Biophys. Res. Commun. 140: 280-287. 1986), which is a low-molecular-weight compound selectively inhibiting MYLK and CaMK; Myricetin (Hagiwara et al., Biochem. Pharmacol. 37: 2987-2992. 1988), which is a low-molecular-weight compound inhibiting the activity of MYLK in addition to the inhibition of the activity of various casein kinases and protein kinases; E6 Berbamine (Hu et al., Biochem. Pharmacol. October 20; 44 (8): 1543-7. 1992), which is a low-molecular-weight compound serving as a cell-permeable calmodulin (CaM) antagonist and also inhibiting the activity of MYLK; K-252a (Kase et al., J. Antibiot. 39: 1059-1065. 1986), which is a low-molecular-weight compound inhibiting the activity of MYLK in addition to the inhibition of the activity of various protein kinases; K-252b (Nakanishi et al., J. Antibiot. 39: 1066-1071. 1986), which is a low-molecular-weight compound inhibiting the activity of MYLK in addition to the inhibition of the activity of various protein kinases; Altenusin (Rosett et al., Biochem. J. 67: 390-400. 1957), which is a low-molecular-weight compound serving as a c-Src inhibitor and also inhibiting the activity of MYLK; and RNAi molecules against DNA encoding MYLK, ribozymes, antisense nucleic acids, DNA/RNA chimeric polynucleotides, and vectors for expressing them, anti-MYLK antibodies, and dominant negative variants of MYLK. These drugs are commercially available or can be appropriately produced on the basis of publicly known techniques.

On the other hand, examples of the drug inhibiting the activity of AATF among the anti-apoptosis-related proteins that exhibit synthetic lethality when inhibited together with GST-π include, but are not limited to, RNAi molecules against DNA encoding AATF, ribozymes, antisense nucleic acids, DNA/RNA chimeric polynucleotides, and vectors for expressing them, anti-AATF antibodies, and dominant negative variants of AATF. These drugs are commercially available or can be appropriately produced on the basis of publicly known techniques.

Examples of the drug inhibiting the activity of ALOX12 among the anti-apoptosis-related proteins that exhibit synthetic lethality when inhibited together with GST-π include, but are not limited to, RNAi molecules against DNA encoding ALOX12, ribozymes, antisense nucleic acids, DNA/RNA chimeric polynucleotides, and vectors for expressing them, anti-ALOX12 antibodies, and dominant negative variants of ALOX12. These drugs are commercially available or can be appropriately produced on the basis of publicly known techniques.

Examples of the drug inhibiting the activity of ANXA1 among the anti-apoptosis-related proteins that exhibit synthetic lethality when inhibited together with GST-π include, but are not limited to, RNAi molecules against DNA encoding ANXA1, ribozymes, antisense nucleic acids, DNA/RNA chimeric polynucleotides, and vectors for expressing them, anti-ANXA1 antibodies, and dominant negative variants of ANXA1. These drugs are commercially available or can be appropriately produced on the basis of publicly known techniques.

Examples of the drug inhibiting the activity of ANXA4 among the anti-apoptosis-related proteins that exhibit synthetic lethality when inhibited together with GST-π include, but are not limited to, RNAi molecules against DNA encoding ANXA4, ribozymes, antisense nucleic acids, DNA/RNA chimeric polynucleotides, and vectors for expressing them, anti-ANXA4 antibodies, and dominant negative variants of ANXA4. These drugs are commercially available or can be appropriately produced on the basis of publicly known techniques.

Examples of the drug inhibiting the activity of API5 among the anti-apoptosis-related proteins that exhibit synthetic lethality when inhibited together with GST-π include, but are not limited to, RNAi molecules against DNA encoding API5, ribozymes, antisense nucleic acids, DNA/RNA chimeric polynucleotides, and vectors for expressing them, anti-API5 antibodies, and dominant negative variants of API5. These drugs are commercially available or can be appropriately produced on the basis of publicly known techniques.

Examples of the drug inhibiting the activity of ATF5 among the anti-apoptosis-related proteins that exhibit synthetic lethality when inhibited together with GST-π include, but are not limited to, RNAi molecules against DNA encoding ATF5, ribozymes, antisense nucleic acids, DNA/RNA chimeric polynucleotides, and vectors for expressing them, anti-ATF5 antibodies, and dominant negative variants of ATF5. These drugs are commercially available or can be appropriately produced on the basis of publicly known techniques.

Examples of the drug inhibiting the activity of AVEN among the anti-apoptosis-related proteins that exhibit synthetic lethality when inhibited together with GST-π include, but are not limited to, RNAi molecules against DNA encoding AVEN, ribozymes, antisense nucleic acids, DNA/RNA chimeric polynucleotides, and vectors for expressing them, anti-AVEN antibodies, and dominant negative variants of AVEN. These drugs are commercially available or can be appropriately produced on the basis of publicly known techniques.

Examples of the drug inhibiting the activity of AZU1 among the anti-apoptosis-related proteins that exhibit synthetic lethality when inhibited together with GST-π include, but are not limited to, RNAi molecules against DNA encoding AZU1, ribozymes, antisense nucleic acids, DNA/RNA chimeric polynucleotides, and vectors for expressing them, anti-AZU1 antibodies, and dominant negative variants of AZU1. These drugs are commercially available or can be appropriately produced on the basis of publicly known techniques.

Examples of the drug inhibiting the activity of BAG1 among the anti-apoptosis-related proteins that exhibit synthetic lethality when inhibited together with GST-π include, but are not limited to, RNAi molecules against DNA encoding BAG1, ribozymes, antisense nucleic acids, DNA/RNA chimeric polynucleotides, and vectors for expressing them, anti-BAG1 antibodies, and dominant negative variants of BAG1. These drugs are commercially available or can be appropriately produced on the basis of publicly known techniques.

Examples of the drug inhibiting the activity of BCL2L1 among the anti-apoptosis-related proteins that exhibit synthetic lethality when inhibited together with GST-π include, but are not limited to, RNAi molecules against DNA encoding BCL2L1, ribozymes, antisense nucleic acids, DNA/RNA chimeric polynucleotides, and vectors for expressing them, anti-BCL2L1 antibodies, and dominant negative variants of BCL2L1. These drugs are commercially available or can be appropriately produced on the basis of publicly known techniques.

Examples of the drug inhibiting the activity of BFAR among the anti-apoptosis-related proteins that exhibit synthetic lethality when inhibited together with GST-π include, but are not limited to, RNAi molecules against DNA encoding BFAR, ribozymes, antisense nucleic acids, DNA/RNA chimeric polynucleotides, and vectors for expressing them, anti-BFAR antibodies, and dominant negative variants of BFAR. These drugs are commercially available or can be appropriately produced on the basis of publicly known techniques.

Examples of the drug inhibiting the activity of CFLAR among the anti-apoptosis-related proteins that exhibit synthetic lethality when inhibited together with GST-π include, but are not limited to, RNAi molecules against DNA encoding CFLAR, ribozymes, antisense nucleic acids, DNA/RNA chimeric polynucleotides, and vectors for expressing them, anti-CFLAR antibodies, and dominant negative variants of CFLAR. These drugs are commercially available or can be appropriately produced on the basis of publicly known techniques.

Examples of the drug inhibiting the activity of IL2 among the anti-apoptosis-related proteins that exhibit synthetic lethality when inhibited together with GST-π include, but are not limited to, RNAi molecules against DNA encoding IL2, ribozymes, antisense nucleic acids, DNA/RNA chimeric polynucleotides, and vectors for expressing them, anti-IL2 antibodies, and dominant negative variants of IL2. These drugs are commercially available or can be appropriately produced on the basis of publicly known techniques.

Examples of the drug inhibiting the activity of MALT1 among the anti-apoptosis-related proteins that exhibit synthetic lethality when inhibited together with GST-π include, but are not limited to, RNAi molecules against DNA encoding MALT1, ribozymes, antisense nucleic acids, DNA/RNA chimeric polynucleotides, and vectors for expressing them, anti-MALT1 antibodies, and dominant negative variants of MALT1. These drugs are commercially available or can be appropriately produced on the basis of publicly known techniques.

Examples of the drug inhibiting the activity of MCL1 among the anti-apoptosis-related proteins that exhibit synthetic lethality when inhibited together with GST-π include, but are not limited to, Synribo (omacetaxine mepesuccinate), which is approved as a therapeutic agent for chronic myelocytic leukemia, RNAi molecules against DNA encoding MCL1, ribozymes, antisense nucleic acids, DNA/RNA chimeric polynucleotides, and vectors for expressing them, anti-MCL1 antibodies, and dominant negative variants of MCL1. These drugs are commercially available or can be appropriately produced on the basis of publicly known techniques.

Examples of the drug inhibiting the activity of MKL1 among the anti-apoptosis-related proteins that exhibit synthetic lethality when inhibited together with GST-π include, but are not limited to, RNAi molecules against DNA encoding MKL1, ribozymes, antisense nucleic acids, DNA/RNA chimeric polynucleotides, and vectors for expressing them, anti-MKL1 antibodies, and dominant negative variants of MKL1. These drugs are commercially available or can be appropriately produced on the basis of publicly known techniques.

Examples of the drug inhibiting the activity of MPO among the anti-apoptosis-related proteins that exhibit synthetic lethality when inhibited together with GST-π include, but are not limited to, RNAi molecules against DNA encoding MPO, ribozymes, antisense nucleic acids, DNA/RNA chimeric polynucleotides, and vectors for expressing them, anti-MPO antibodies, and dominant negative variants of MPO. These drugs are commercially available or can be appropriately produced on the basis of publicly known techniques.

Examples of the drug inhibiting the activity of MTL5 among the anti-apoptosis-related proteins that exhibit synthetic lethality when inhibited together with GST-π include, but are not limited to, RNAi molecules against DNA encoding MTL5, ribozymes, antisense nucleic acids, DNA/RNA chimeric polynucleotides, and vectors for expressing them, anti-MTL5 antibodies, and dominant negative variants of MTL5. These drugs are commercially available or can be appropriately produced on the basis of publicly known techniques.

Examples of the drug inhibiting the activity of MYBL2 among the anti-apoptosis-related proteins that exhibit synthetic lethality when inhibited together with GST-π include, but are not limited to, RNAi molecules against DNA encoding MYBL2, ribozymes, antisense nucleic acids, DNA/RNA chimeric polynucleotides, and vectors for expressing them, anti-MYBL2 antibodies, and dominant negative variants of MYBL2. These drugs are commercially available or can be appropriately produced on the basis of publicly known techniques.

Examples of the drug inhibiting the activity of MYO18A among the anti-apoptosis-related proteins that exhibit synthetic lethality when inhibited together with GST-π include, but are not limited to, RNAi molecules against DNA encoding MYO18A, ribozymes, antisense nucleic acids, DNA/RNA chimeric polynucleotides, and vectors for expressing them, anti-MYO18A antibodies, and dominant negative variants of MYO18A. These drugs are commercially available or can be appropriately produced on the basis of publicly known techniques.

On the other hand, examples of the drug inhibiting the activity of MTOR among the PI3K signaling pathway-related proteins that exhibit synthetic lethality when inhibited together with GST-π include, but are not limited to, Rapamycin (Chang et al., Trends Pharmacol. Sci. 12: 218-223. 1991), which is a low-molecular-weight compound serving as a macrolide immunosuppressant and inhibiting the activity of MTOR; Everolimus (Weisblum et al., Br. Med. Bull. 40: 47-53. 1984), which is a low-molecular-weight compound serving as a rapamycin-derived macrolide immunosuppressant and inhibiting the activity of MTOR; BEZ235 (Maira et al., Mol. Cancer Ther. 7 (7): 1851-1863. 2008), which is a low-molecular-weight compound serving as a PI3K tyrosine kinase inhibitor and inhibiting the activity of MTOR; AZD8055 (Huang et al., J Biol Chem. November 18; 286 (46): 40002-12. 2011), which is a low-molecular-weight compound selectively inhibiting the kinase activity of MTOR; PI-103 (Demyanets et al., 2010. Basic Res Cardiol. March; 106 (2): 217-31. 2011), which is a pyridinylfuranopyrimidine compound and is a low-molecular-weight compound inhibiting the kinase activity of DNA-PK, PI3K, and MTOR; and Niclosamide (Balgi et al., PLoS One. September 22; 4 (9): e7124. 2009), PP242 (Bao et al. J. Cell Biol. 210 (7): 1153-64. 2015), Timosaponin AIII (King et.al, PLoS One. September 30; 4 (9):e7283 2009), KU 0063794 (Garcia-Martinez et al. Biochem. J. 421 (1): 29-42. 2009), AZD2014 (Guichard et al. Mol. Cancer. Ther. 14 (11): 2508-18. 2015), Temsirolimus (Raymond et al. J. Clin. Oncol. 22: 2336-2347. 2004), Palomid 529 (Xue et al. Cancer Res. 68 (22): 9551-7. 2008), Fisetin (Suh et.al, Carcinogenesis. August; 31 (8):1424-33 2010), GDC-0980 (Wallin et al. Mol. Cancer Ther. 10 (12): 2426-36. 2011), SF1126 (Garlich et al. Cancer Res. 68 (1): 206-15. 2008), CH5132799 (Tanaka et al. Clin Cancer Res. 17 (10): 3272-81. 2011), WYE-354 (Yu et al. Cancer Res. 69 (15): 6232-40. 2009), Compound 401 (Ballou et al. J. Biol. Chem. 282, 24463. 2007), Ridaforolimus (Gadducci et al.: Gynecol. Endocrinol. 24, 239. 2008), GSK 1059615 (Steven et al. ACS. Med. Chem. Lett. 1 (1): 39-43 2010), PF-04691502 (Yuan et al. Mol. Cancer Ther. 10 (11): 2189-99. 2011), PP121 (Apsel et al. Nat. Chem. Biol. 4 (11): 691-9. 2008), OSI-027 (Bhaqwat et al. Mol. Cancer Ther. 10 (8): 1394-406. 2011), WYE-125132 (Yu et al. Cancer Res. 70 (2): 621-31. 2010), Umirolimus (Baldo et al. 2008. Curr. Cancer Drug Targets. 8 (8): 647-65. 2008), WAY-600 (Yu et al. Cancer Res. 69 (15): 6232-40. 2009), WYE-687 (Yu et al. Cancer Res. 69 (15): 6232-40. 2009), PKI-179 (Venkatesan et al. Bioorg. Med. Chem. Lett. 20 (19): 5869-73. 2010), PF-05212384 (Akintunde et al. J. Hematol. Oncol. 6: 88. 2013), CAY10626 (Rameh et al. J. Biol. Chem. 274: 8347-8350. 1999), NVP-BGT226 (Chang et al. Clin. Cancer Res. 17 (22): 7116-26. 2011), XL-147 derivative 1 (Akintunde et al. J. Hematol. Oncol. 6: 88. 2013), XL 388 (Eduardo et al. Mol Cancer Ther. 10 (3): 395-403. 2011), Torin 1 (Liu et al. J. Med. Chem. 53 (19): 7146-55. 2010) and RNAi molecules against DNA encoding MTOR, ribozymes, antisense nucleic acids, DNA/RNA chimeric polynucleotides, and vectors for expressing them, anti-MTOR antibodies, and dominant negative variants of MTOR. These drugs are commercially available or can be appropriately produced on the basis of publicly known techniques.

Examples of the drug inhibiting the activity of IRAK1 among the PI3K signaling pathway-related proteins that exhibit synthetic lethality when inhibited together with GST-π include, but are not limited to, Interleukin-1 Receptor-Associated-Kinase-1/4 Inhibitor (Bhattacharyya et al., Am. J. Physiol. Gastrointest. Liver Physiol. 293, G429. 2007), which is a benzimidazole compound and is a low-molecular-weight compound specifically inhibiting the activity of IL-1 kinase; and RNAi molecules against DNA encoding IRAK1, ribozymes, antisense nucleic acids, DNA/RNA chimeric polynucleotides, and vectors for expressing them, anti-IRAK1 antibodies, and dominant negative variants of IRAK1. These drugs are commercially available or can be appropriately produced on the basis of publicly known techniques.

Examples of the drug inhibiting the activity of IRS1 among the PI3K signaling pathway-related proteins that exhibit synthetic lethality when inhibited together with GST-π include, but are not limited to, RNAi molecules against DNA encoding IRS1, ribozymes, antisense nucleic acids, DNA/RNA chimeric polynucleotides, and vectors for expressing them, anti-IRS1 antibodies, and dominant negative variants of IRS1. These drugs are commercially available or can be appropriately produced on the basis of publicly known techniques.

Examples of the drug inhibiting the activity of MYD88 among the PI3K signaling pathway-related proteins that exhibit synthetic lethality when inhibited together with GST-π include, but are not limited to, MyD88 Inhibitory Peptide Pepinh-MYD (Derossi et al., J. Biol. Chem., 269: 10444-50. 1994), which is a MYD88-derived 26-amino acid peptide inhibiting homodimerization; TJ-M2010 (Li et al., Transplant Proc. 45 (5): 1842-5. 2013), which is a low-molecular-weight compound specifically inhibiting MYD88; and RNAi molecules against DNA encoding MYD88, ribozymes, antisense nucleic acids, DNA/RNA chimeric polynucleotides, and vectors for expressing them, anti-MYD88 antibodies, and dominant negative variants of MYD88. These drugs are commercially available or can be appropriately produced on the basis of publicly known techniques.

Examples of the drug inhibiting the activity of NFKB1 among the PI3K signaling pathway-related proteins that exhibit synthetic lethality when inhibited together with GST-π include, but are not limited to, BAY 11-7085 (Pierce et al., J. Biol. Chem. 272: 21096-21103. 1997), which is a low-molecular-weight compound inhibiting the activity of both NF-κB activation and IκBα phosphorylation; Helenalin (Lyss et al., J. Biol. Chem. 273 (50): 33508-16. 1998), which is a low-molecular-weight compound serving as an apoptosis-inducing anticancer agent and inhibiting NF-κB; Caffeic Acid Phenethyl Ester (Sud'ina et al., FEBS Lett. August 23; 329 (1-2): 21-4. 1993), which is a low-molecular-weight compound serving as an inhibitor of HIV integrase and tyrosine kinase and specifically inhibiting the activity of NF-κB; and NFκB Activation Inhibitor II, JSH-23 (Shin et al., FEBS Lett. 571: 50-54. 2004), QNZ (Tobe et al. Bioorg. Med. Chem. 11: 3869-3878. 2003), Andrographolide (Yu et al. Planta Med. December; 69 (12):1075-9. 2003), Curcumin (Asai et al. J. Nutr. 131: 2932-2935. 2001), Aspirin (Kopp et al. Science. 265: 956-959. 1994), Sulfasalazine (Liptay et al. Br. J. Pharmacol. 128, 1361. 1999), Rocaglamide (Engelmeier et al. J. Agric. Food Chem. 48: 1400-1404. 2000), SM 7368 (Lee et al. Biochem. Biophys. Res. Commun. 336: 716-722. 2005), Sulindac Sulfide (Meade et al. J. Biol. Chem. 268: 6610-6614. 1993), Trichodion (Erkel et al. FEBS Lett. 477, 219. 2000), CHS-828 (Hovstadius et al. Clin. Cancer Res. 8 (9): 2843-50. 2002), Z-VRPR-FMK (Hailfinger et al. Proc. Natl. Acad. Sci. U.S.A. 106 (47): 19946-19951. 2009), Sodium Salicylate (Kopp et al. Science. 265: 956-959. 1994), 4-Aminosalicylic Acid (Eberhardt et al. Biochem. Biophys. Res. Commun. 200: 163-170. 1994), Ethyl 3,4-Dihydroxycinnamate (Nakayama et al. Biosci. Biotechnol. Biochem. 60: 316-318. 1996), CAY10512 (Heynekamp et al. J Med Chem 49 7182-7189 2006), N-Stearoyl Phytosphingosine (Ryu et al. Lipids. 45 (7): 613-8. 2010), Palmitic Acid Methyl Ester (Cal et al. Toxicology. 210: 197-204. 2005), 9-Methylstreptimidone (Ishikawa et al. Bioorg. Med. Chem. Lett. 19 (6): 1726-8. 2009), Rocaglaol (Engelmeier et al. J. Agric. Food Chem. 48: 1400-1404. 2000), BAY 11-7082 (Pierce et al. J. Biol. Chem. 272: 21096-21103. 1997), and RNAi molecules against DNA encoding NFKB1, ribozymes, antisense nucleic acids, DNA/RNA chimeric polynucleotides, and vectors for expressing them, anti-NFKB1 antibodies, and dominant negative variants of NFKB1. These drugs are commercially available or can be appropriately produced on the basis of publicly known techniques.

Examples of the drug inhibiting the activity of PIK3CG among the PI3K signaling pathway-related proteins that exhibit synthetic lethality when inhibited together with GST-π include, but are not limited to, CUDC-907 (Qian et al., Clin Cancer Res. 18 (15): 4104-4113. 2012), which is a low-molecular-weight compound specifically inhibiting the activity of both HDAC and PI3K; PKI-402 (Dehnhardt et al., J Med Chem. January 28; 53 (2): 798-810. 2010), which is a low-molecular-weight compound specifically inhibiting the activity of both PI3K and mTOR; PF-04691502 (Yuan et al., Mol Cancer Ther, 10 (11), 2189-2199. 2011), which is a low-molecular-weight compound specifically inhibiting the activity of both PI3K and mTOR; NVP-BGT226 (Glienke et al., Tumor Biology, 33 (3): 757-765. 2012), which is a low-molecular-weight compound specifically inhibiting the activity of both PI3K and mTOR; and IPI-145 (INK1197) (Winkler et al., Chem Biol. 2013 Nov. 21; 20 (11): 1364-74. 2013), SAR245409 (XL765) (Dai et.al, Endocrinology. March; 154 (3):1247-592013), ZSTK474 (Toyama et.al, Arthritis Res Ther. 12 (3): R92. 2010), VS-5584 (SB2343) (Hart et.al, Mol Cancer Ther. 2013 February; 12 (2):151-61. 2013), AS-605240 (Camps et.al, Nature medicine, 11 (9): 936-943. 2005), PIK-90 (Van et.al, J Cell Biol. 2006 Jul. 31; 174 (3):437-45. 2006), PF-4989216 (Walls et.al, Clin Cancer Res. 2014 Feb. 1; 20 (3):631-43. 2014), TG100-115 (Walls et.al, Proc Natl Acad Sci USA. December 26; 103 (52):19866-71. 2006), BKM120 (Bendell et.al, J. Clin. Oncol. 30 (3): 282-90. 2012), BEZ235 Tosylate (Maira et.al, Mol Cancer Ther 2008; 7: 1851-1863. 2008), LY294002 (Maira et.al, Biochem. Soc. Trans. 37 (Pa): 265-72. 2009), PI-103 (Raynaud et.al, Molecular Cancer Therapeutics, 8 (7): 1725-1738. 2009), XL147 (Shapiro et.al, Proc 97th Annu Meet AACR, 14-18. 2007), AS-252424 (Pomel et.al, J Med Chem. June 29; 49 (13):3857-71. 2006), AS-604850 (Camps et.al, Nature medicine, 11 (9): 936-943. 2005), CAY10505 (Tyagi et.al, Can J Physiol Pharmacol. July; 90 (7):881-5. 2012), CH5132799 (Ohwada et.al, Bioorganic & medicinal chemistry letters, 21 (6): 1767-1772. 2011), BAY 80-6946 (Copanlisib) (Patnaik et.al, J Clin Oncol, 29, 2011), GDC-0032 (Ndubaku et.al, J Med Chem. June 13; 56 (11):4597-610. 2013), GSK1059615 (Knight et.al, ACS Medicinal Chemistry Letters, 1 (1): 39-43. 2010), CAL-130 (Subramaniam et.al, Cancer Cell. 21 (4):459-72. 2012), XL765 (Laird et.al, AACR-NCI-EORTC International Conference on Molecular Targets and Cancer Therapeutics. October p. B250 2007), and RNAi molecules against DNA encoding PIK3CG, ribozymes, antisense nucleic acids, DNA/RNA chimeric polynucleotides, and vectors for expressing them, anti-PIK3CG antibodies, and dominant negative variants of PIK3CG. These drugs are commercially available or can be appropriately produced on the basis of publicly known techniques.

Examples of the drug inhibiting the activity of RAC1 among the PI3K signaling pathway-related proteins that exhibit synthetic lethality when inhibited together with GST-π include, but are not limited to, NSC 23766 (Gao et al., Proc. Natl. Acad. Sci. U.S.A. 101: 7618-7623. 2004), which is a low-molecular-weight compound specifically inhibiting the activity of Rac GTPase; W56 (Gao et al., J. Biol. Chem. 276 47530. 2001), which is a peptide derived from guanine nucleotide exchange factor recognition/activation site of RAC1; and RNAi molecules against DNA encoding RAC1, ribozymes, antisense nucleic acids, DNA/RNA chimeric polynucleotides, and vectors for expressing them, anti-RAC1 antibodies, and dominant negative variants of RAC1. These drugs are commercially available or can be appropriately produced on the basis of publicly known techniques.

Examples of the drug inhibiting the activity of AKT3 among the PI3K signaling pathway-related proteins that exhibit synthetic lethality when inhibited together with GST-π include, but are not limited to, MK-2206 Dihydrochloride (Hirai et al., Mol. Cancer ther. 9 (7): 1956-67. 2010), which is a low-molecular-weight compound specifically inhibiting the activity of Akt1, Akt2, and Akt3; Triciribine (Moore et al., Biochem. Pharmacol. 38: 4037-4044. 1989), which is a low-molecular-weight compound specifically inhibiting the activity of Akt; Akt Inhibitor VIII (Barnett et al., Biochem. J. 385: 399-408. 2005), which is a cell-permeable quinoxaline compound and is a low-molecular-weight compound specifically inhibiting the activity of Akt1, Akt2, and Akt3; GSK 690693 (Rhodes et al., Cancer Res. 68 (7): 2366-2374. 2008), which is a compound derived from Aminofurazan and is a low-molecular-weight compound specifically inhibiting the activity of Akt1, Akt2, and Akt3; AT7867 (Grimshaw et al., Mol. Cancer Ther. 9 (5): 1100-10. 2010), which is a low-molecular-weight compound specifically inhibiting the activity of Akt1, Akt2, and Akt3; and RNAi molecules against DNA encoding AKT3, ribozymes, antisense nucleic acids, DNA/RNA chimeric polynucleotides, and vectors for expressing them, anti-AKT3 antibodies, and dominant negative variants of AKT3. These drugs are commercially available or can be appropriately produced on the basis of publicly known techniques.

Examples of the drug inhibiting the activity of EIF4B among the PI3K signaling pathway-related proteins that exhibit synthetic lethality when inhibited together with GST-π include, but are not limited to, RNAi molecules against DNA encoding EIF4B, ribozymes, antisense nucleic acids, DNA/RNA chimeric polynucleotides, and vectors for expressing them, anti-EIF4B antibodies, and dominant negative variants of EIF4B. These drugs are commercially available or can be appropriately produced on the basis of publicly known techniques.

Examples of the drug inhibiting the activity of EIF4E among the PI3K signaling pathway-related proteins that exhibit synthetic lethality when inhibited together with GST-πinclude, but are not limited to, 4EGI-1 (Moerke et al., Cell. 128: 257-267. 2007), which is a low-molecular-weight compound specifically inhibiting EIF4E/EIF4G interaction; and RNAi molecules against DNA encoding EIF4E, ribozymes, antisense nucleic acids, DNA/RNA chimeric polynucleotides, and vectors for expressing them, anti-EIF4E antibodies, and dominant negative variants of EIF4E. These drugs are commercially available or can be appropriately produced on the basis of publicly known techniques.

Examples of the drug inhibiting the activity of ILK among the PI3K signaling pathway-related proteins that exhibit synthetic lethality when inhibited together with GST-π include, but are not limited to, Cpd 22 (Lee et al., J. Med. Chem. 54, 6364. 2011), which is a cell-permeable pyrazole compound and is a low-molecular-weight compound specifically inhibiting ILK; QLT0267 (Younes et al., Mol Cancer Ther. August; 4 (8): 1146-56. 2005), which is a low-molecular-weight compound specifically inhibiting the kinase activity of ILK; and RNAi molecules against DNA encoding ILK, ribozymes, antisense nucleic acids, DNA/RNA chimeric polynucleotides, and vectors for expressing them, anti-ILK antibodies, and dominant negative variants of ILK. These drugs are commercially available or can be appropriately produced on the basis of publicly known techniques.

Examples of the drug inhibiting the activity of MTCP1 among the PI3K signaling pathway-related proteins that exhibit synthetic lethality when inhibited together with GST-π include, but are not limited to, RNAi molecules against DNA encoding MTCP1, ribozymes, antisense nucleic acids, DNA/RNA chimeric polynucleotides, and vectors for expressing them, anti-MTCP1 antibodies, and dominant negative variants of MTCP1. These drugs are commercially available or can be appropriately produced on the basis of publicly known techniques.

Examples of the drug inhibiting the activity of PIK3CA among the PI3K signaling pathway-related proteins that exhibit synthetic lethality when inhibited together with GST-π include, but are not limited to, HS-173 (Lee et al., Cancer Lett. January 1; 328 (1): 152-9. 2013), which is a low-molecular-weight compound specifically inhibiting PI3K; CUDC-907 (Qian et al., Clin Cancer Res. 18 (15): 4104-4113. 2012), which is a low-molecular-weight compound specifically inhibiting the activity of both HDAC and PI3K; PKI-402 (Dehnhardt et al., J Med Chem. January 28; 53 (2): 798-810. 2010), which is a low-molecular-weight compound specifically inhibiting the activity of both PI3K and mTOR; PF-04691502 (Yuan et al., Mol Cancer Ther, 10 (11), 2189-2199. 2011), which is a low-molecular-weight compound specifically inhibiting the activity of both PI3K and mTOR; NVP-BGT226 (Glienke et al., Tumor Biology, 33 (3): 757-765. 2012), which is a low-molecular-weight compound specifically inhibiting the activity of both PI3K and mTOR; and BYL719 (Furet et al., Bioorg Med Chem Lett 2013; 23: 3741-3748. 2013), SAR245409 (XL765) (Dai et.al, Endocrinology. March; 154 (3):1247-592013), ZSTK474 (Toyama et.al, Arthritis Res Ther. 12 (3): R92. 2010), VS-5584 (SB2343) (Hart et.al, Mol Cancer Ther. 2013 February; 12 (2):151-61. 2013), PIK-75 (Zheng et.al, Mol Pharmacol. October; 80 (4):657-64. 2011), PIK-90 (Van et.al, J Cell Biol. 2006 Jul. 31; 174 (3):437-45. 2006), A66 (Jamieson et.al, Biochem J. 438:53-62. 2011), CNX1351 (Nacht et.al, J Med Chem, 56 (3): 712-721. 2013), PF-4989216 (Walls et.al, Clin Cancer Res. 2014 Feb. 1; 20 (3):631-43. 2014), BKM120 (Bendell et.al, J. Clin. Oncol. 30 (3): 282-90. 2012), BEZ235 Tosylate (Maira et.al, Mol Cancer Ther 2008; 7: 1851-1863. 2008), LY294002 (Maira et.al, Biochem. Soc. Trans. 37 (PO): 265-72. 2009), PI-103 (Raynaud et.al, Molecular Cancer Therapeutics, 8 (7): 1725-1738. 2009), XL147 (Shapiro et.al, Proc 97th Annu Meet AACR, 14-18. 2007), CH5132799 (Ohwada et.al, Bioorganic & medicinal chemistry letters, 21 (6): 1767-1772. 2011), BAY 80-6946 (Copanlisib) (Patnaik et.al, J Clin Oncol, 29, 2011), GDC-0032 (Ndubaku et.al, J Med Chem. June 13; 56 (11):4597-610. 2013), XL765 (Laird et.al, AACR-NCI-EORTC International Conference on Molecular Targets and Cancer Therapeutics. October p.B250 2007), and RNAi molecules against DNA encoding PIK3CA, ribozymes, antisense nucleic acids, DNA/RNA chimeric polynucleotides, and vectors for expressing them, anti-PIK3CA antibodies, and dominant negative variants of PIK3CA. These drugs are commercially available or can be appropriately produced on the basis of publicly known techniques.

Examples of the drug inhibiting the activity of SRF among the PI3K signaling pathway-related proteins that exhibit synthetic lethality when inhibited together with GST-πinclude, but are not limited to, CCG-1423 (Evelyn et al., Mol. Cancer Ther. 6, 2249. 2007), which is a cell-permeable benzamide compound and is a low-molecular-weight compound specifically inhibiting the RHO signaling pathway and SRF activation; CCG-100602 (Evelyn, et al., Bioorg Med Chem Lett 20 665-72. 2010), which is a CCG-1423 analog and is a low-molecular-weight compound specifically inhibiting the RHO signaling pathway and SRF activation; and RNAi molecules against DNA encoding SRF, ribozymes, antisense nucleic acids, DNA/RNA chimeric polynucleotides, and vectors for expressing them, anti-SRF antibodies, and dominant negative variants of SRF. These drugs are commercially available or can be appropriately produced on the basis of publicly known techniques.

Particularly, RNAi molecules against DNA encoding the protein, ribozymes, antisense nucleic acids, DNA/RNA chimeric polynucleotides, and vectors for expressing them are preferable as the drug inhibiting the production or activity of the cell cycle-regulating protein (e.g., p21) or anti-apoptosis-related protein or PI3K signaling pathway-related protein that exhibits synthetic lethality when inhibited together with GST-π, because of high specificity and a low possibility of adverse reactions.

The inhibition of the homeostasis-related protein can be determined when the expression or activity of the protein in the cell is inhibited as compared with the case the agent inhibiting the protein is not allowed to act thereon. The expression of the protein can be evaluated without limitations by a known arbitrary approach, for example, an immunoprecipitation method using an antibody, EIA, ELISA, IRA, IRMA, Western blotting, an immunohistochemical method, an immunocytochemical method, flow cytometry, or various hybridization methods, Northern blotting, Southern blotting, or various PCR methods which employ nucleic acids specifically hybridizing to a nucleic acid encoding the protein or a unique fragment thereof, or a transcript (e.g., mRNA) or a splicing product of the nucleic acid.

Also, the activity of, for example, p21 can be evaluated without limitations by analyzing the known activity of p21, for example, binding activity against cyclin-CDK2 or a cyclin-CDK1 complex by a known arbitrary method, for example, an immunoprecipitation method, Western blotting, mass spectrometry, a pull-down method, or a surface plasmon resonance (SPR) method.

As used herein, the RNAi molecule refers to an arbitrary molecule that brings about RNA interference. The RNAi molecule is not limited and includes double-stranded RNAs such as siRNA (small interfering RNA), miRNA (micro RNA), shRNA (short hairpin RNA), ddRNA (DNA-directed RNA), piRNA (Piwi-interacting RNA), rasiRNA (repeat associated siRNA), and alternatives thereof, and the like. These RNAi molecules are commercially available or may be designed and prepared on the basis of publicly known sequence information, i.e., the nucleotide sequences and/or amino acid sequences shown in SEQ ID NOs: 1 to 30, 39 to 108.

As used herein, the antisense nucleic acid includes RNA, DNA, PNA, or complexes thereof.

As used herein, the DNA/RNA chimeric polynucleotide is not limited and includes, for example, a double-stranded polynucleotide described in JP Patent Publication (Kokai) No. 2003-219893 A (2003) which consists of DNA and RNA and inhibits the expression of a target gene.

The drug inhibiting GST-π and the drug inhibiting a homeostasis-related protein that exhibits synthetic lethality when inhibited together with GST-π may be contained in a single preparation or may be separately contained in two or more preparations. In the latter case, these preparations may be administered at the same time or may be administered in a staggered manner. In the case of administration in a staggered manner, the preparation containing the drug inhibiting GST-π may be administered before or after the administration of the preparation containing the drug inhibiting a homeostasis-related protein that exhibits synthetic lethality when inhibited together with GST-π.

The cell death-inducing agent and the cell growth-inhibiting agent according to the present invention may comprise one type of the aforementioned homeostasis-related protein, or two or more types the aforementioned homeostasis-related protein. For example, two or more types of cell cycle-regulating protein; two or more types of anti-apoptosis-related proteins; or one or more types of cell cycle-regulating proteins and one or more types of anti-apoptosis-related proteins may be used as homeostasis-related proteins included in the cell death-inducing agent and the cell growth-inhibiting agent according to the present invention.

Incidentally, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF are each a homeostasis-related protein that exhibits synthetic lethality for a cancer cell when inhibited together with GST-π. Accordingly, the drug inhibiting the protein serves as an active ingredient for an agent or a composition which potentiates the induction of cell death and/or the inhibition of cell growth by the drug inhibiting GST-π (hereinafter, also referred to as a “cell death induction-potentiating agent”, a “cell growth inhibition-potentiating agent”, a “composition for the potentiation of cell death induction”, or a “composition for the potentiation of cell growth inhibition”). In other words, the induction of cell death and/or the inhibition of cell growth by the administration of the drug inhibiting GST-π can be potentiated by administering an effective amount of the drug inhibiting the protein.

The content of the active ingredient in the agent or the composition of the present invention may be an amount that induces cell death such as apoptosis and/or inhibits cell growth when the agent or the composition is administered. Also, an amount that does not have adverse effect exceeding advantages brought about by administration is preferable. Such an amount is publicly known or can be appropriately determined by an in vitro test using cultured cells or the like or by a test in model animals such as mice, rats, dogs, or pigs. Such a testing method is well known to those skilled in the art. The induction of apoptosis can be evaluated by various known approaches, for example, the detection of apoptosis-specific phenomena such as DNA fragmentation, binding of annexin V to a cell membrane, change in mitochondrial membrane potential, and activation of caspase, and TUNEL staining. Also, the inhibition of cell growth can be evaluated by various known approaches, for example, the time-dependent measurement of the number of live cells, the measurement of the size, volume, or weight of tumor, the measurement of the amount of DNA synthesized, a WST-1 method, a BrdU (bromodeoxyuridine) method, and a 3H thymidine incorporation method. The content of the active ingredient can vary depending on the dosage form of the agent or the composition. For example, in the case of using a plurality of units of compositions in one administration, the amount of the active ingredient contained in one composition unit can be set to 1/a plurality of amounts of the active ingredient necessary for one administration. Such adjustment of the content can be appropriately carried out by those skilled in the art.

Moreover, the drug inhibiting GST-π and the drug inhibiting a homeostasis-related protein that exhibits synthetic lethality when inhibited together with GST-π can be formulated as active ingredients to thereby produce a cell death-inducing agent, a cell growth-inhibiting agent, a composition for cell death induction, or a composition for cell growth inhibition.

Furthermore, a combination of the drug inhibiting GST-π and the drug inhibiting a homeostasis-related protein that exhibits synthetic lethality when inhibited together with GST-π for use in cell death induction or cell growth inhibition can be provided. In addition, a method for inducing cell death or a method for inhibiting cell growth, comprising administering effective amounts of the drug inhibiting GST-π and the drug inhibiting a homeostasis-related protein that exhibits synthetic lethality when inhibited together with GST-π can be provided.

All of the aforementioned methods for inducing cell death such as apoptosis or inhibiting cell growth may be in vitro methods or may be in vivo methods. The drugs for these methods are as already mentioned above, and the effective amount of each drug may be an amount that induces cell death such as apoptosis and/or inhibits cell growth in the recipient cell. Also, an amount that does not have adverse effect exceeding advantages brought about by administration is preferable. Such an amount is publicly known or can be appropriately determined by an in vitro test using cultured cells. Such a testing method is well known to those skilled in the art. The induction of cell death or the inhibition of cell growth can be evaluated by various known approaches including those mentioned above. When the drug is administered to a predetermined cancer cell population, the effective amount does not have to bring about cell death or growth inhibition for all cells in this cell population. The effective amount may be, for example, an amount that brings about apoptosis or growth inhibition for 1% or more, 2% or more, 3% or more, 4% or more, 5% or more, 6% or more, 8% or more, 10% or more, 12% or more, 15% or more, 20% or more, and 25% or more of the cells in the cell population.

The cell death-inducing agent or the cell growth-inhibiting agent of the present invention can effectively induce cell death or inhibit cell growth even for a cancer cell and as such, is effective as an ingredient for a pharmaceutical composition for a disease caused by abnormal cell growth. Also, the drug inhibiting GST-π and the drug inhibiting a homeostasis-related protein that exhibits synthetic lethality when inhibited together with GST-π can be formulated as active ingredients to thereby produce a pharmaceutical composition for a disease caused by abnormal cell growth. Furthermore, the treatment or therapy of a disease caused by abnormal cell growth, comprising administering an effective amount of the produced pharmaceutical composition to a subject in need thereof can be provided.

The pharmaceutical composition is effective for treating a disease caused by abnormal cell growth, particularly, for treating a disease having cell death or abnormal cell growth by expressing mutated KRAS.

The disease caused by a cell expressing mutated KRAS is not limited and includes, for example, benign or malignant tumors (also referred to as cancers or malignant neoplasms), hyperplasia, keloid, Cushing syndrome, primary aldosteronism, erythroplakia, polycythemia vera, leukoplakia, hyperplastic scar, lichen planus, and lentiginosis.

Examples of the cancer according to the present invention include cancers, cancers highly expressing GST-π, and cancers caused by cells expressing mutated KRAS (also simply referred to as KRAS cancers). In many cases, the KRAS cancers are included in the cancers highly expressing GST-π. Examples thereof include, but are not limited to: sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma; carcinomas such as brain tumor, head and neck cancer, breast cancer, lung cancer, esophageal cancer, stomach cancer, duodenal cancer, appendix cancer colorectal cancer, rectal cancer, liver cancer, pancreatic cancer, gallbladder cancer, bile duct cancer, anus cancer, kidney cancer, urethral cancer, urinary bladder cancer, prostate cancer, penis cancer, testis cancer, uterine cancer, ovary cancer, vulval cancer, vaginal cancer, and skin cancer; and leukemia and malignant lymphoma. In the present invention, the “cancer” includes epithelial malignant tumors and non-epithelial malignant tumors. The cancer according to the present invention may be present in an arbitrary site of the body, for example, the brain, the head and neck region, the chest, the extremities, the lung, the heart, thymus glands, the esophagus, the stomach, the small intestine (duodenum, jejunum, and ileum), the large intestine (colon, cecum, appendix, and rectum), the liver, the pancreas, the gallbladder, the anus, the kidney, urinary ducts, the urinary bladder, the prostate, the penis, the testis, the uterus, the ovary, the vulva, the vagina, the skin, striated muscles, smooth muscles, synovial membranes, cartilage, bone, thyroid glands, adrenal glands, the peritoneum, the mesenterium, bone marrow, blood, the vascular system, the lymphatic system such as lymph nodes, or lymph.

For the pharmaceutical composition, the drug inhibiting GST-π and the drug inhibiting a homeostasis-related protein that exhibits synthetic lethality when inhibited together with GST-π may be used in combination with an additional active ingredient. In this context, the use in combination includes, for example, the administration of the additional active ingredient as another preparation, and the administration of the additional active ingredient as a combination drug with at least one of the other drugs. In the case of administration as another preparation, the preparation containing the additional active ingredient may be administered before, at the same time with, or after the administration of the other preparation(s).

Examples of such an additional active ingredient include ones effective for the treatment of the disease of interest. For example, when the disease to be treated is a cancer, an anticancer agent can be used in combination therewith. Examples of the anticancer agent can include: alkylating agents such as ifosfamide, nimustine hydrochloride, cyclophosphamide, dacarbazine, melphalan, and ranimustine; metabolic antagonists such as gemcitabine hydrochloride, enocitabine, cytarabine ocfosfate, cytarabine preparations, tegafur uracil, tegafur-gimeracil-oteracil potassium combination drugs (e.g., TS-1), doxifluridine, hydroxycarbamide, fluorouracil, methotrexate, and mercaptopurine; antitumor antibiotics such as idarubicin hydrochloride, epirubicin hydrochloride, daunorubicin hydrochloride, daunorubicin citrate, doxorubicin hydrochloride, pirarubicin hydrochloride, bleomycin hydrochloride, peplomycin sulfate, mitoxantrone hydrochloride, and mitomycin C; alkaloids such as etoposide, irinotecan hydrochloride, vinorelbine tartrate, docetaxel hydrate, paclitaxel, vincristine sulfate, vindesine sulfate, and vinblastine sulfate; hormone therapy agents such as anastrozole, tamoxifen citrate, toremifene citrate, bicalutamide, flutamide, and estramustine phosphate; platinum complexes such as carboplatin, cisplatin (CDDP), and nedaplatin; anti-angiogenic agents such as thalidomide, Neovastat, and bevacizumab; and L-asparaginase.

When the active ingredients in various agents or compositions, the method of treating a subject, etc., of the present invention described herein are nucleic acids, for example, RNAi molecules, ribozymes, antisense nucleic acids, or DNA/RNA chimeric polynucleotides, they may be used directly as naked nucleic acids or may be supported by various vectors. Publicly known arbitrary vectors such as plasmid vectors, phage vectors, phagemid vectors, cosmid vectors, or virus vectors can be used as the vectors. It is preferable that the vectors should contain at least a promoter that potentiates the expression of each nucleic acid to be carried. In this case, it is preferable that the nucleic acid should be operably linked to such a promoter. The nucleic acid operably linked to the promoter means that the nucleic acid and the promoter are located such that the protein encoded by the nucleic acid is properly produced by the action of the promoter. The vectors may or may not be replicable in host cells. Also, the transcription of the gene may be performed outside the nuclei of the host cells or may be performed inside the nuclei thereof. In the latter case, the nucleic acid may be integrated into the genomes of the host cells.

Alternatively, the active ingredients may be supported by various non-viral lipid or protein carriers. Examples of such carriers include, but are not limited to, cholesterols, liposomes, antibody protomers, cyclodextrin nanoparticles, fusion peptides, aptamers, biodegradable polylactic acid copolymers, and polymers, which can enhance the efficiency of cellular uptake (see e.g., Pirollo and Chang, Cancer Res. 2008; 68 (5): 1247-50). Particularly, cationic liposomes or polymers (e.g., polyethyleneimine) are useful. Further examples of the polymers useful as such carriers include those described in, for example, US 2008/0207553 and US 2008/0312174.

In various pharmaceutical compositions of the present invention described herein, the active ingredients may be combined with an additional arbitrary ingredient unless the effects of the active ingredients are impaired. Examples of such an arbitrary ingredient include other chemotherapeutic agents, pharmacologically acceptable carriers, excipients, and diluents. Furthermore, the compositions may be coated with suitable materials, for example, enteric coatings or time-controlled disintegrating materials, according to administration routes, drug release manners, etc., and may be incorporated into suitable drug release systems.

Various agents and compositions (including various pharmaceutical compositions) of the present invention described herein may be administered without limitations through various routes including both oral and parenteral routes, for example, oral, intravenous, intramuscular, subcutaneous, local, intratumoral, rectal, intraarterial, intraportal, intraventricular, transmucosal, percutaneous, nasal, intraperitoneal, intrapulmonary, and intrauterine routes, or may be prepared into dosage forms suitable for each administration route. Arbitrary publicly known ones can be appropriately adopted for such dosage forms and preparation methods (see e.g., Hyoujun Yakuzai Gaku (Standard Pharmaceutics in English), edited by Yoshiteru Watanabe et al., Nankodo Co., Ltd., 2003).

Examples of the dosage form suitable for oral administration include, but are not limited to, powders, granules, tablets, capsules, solutions, suspensions, emulsions, gels, and syrups. Also, examples of the dosage form suitable for parenteral administration include injections such as injections in a solution state, injections in a suspension state, injections in an emulsion state, and injections of type to be prepared before use. The preparations for parenteral administration can be in the form of an aqueous or nonaqueous isotonic sterile solution or suspension.

Various agents or compositions (including various pharmaceutical compositions) of the present invention described herein may be prepared to target particular tissues or cells. The targeting can be achieved by a known arbitrary approach. In the case of intending delivery to a cancer, for example, an approach such as passive targeting by setting the size of a preparation to a diameter of 50 to 200 μm, particularly, 75 to 150 μm, suitable for the exertion of EPR (enhanced permeability and retention) effects, or active targeting using a ligand such as CD19, HER2, transferrin receptor, folate receptor, VIP receptor, EGFR (Torchilin, AAPS J. 2007; 9 (2): E128-47), RAAG10 (JP Patent Publication (Kohyo) No. 2005-532050 A (2005)), PIPA (JP Patent Publication (Kohyo) No. 2006-506071 A (2006)), or KID3 (JP Patent Publication (Kohyo) No. 2007-529197 A (2007)), a peptide having an RGD motif or an NGR motif, F3, LyP-1 (Ruoslahti et al., J Cell Biol. 2010; 188 (6): 759-68), or the like as a targeting agent can be used without limitations. Since it is also known that retinoid or a derivative thereof is useful as a targeting agent for a cancer cell (WO 2008/120815), a carrier comprising retinoid as a targeting agent may be used. Such a carrier is described in WO 2009/036368, WO 2010/014117, WO 2012/170952, etc., in addition to the above literature.

Various agents or compositions (including various pharmaceutical compositions) of the present invention described herein can be supplied in any form and may be provided in a form that can be prepared before use, for example, a form that can be prepared by a physician and/or a pharmacist, a nurse, or other paramedical persons in a medical setting or in the neighborhood thereof, from the viewpoint of preservation stability. Such a form is particularly useful when the agent or the composition of the present invention comprises ingredients difficult to stably preserve, such as lipids, proteins, or nucleic acids. In this case, the agent or the composition of the present invention is provided as one or two or more containers comprising at least one of the components essential therefor, and is prepared before use, for example, within 24 hours before use, preferably within 3 hours before use, more preferably immediately before use. For the preparation, a reagent, a solvent, a prescription instrument, or the like usually available in the preparation location can be appropriately used.

Thus, the present invention also relates to a composition preparation kit comprising one or two or more containers comprising one or a combination of the active ingredients that may be contained in various agents or compositions of the present invention, and necessary components for various agents or compositions which are provided in the form of such a kit. The kit of the present invention may additionally comprise an instruction, for example, a manual or an electronic recording medium (CD or DVD), describing preparation methods, a method of treating a subject, etc., for various agents or compositions of the present invention. Also, the kit of the present invention may comprise all of the components for completing various agents or compositions of the present invention, but is not necessarily required to comprise all of the components. Thus, the kit of the present invention may not comprise a reagent or a solvent usually available in a medical setting, an experimental facility, or the like, for example, sterile water, saline, or a glucose solution.

The effective amount for various methods of treating a subject of the present invention described herein is, for example, an amount that reduces the symptoms of a disease or delays or terminates the progression of the disease, and is preferably an amount that inhibits or cures the disease. Also, an amount that does not have adverse effect exceeding advantages brought about by administration is preferable. Such an amount can be appropriately determined by an in vitro test using cultured cells or the like or by a test in model animals such as mice, rats, dogs, or pigs. Such a testing method is well known to those skilled in the art. Furthermore, the doses of the drugs used in the treatment method of the present invention are generally known to those skilled in the art or can be appropriately determined by the aforementioned tests or the like.

The specific doses of the active ingredients to be administered in the method of treating a subject of the present invention described herein can be determined in consideration of various conditions about a subject in need of treatment, for example, the severity of the symptoms, the general health state of the subject, the age, the body weight, the sex of the subject, diet, the time and frequency of administration, a drug used in combination, response to the therapy, dosage form, and compliance to the therapy.

The administration route includes various routes including both oral and parenteral routes, for example, oral, intravenous, intramuscular, subcutaneous, local, intratumoral, rectal, intraarterial, intraportal, intraventricular, transmucosal, percutaneous, nasal, intraperitoneal, intrapulmonary, and intrauterine routes.

The frequency of administration differs depending on the properties of the agent or the composition used, and conditions about the subject including those described above, and may be, for example, multiple times per day (i.e., 2, 3, 4, or 5 times a day), once a day, every few days (i.e., every 2, 3, 4, 5, 6, or 7 days), once a week, or every few weeks (i.e., every 2, 3, or 4 weeks).

As used herein, the term “subject” means an arbitrary organism individual and is preferably an animal, more preferably a mammal, further preferably a human individual. In the present invention, the subject may be healthy or may have some disease. In the case of intending the treatment of a particular disease, the subject typically means a subject having this disease or having the risk of being affected by this disease.

As used herein, the term “treatment” includes every type of medically acceptable prophylactic and/or therapeutic intervention aimed at, for example, curing, temporarily ameliorating, or preventing a disease. For example, the term “treatment” encompasses various medically acceptable interventions of interest, including the delay or termination of the progression of a disease, the involution or disappearance of a lesion, the prevention of onset or the prevention of recurrence, etc.

Incidentally, as mentioned above, ATM, CDC25A, p21, PRKDC, RBBP8, SKP2, MCM10, RNPC1, CCNL1, CENPH, BRSK1, MYLK, MCM8, CCNB3, MCMDC1, AATF, AKT1, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, MYO18A, MTOR, IRAK1, IRS1, MYD88, NFKB1, PIK3CG, RAC1, AKT3, EIF4B, EIF4E, ILK, MTCP1, PIK3CA and SRF are each a protein that exhibits synthetic lethality for a cancer cell when inhibited together with GST-π. Thus, a cell death-inducing agent and/or a cell growth-inhibiting agent for a cancer cell that is used together with a drug inhibiting GST-π can be screened for by using the inhibition of this homeostasis-related protein as an index. Specifically, a substance that can inhibit the homeostasis-related protein serves as a candidate substance for the cell death-inducing agent and/or the cell growth-inhibiting agent for a cancer cell that is used together with a drug inhibiting GST-π.

For example, a test substance is contacted with a cell expressing mutated KRAS as one example of the cancer cell, and the expression level of the homeostasis-related protein that exhibits synthetic lethality for the cell expressing mutated KRAS when inhibited together with GST-π is measured in the cell. The test substance can be selected as a candidate substance for a drug inhibiting the homeostasis-related protein, when the expression level measured after the contact of the test substance is decreased compared with the expression level measured in the absence of the test substance.

On the other hand, the drug inhibiting GST-π is a protein that exhibits synthetic lethality for a cancer cell when inhibited together with the drug inhibiting the homeostasis-related protein that exhibits synthetic lethality for a cancer cell when inhibited together with GST-π. Thus, a cell death-inducing agent and/or a cell growth-inhibiting agent for a cancer cell that is used together with a drug inhibiting the homeostasis-related protein can be screened for by using the inhibition of GST-π as an index. Specifically, a substance that can inhibit GST-π serves as a candidate substance for the cell death-inducing agent and/or the cell growth-inhibiting agent for a cancer cell that is used together with a drug inhibiting the homeostasis-related protein.

For example, a test substance is contacted with a cell expressing mutated KRAS as one example of the cancer cell, and the expression level of GST-π is measured in the cell. The test substance can be selected as a candidate substance for a drug inhibiting GST-π, when the expression level measured after the contact of the test substance is decreased compared with the expression level measured in the absence of the test substance.

Likewise, a cell death-inducing agent and/or a cell growth-inhibiting agent for a cancer cell can be screened for by using both of the inhibition of GST-π and the inhibition of the homeostasis-related protein that exhibits synthetic lethality for a cancer cell when inhibited together with GST-π as indexes. Specifically, a substance that can inhibit GST-π and can inhibit the homeostasis-related protein serves as a candidate substance for the cell death-inducing agent and/or the cell growth-inhibiting agent for a cancer cell.

For example, a test substance is contacted with a cell expressing mutated KRAS as one example of the cancer cell, and the expression level of GST-π and the expression level of the homeostasis-related protein are measured in the cell. The test substance can be selected as a candidate substance for a drug inhibiting GST-π and inhibiting the homeostasis-related protein that exhibits synthetic lethality for a cancer cell when inhibited together with GST-π, when these expression levels measured after the contact of the test substance are both decreased compared with the respective expression levels measured in the absence of the test substance.

In this context, the test substance is not limited by any means and can be any substance. The test substance may be a single substance or may be a mixture consisting of a plurality of components. The test substance may be configured to comprise an unidentified substance as in, for example, an extract from a microorganism or a culture solution, or may be configured to comprise known compositions at predetermined compositional ratios. Also, the test substance may be any of proteins, nucleic acids, lipids, polysaccharides, organic compounds, and inorganic compounds.

### EXAMPLES

Hereinafter, the present invention will be described further specifically with reference to Examples. However, the technical scope of the present invention is not intended to be limited by Examples below.

[Experiment 1] Knockdown of GST-π and P21 by siRNAs

As examples of cancer cells, 1×10⁵ M7609 cells (human colorectal cancer cells having KRAS mutation) and PANC-1 cells (human pancreatic cancer cells having KRAS mutation) were inoculated to 6 cm Petri dishes and cultured for 18 hours in Roswell Park Memorial Institute 1640 (RPMI 1640, Sigma-Aldrich Corp.) supplemented with 10% fetal bovine serum (FBS) and 0.5% L-glutamine. The culture conditions were 37° C. and 5% CO₂, unless otherwise specified. Moreover, as an example of cancer cells, 0.5×10⁵ A549 cells (human lung cancer cells having KRAS mutation) were inoculated to a 6 cm Petri dish and cultured for 18 hours in a Dulbecco's modified Eagle's medium (DMEM, Sigma-Aldrich Corp.) supplemented with 10% FBS and 1% L-glutamine. Furthermore, as an example of cancer cells, 1×10⁵ MIA PaCa-2 cells (human pancreatic cancer cell having KRAS mutation) were inoculated to a 6 cm Petri dish and cultured for 18 hours in DMEM supplemented with 10% FBS and 1% L-glutamine. Furthermore, as an example of cancer cells, 0.5×10⁵ HCT116 cells (human colorectal cancer cells having KRAS mutation) were inoculated to a 6 cm Petri dish and cultured for 18 hours in McCoy's 5A Medium (McCoy, Sigma-Aldrich Corp.) supplemented with 10% FBS and 0.5% L-glutamine.

In this experiment, first, the PANC-1, A549, or MIA PaCa-2 cells that became 20 to 30% confluent were transfected with GST-π siRNA and/or P21 siRNA using Lipofectamine RNAi MAX (Life Technologies Corp.) as follows.

The Lipofectamine/siRNA mixed solution for transfection was prepared as follows: first, a Lipofectamine solution in which 15 μL of Lipofectamine RNAi MAX and 485 μL of OPTI-MEM (Sigma-Aldrich Corp.) were mixed was prepared. Next, an siRNA solution in which a predetermined amount of 50 μM siRNA was adjusted to 500 μL with OPTI-MEM was prepared (e.g., in the case of preparing an siRNA solution used with a final concentration of 50 nM, 6 μL of 50 μM siRNA and 494 μL of OPTI-MEM were mixed), and this was mixed with the aforementioned Lipofectamine solution and left standing at room temperature for 15 minutes. siRNAs given below were used. In the description below, the upper-case letters represent RNAs, and the lower-case letters represent DNAs.

GST-π siRNA:
Sense strand:
(SEQ ID NO: 31)
GGGAGGCAAGACCUUCAUUtt
Antisense strand:
(SEQ ID NO: 32)
AAUGAAGGUCUUGCCUCCCtg
P21 siRNA:
Sense strand:
(SEQ ID NO: 33)
UCCUAAGAGUGCUGGGCAUtt
Antisense strand:
(SEQ ID NO: 34)
AUGCCCAGCACUCUUAGGAtt
Control siRNA:
Sense strand:
(SEQ ID NO: 35)
ACGUGACACGUUCGGAGAAtt
Antisense strand:
(SEQ ID NO: 36)
UUCUCCGAACGUGUCACGUtt
GST-π siRNA-2:
Sense strand:
(SEQ ID NO: 37)
UCUCCCUCAUCUACACCAAtt
Antisense strand:
(SEQ ID NO: 38)
UUGGUGUAGAUGAGGGAGAtg

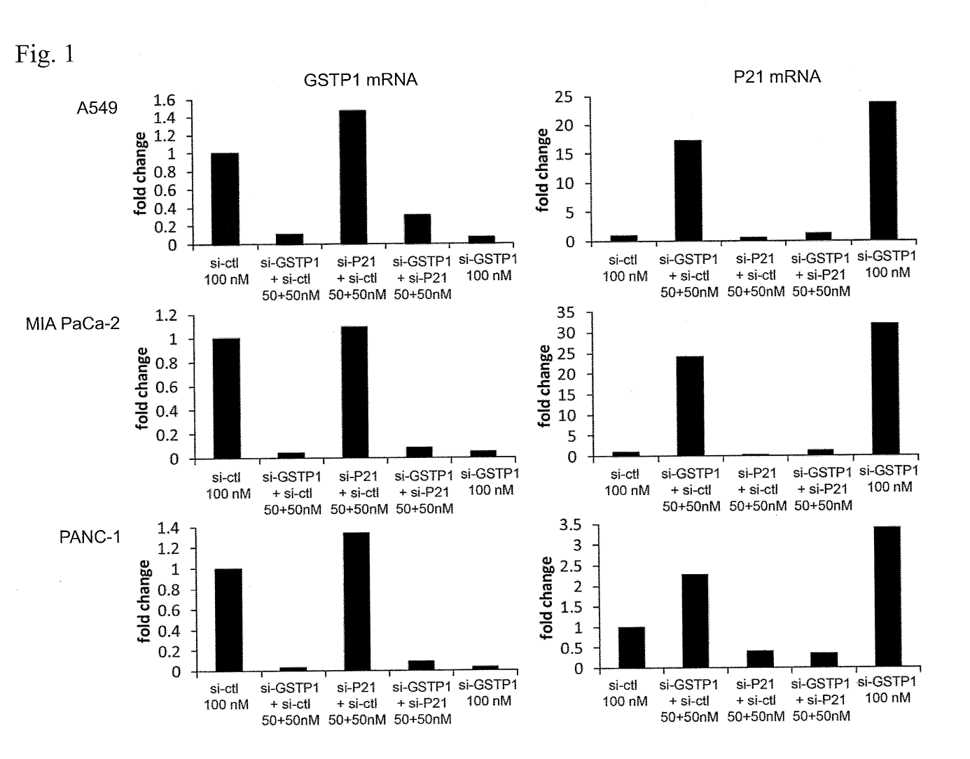

GST-π siRNA and P21 siRNA each at a final concentration of 50 nM, GST-π siRNA or P21 siRNA at a final concentration of 50 nM (both added with control siRNA at a final concentration of 50 nM), or GST-π siRNA at a final concentration of 100 nM (without the addition of control siRNA) were added to each of the Petri dishes containing the PANC-1, MIA PaCa-2, or A549 cells. For a control used, control siRNA was added at a final concentration of 100 nM. After culture for 1 day without the replacement of the medium, the GST-π mRNA level and the P21 mRNA level were quantified by quantitative PCR using 7300 Real Time PCR System (Applied Biosystems, Inc.). The results are shown in FIG. 1. As shown in FIG. 1, it was revealed that the amount of P21 mRNA is increased by knocking down GST-π by the siRNA.

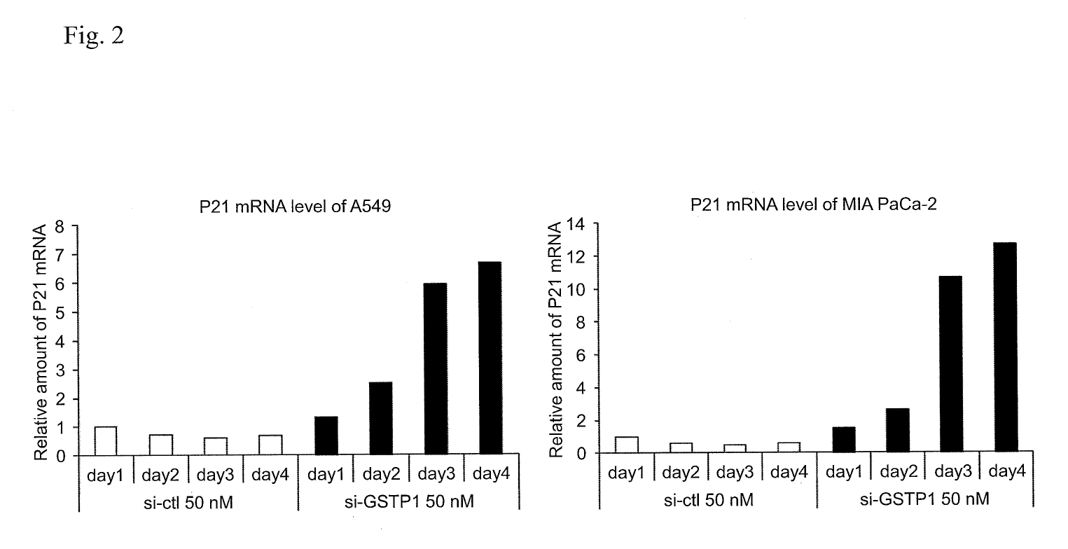

Moreover, as for the case where GST-π siRNA or control siRNA at a final concentration of 50 nM was added to each of the Petri dishes containing the A549 cells or the MIA PaCa-2 cells, the P21 mRNA level was similarly quantified every day from the day of addition of GST-π siRNA or control siRNA to the 4th day. The results are shown in FIG. 2. As shown in FIG. 2, it was revealed that the expression level of P21 mRNA is increased over time by knocking down GST-π by the siRNA.

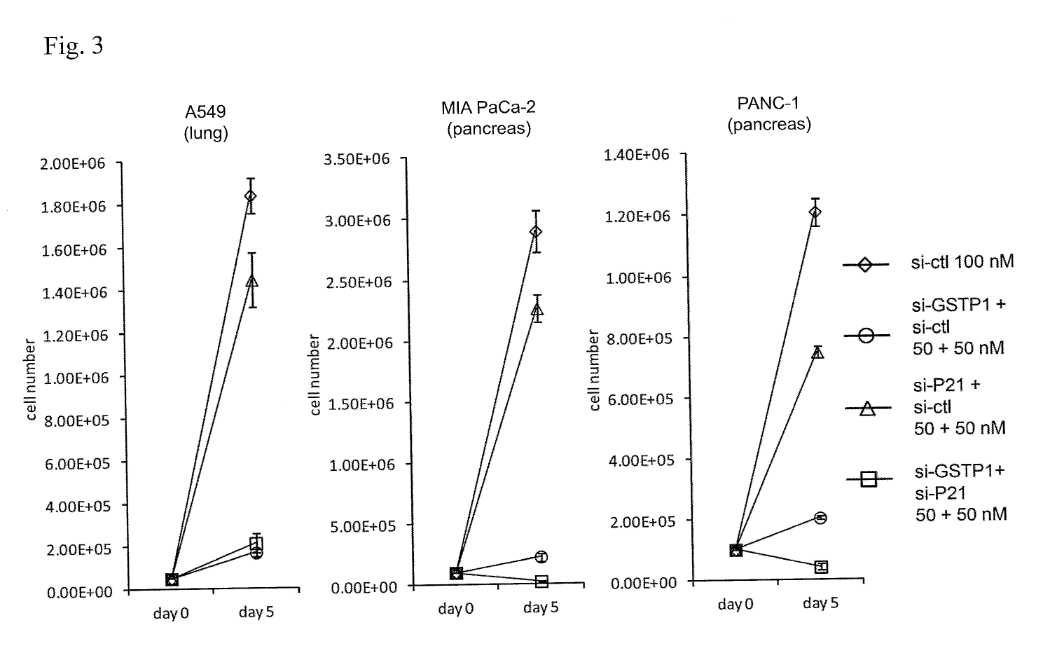

Meanwhile, the influence of GST-π siRNA and/or P21 siRNA on the number of cells was tested. First, GST-π siRNA and P21 siRNA each at a final concentration of 50 nM, or GST-π siRNA or P21 siRNA at a final concentration of 50 nM (both added with control siRNA at a final concentration of 50 nM) were added to each of the Petri dishes containing the PANC-1, MIA PaCa-2, or A549 cells. For a control used, control siRNA was added at a final concentration of 100 nM. After culture for 5 days without the replacement of the medium, the cells were dissociated and collected from the Petri dish by trypsin treatment, and the number of cells was counted. The results are shown in FIG. 3. As shown in FIG. 3, it is evident that when GST-π and P21 are knocked down each alone using GST-π siRNA or P21 siRNA, the number of cells cannot be decreased with respect to the number of inoculated cells, though cell growth is inhibited. However, it is evident that when GST-π and P21 are both knocked down using GST-π siRNA and P21 siRNA, not only is growth inhibited but cell death can be induced in the PANC-1 cells and the MIA PaCa-2 cells expressing mutated KRAS.

From FIG. 3, it was considered, as to the A549 cells expressing mutated KRAS, that cell death was not induced by the treatment mentioned above. Accordingly, the number of transfections with GST-π siRNA and P21 siRNA was increased, and the influence of GST-π siRNA and/or P21 siRNA on the number of cells was tested as to the A549 cells and the HCT116 cells expressing mutated KRAS.

First, for the PANC-1 cells, the MIA PaCa-2 cells, or the HCT116 cells, GST-π siRNA and P21 siRNA each at a final concentration of 25 nM, or GST-π siRNA or P21 siRNA at a final concentration of 25 nM (all added with control siRNA at a final concentration of 25 nM) were added to each of the Petri dishes, while for the A549 cells, GST-π siRNA and P21 siRNA each at a final concentration of 50 nM, or GST-π siRNA or P21 siRNA at a final concentration of 50 nM (added with control siRNA at a final concentration of 50 nM) were added to each of the Petri dishes. For a control, control siRNA was added at a final concentration of 50 nM for the PANC-1 cells, the MIA PaCa-2 cells, or the HCT116 cells and at a final concentration of 100 nM for the A549 cells. After 2 days and after 4 days, the medium was replaced (RPMI 1640 supplemented with 10% FBS for the PANC-1 cells, DMEM supplemented with 10% FBS for the A549 cells and the MIA PaCa-2 cells, and McCoy supplemented with 10% FBS for the HCT116 cells). Again, GST-π siRNA or P21 siRNA was added at a final concentration of 25 nM (all added with control siRNA at a final concentration of 25 nM) for the PANC-1 cells, the MIA PaCa-2 cells, or the HCT116 cells, and at a final concentration of 50 nM (added with control siRNA at a final concentration of 50 nM) for the A549 cell to each of the Petri dishes. In this case as well, for a control, control siRNA was added at a final concentration of 50 nM for the PANC-1 cells or the MIA PaCa-2 cells and at a final concentration of 100 nM for the A549 cells. Then, the cells were cultured without the replacement of the medium. Seven days after the cell inoculation, the cells were dissociated and collected from the Petri dish by trypsin treatment, and the number of cells was counted. In this case, the phase difference images of the cells were also taken.

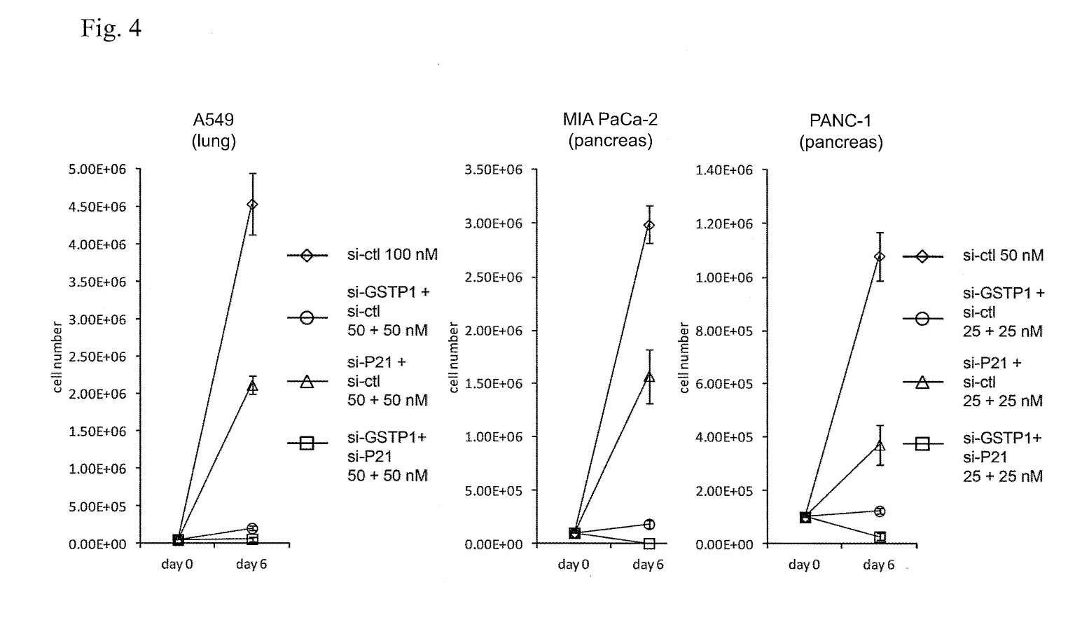

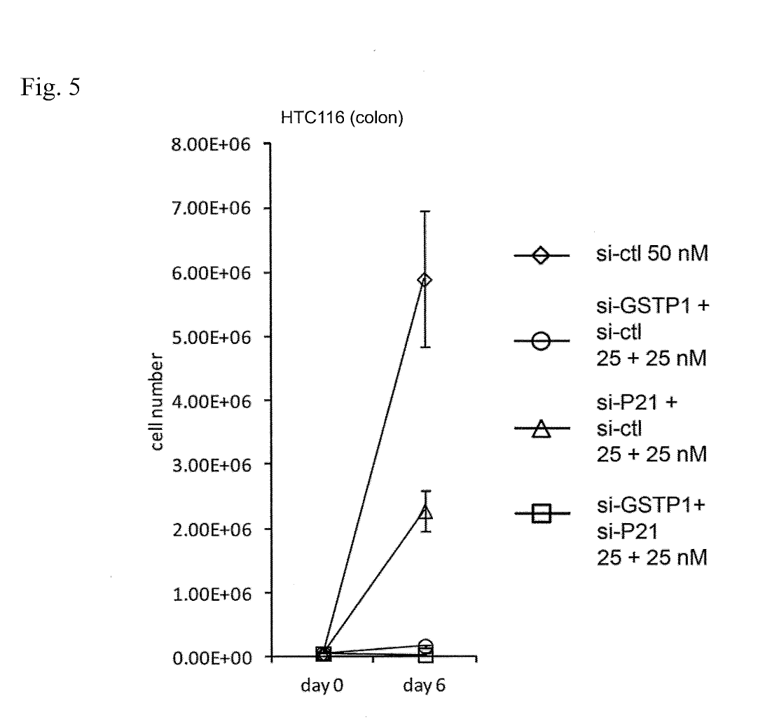

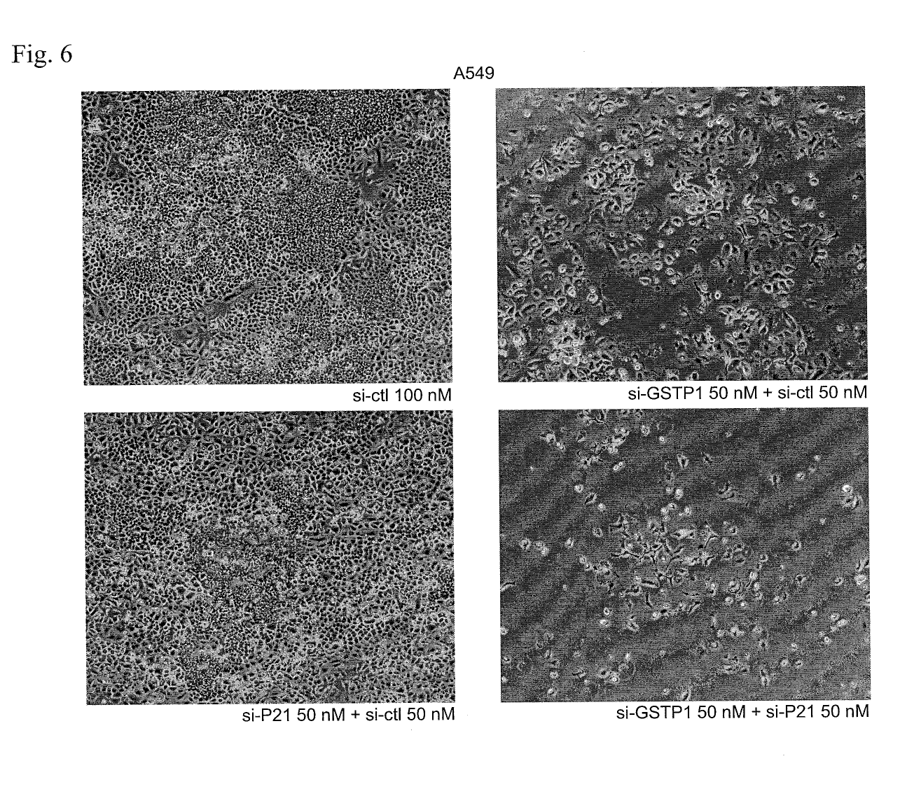

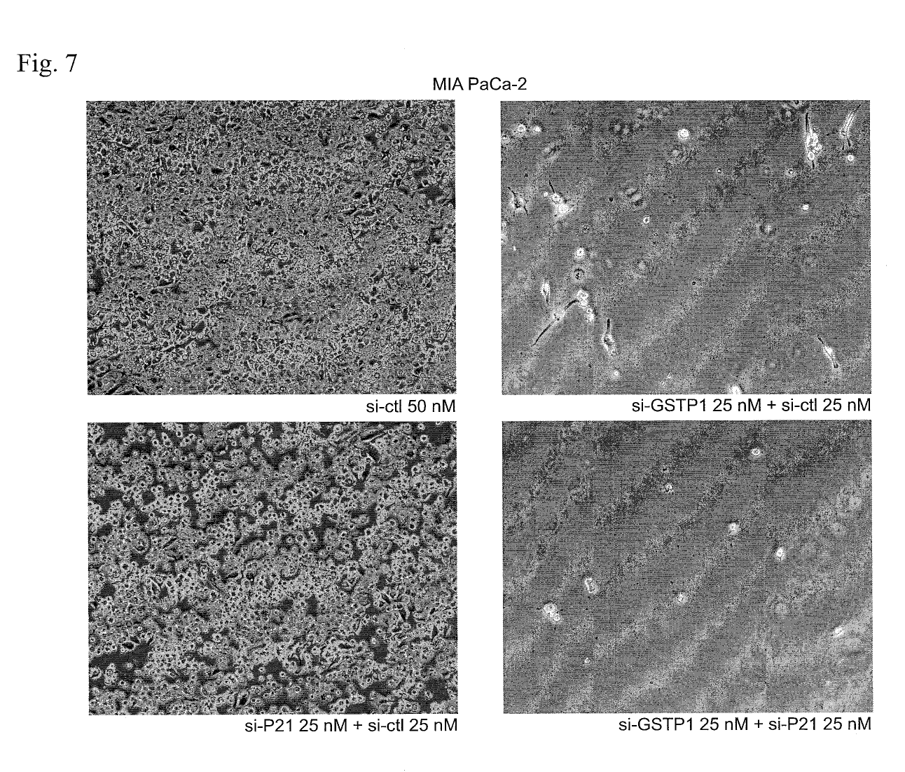

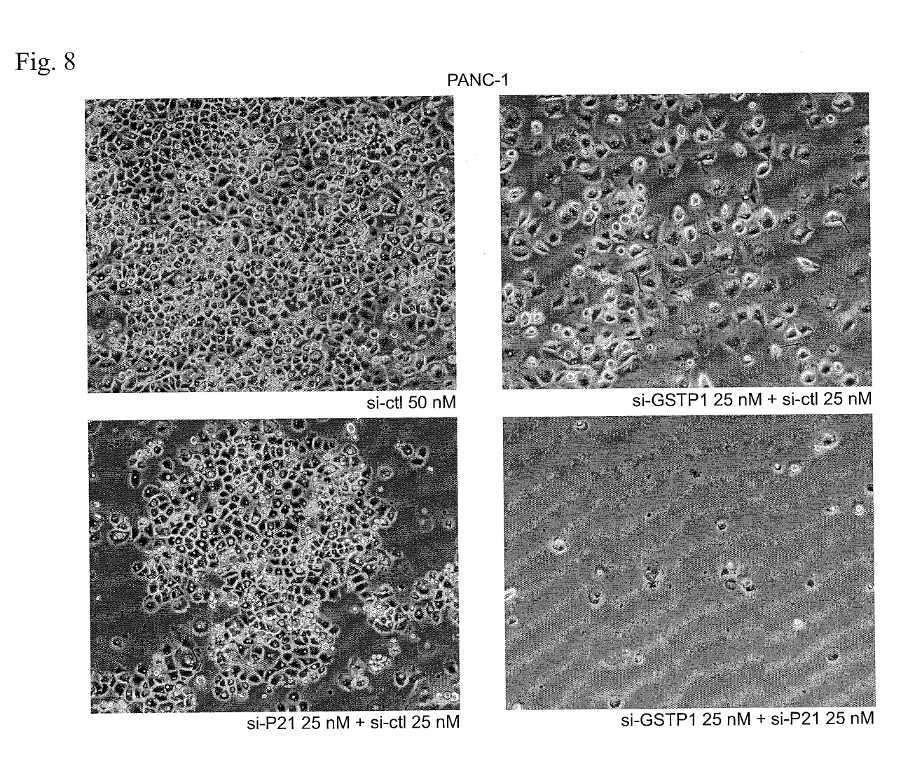

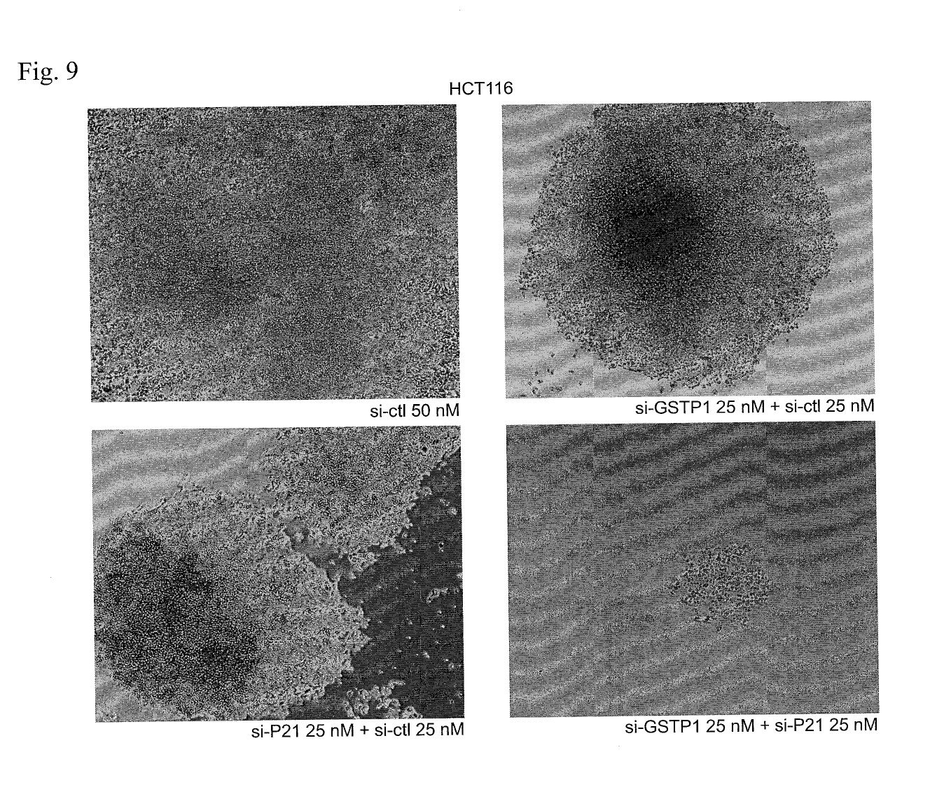

The results of measuring the number of cells for the A549 cells, the PANC-1 cells, and the MIA PaCa-2 cells are shown in FIG. 4. The results of measuring the number of cells for the HCT116 cells are shown in FIG. 5. Also, the phase difference image taken for the A549 cells is shown in FIG. 6. The phase difference image taken for the MIA PaCa-2 cells is shown in FIG. 7. The phase difference image taken for the PANC-1 cells is shown in FIG. 8. The phase difference image taken for the HCT116 cells is shown in FIG. 9.

As shown in FIGS. 4 and 5, when GST-π and P21 were both knocked down three times using GST-π siRNA and P21 siRNA, the number of the cancer cells (A549 cells, MIA PaCa-2 cells, PANC-1 cells, and HCT116 cells) expressing mutated KRAS is decreased 7 days after the cell inoculation with respect to the number of initially inoculated cells; thus it was revealed that cell death can be induced.

Moreover, as shown in FIGS. 6 to 9, the cells of each cell line (A549 cells, MIA PaCa-2 cells, PANC-1 cells, and HCT116 cells) expressing mutated KRAS in which GST-π was knocked down by GST-π siRNA became flat and large cells; thus it was able to be presumed that cell senescence was evoked. It was further revealed that when GST-π and P21 were both knocked down using GST-π siRNA and P21 siRNA, the cell senescence-like phenotype observed in the GST-π knockdown disappeared. From this result, it was considered that when GST-π and P21 were both knocked down using GST-π siRNA and P21 siRNA, the cell senescence evoked by the GST-π knockdown was inhibited by the P21 knockdown.

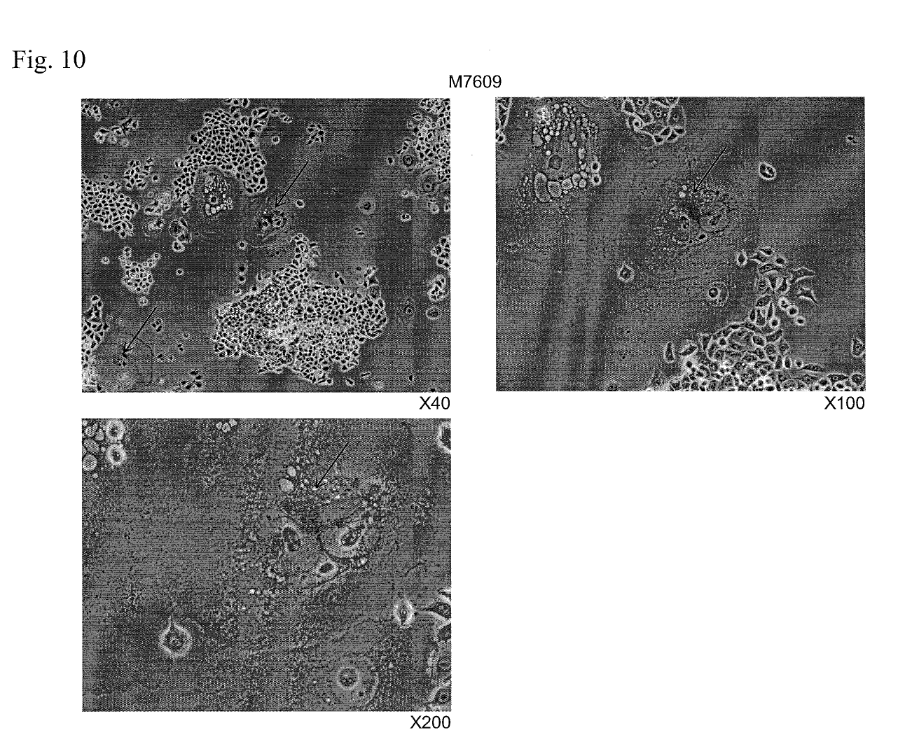

Whether the cell senescence as shown in FIGS. 6 to 9 was could be induced by knocking down GST-π was tested using the M7609 cells. First, GST-π siRNA was added at a final concentration of 30 nM to the Petri dish containing the M7609 cells. After 1 day and after 2 days, the medium was replaced (RPMI 1640 supplemented with 10% FBS). Again, GST-π siRNA was added at a final concentration of 30 nM to the Petri dish containing the M7609 cells. Then, the cells were cultured with the medium replaced every other day. Thirteen days after the cell inoculation, the cells were stained using Senescence (3-Galactosidase Staining Kit (Cell Signaling Technology, Inc.) according to the recommended protocol. A phase difference image was taken. The results are shown in FIG. 10. As shown in FIG. 10, the M7609 cells in which GST-π was knocked down became flat and large cells, and blue color development by (3-galactosidase was observed in the cells having such a phenotype; thus it is evident that cell senescence was evoked.

In addition, whether the cell death induced by knocking down both GST-π and P21 in the cancer cells expressing mutated KRAS was apoptosis was tested by measuring the expression level of an apoptosis-inducing factor PUMA gene.

First, GST-π siRNA and P21 siRNA each at a final concentration of 50 nM, or GST-π siRNA or P21 siRNA at a final concentration of 50 nM (both added with control siRNA at a final concentration of 50 nM) were added to each of the Petri dishes containing the A549 cells or the MIA PaCa-2 cells. For a control, control siRNA was added at a final concentration of 100 nM. After culture for 1 day without the replacement of the medium, the PUMA mRNA level was quantified by quantitative PCR using 7300 Real Time PCR System (Applied Biosystems, Inc.).

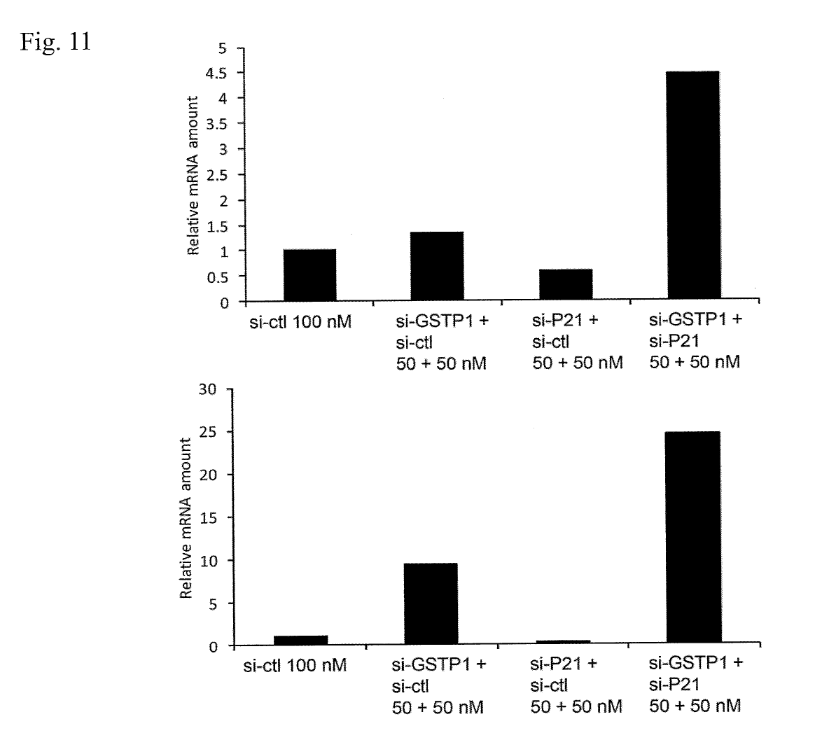

The results are shown in FIG. 11. As shown in FIG. 11, it was revealed that the mRNA level of the apoptosis-promoting factor PUMA is drastically increased by knocking down both GST-π and p21 using GST-π siRNA and P21 siRNA. From this result, it was revealed that the cell death induced by knocking down both GST-π and P21 is apoptosis.

Apoptosis-related protein groups are present in cells. The apoptosis-related proteins are broadly classified into two groups: an apoptosis-inhibiting protein group and an apoptosis-inducing protein group. The apoptosis-inhibiting protein group includes Bcl-2, Bcl-XL, Bcl-W, MCL-1, and Bcl-B. Also, the apoptosis-inducing protein group includes Bax, Bak, BOK, BIM, BID, BAD, NOXA, and PUMA. In general, the apoptosis-inhibiting proteins such as Bcl-2, Bcl-XL, and MCL-1 reside on mitochondrial outer membranes and inhibit the release of cytochrome C to inhibit apoptosis. On the other hand, the apoptosis-inducing protein groups such as Bax, BIM, BID, and BAD reside in cytoplasms, but translocate to mitochondrial outer membranes in response to death signals and promote the release of cytochrome C to induce apoptosis.

Also, upon activation due to DNA damage or the like, p53 promotes the transcription of Bax, NOXA, and PUMA to induce apoptosis. Particularly, PUMA is a protein that has been isolated as an apoptosis-inducing protein to be activated by p53. PUMA binds directly to Bcl-2, thereby inhibiting the apoptosis-inhibiting effect of Bcl-2 and inducing the apoptosis of the cell.

From these results of Experiment 1, it was shown that when a drug inhibiting GST-π and a drug inhibiting P21 are allowed to act on a cancer cell, cell growth can be drastically inhibited and further cell death can be strongly induced. Even if the drug inhibiting GST-π is allowed to act alone on a cancer cell, cell death cannot be induced, though cell growth can be inhibited. Even if the drug inhibiting P21 is allowed to act alone on the cell, cell growth can be inhibited merely slightly. Accordingly, it is a surprising effect that the cell death can be induced for a cancer cell by allowing both of these drugs to act thereon.

### [Experiment 2]

In Experiment 1, the synthetic lethality for cancer cells was demonstrated using GST-π siRNA and P21 siRNA. In this Experiment 2, a cell cycle-regulating protein that exhibited synthetic lethality by inhibition together with GST-π was screened for.

First, a MIA PaCa-2 cell suspension having a concentration of 1×10⁴ cells/mL was prepared with DMEM supplemented with 10% FBS and 1% L-glutamine, and this was inoculated at 100 μL/well to a 96-well plate and then cultured for 18 hours in DMEM supplemented with 10% FBS and 1% L-glutamine. The MIA PaCa-2 cells that became 20 to 30% confluent were transfected with GST-π siRNA-2 and/or siRNA against a target gene using Lipofectamine RNAi MAX as follows.

The Lipofectamine/siRNA mixed solution for transfection was prepared as follows: first, 51 μL of DNase free water (Ambion, Life Technologies Corp.) was added to 0.1 nmol of each siRNA contained in Human siGENOME siRNA Library—Cell Cycle Regulation—SMART pool (GE Healthcare Dharmacon Inc.) and left standing at room temperature for 90 minutes. An siRNA solution in which this aqueous siRNA solution was supplemented with 19.9 μL of OPTI-MEM was prepared (solution A). Next, a 50 μM aqueous GST-π siRNA-2 solution and a 50 μM aqueous control siRNA solution were each diluted with OPTI-MEM in ten times to prepare diluted solutions of 5 μM GST-π siRNA-2 and 5 μM control siRNA (solution B). 31.2 μL of the solution A and 8.8 μL of the solution B were mixed (solution C). Next, a Lipofectamine solution in which 150 μL of Lipofectamine RNAi MAX and 2.35 mL of OPTI-MEM were mixed was prepared (solution D). Next, 37.5 μL of the solution C and 37.5 μL of the solution D were mixed and left standing at room temperature for 15 minutes (solution E).

The solution E was added to each well culturing MIA-PaCa-2 cell of the 96-well plate at 10 μL/well. Separately, a 50 μM aqueous control siRNA solution (5.5 μL) and OPTI-MEM (189.5 μL) were mixed to prepare a solution (solution F). Next, a 50 μM aqueous control siRNA solution was diluted with OPTI-MEM in ten times to prepare 5 μM control siRNA (solution G). 31.2 μL of the solution F and 8.8 μL of the solution G were mixed (solution H). Next, a Lipofectamine solution in which 150 μL of Lipofectamine RNAi MAX and 2.35 mL of OPTI-MEM were mixed was prepared (solution I). Next, 37.5 μL of the solution H and 37.5 μL of the solution I were mixed and left standing at room temperature for 15 minutes (solution J). The solution J was added to each well culturing MIA-PaCa-2 cell of the 96-well plate at 10 μL/well. Then, the cells were cultured in DMEM supplemented with 10% FBS and 10% L-glutamine. After 5 days, a growth evaluation test was conducted using CyQUANT NF Cell Proliferation Assay Kit (Invitrogen Corp.).

First, 1×HBSS buffer was added to 22 μL of CyQUANT NF dye reagent to prepare a staining reaction solution for CyQUANT NF Cell proliferation Assay. The medium of the transfected cells mentioned above was aspirated, and 50 μL of the staining reaction solution was added thereto. The cells were left standing at 37° C. for 30 minutes. Then, a fluorescence wavelength of 520 nm was observed in excitation with an excitation wavelength of 480 nm.

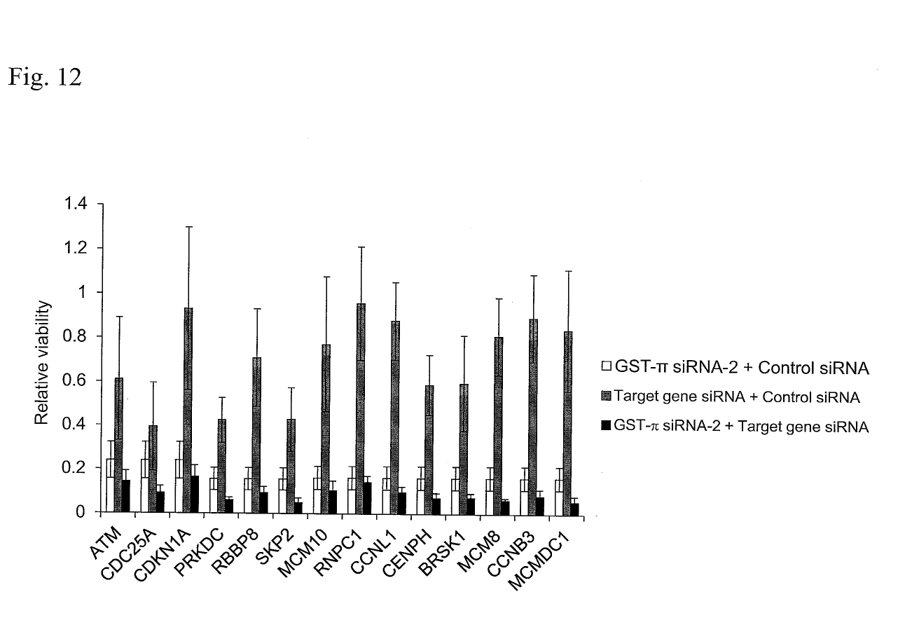

The results are shown in FIG. 12. As a result of screening 170 types of genes encoding cell cycle-regulating proteins in terms of synthetic lethality with GST-π, ATM, as shown in FIG. 12, CDC25A, PRKDC, RBBP8, SKP2, MCM10, RNPC1, CCNL1, CENPH, BRSK1, MCM8, CCNB3, and MCMDC1 in addition to P21 demonstrated in Experiment 1 were able to be screened for as cell cycle-regulating proteins that exhibited synthetic lethality by inhibition together with GST-π. Among them, P21, RNPC1, CCNL1, MCM8, CCNB3, and MCMDC1 were able to be screened for as cell cycle-regulating proteins that inhibited cell growth merely slightly (rate of growth inhibition: less than 20%) when inhibited alone, but exhibited synthetic lethality only when inhibited together with GST-π. Accordingly, it can be concluded that a drug inhibiting a cell cycle-regulating protein selected from P21, RNPC1, CCNL1, MCM8, CCNB3, and MCMDC1 is very low toxic in itself and is excellent in safety.

### [Experiment 3]

In Experiment 2, a cell cycle-regulating protein that exhibited synthetic lethality by inhibition together with GST-π was screened for. In this Experiment 3, a protein having an anti-apoptotic function that exhibited synthetic lethality by inhibition together with GST-π was screened for.

First, a MIA PaCa-2 cell suspension having a concentration of 1×10⁴ cells/mL was prepared with DMEM supplemented with 10% FBS and 1% L-glutamine, and this was inoculated at 100 μL/well to a 96-well plate and then cultured for 18 hours in DMEM supplemented with 10% FBS and 1% L-glutamine. The MIA PaCa-2 cells that became 20 to 30% confluent were transfected with GST-π siRNA-2 and/or siRNA against a target gene using Lipofectamine RNAi MAX as follows.

The Lipofectamine/siRNA mixed solution for transfection was prepared as follows: first, 51 μL of DNase free water (Ambion, Life Technologies Corp.) was added to 0.1 nmol of each siRNA contained in a custom-siRNA Library, which contains uniquely-selected 140 types of genes considered to have an anti-apoptosis function (siGENOME SMART pool Cherry-pick Library, GE Healthcare Dharmacon Inc.) and left standing at room temperature for 90 minutes. An siRNA solution in which this aqueous siRNA solution was supplemented with 19.9 μL of OPTI-MEM was prepared (solution A). Next, a 50 μM aqueous GST-π siRNA-2 solution and a 50 μM aqueous control siRNA solution were each diluted with OPTI-MEM to prepare solutions of 5 μM GST-π siRNA-2 and 5 μM control siRNA (solution B). 31.2 μL of the solution A and 8.8 μL of the solution B were mixed (solution C). Next, a Lipofectamine solution in which 150 μL of Lipofectamine RNAi MAX and 2.35 mL of OPTI-MEM were mixed was prepared (solution D). Next, 37.5 μL of the solution C and 37.5 μL of the solution D were mixed and left standing at room temperature for 15 minutes (solution E). The solution E was added to each well culturing MIA-PaCa-2 cell of the 96-well plate at 10 μL/well.

Separately, a 50 μM aqueous control siRNA solution (5.5 μL) and OPTI-MEM (189.5 μL) were mixed to prepare a solution (solution F). Next, a 50 μM aqueous control siRNA solution was diluted with OPTI-MEM in ten times to prepare 5 μM control siRNA (solution G). 31.2 μL of the solution F and 8.8 μL of the solution G were mixed (solution H). Next, a Lipofectamine solution in which 150 μL of Lipofectamine RNAi MAX and 2.35 mL of OPTI-MEM were mixed was prepared (solution I). Next, 37.5 μL of the solution H and 37.5 μL of the solution I were mixed and left standing at room temperature for 15 minutes (solution J). The solution J was added to each well culturing MIA-PaCa-2 cell of the 96-well plate at 10 μL/well. Then, the cells were cultured in DMEM supplemented with 10% FBS and 1% L-glutamine. After 5 days, a growth evaluation test was conducted using CyQUANT NF Cell Proliferation Assay Kit (Invitrogen Corp.).

First, 11 mL of 1×HBSS buffer was added to 22 μL of CyQUANT NF dye reagent to prepare a staining reaction solution for CyQUANT NF Cell proliferation Assay. The medium of the transfected cells mentioned above was aspirated, and 50 μL of the staining reaction solution was added thereto. The cells were left standing at 37° C. for 30 minutes. Then, a fluorescence wavelength of 520 nm was observed in excitation with an excitation wavelength of 480 nm.

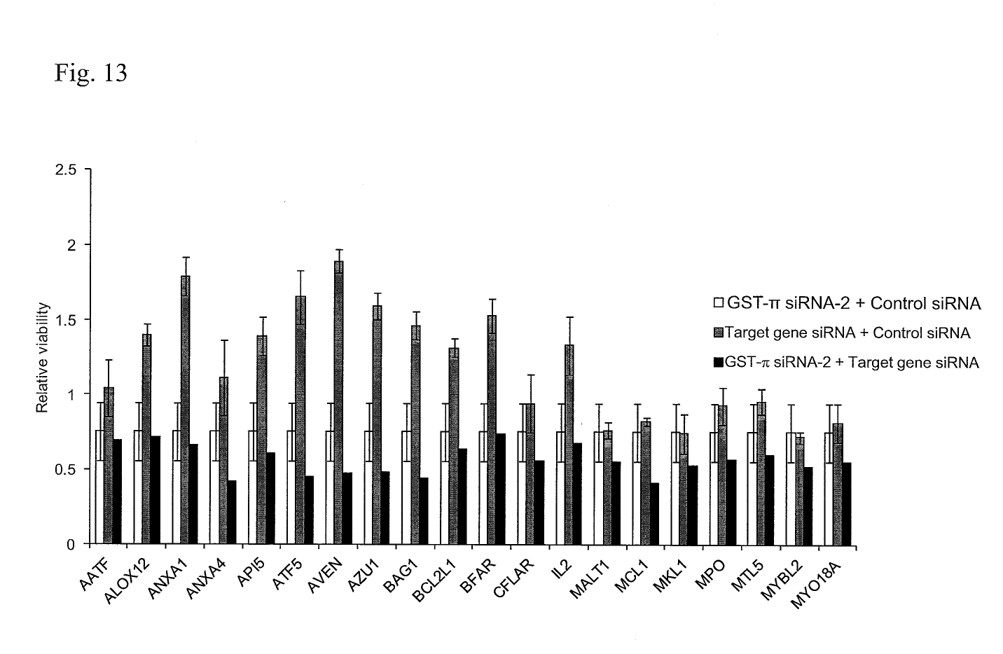

The results are shown in FIG. 13. As a result of screening 140 types of genes encoding anti-apoptosis-related proteins in terms of synthetic lethality with GST-π, as shown in FIG. 13, AATF, ALOX12, ANXA1, ANXA4, API5, ATF5, AVEN, AZU1, BAG1, BCL2L1, B FAR, CFLAR, IL2, MALT1, MCL1, MKL1, MPO, MTL5, MYB L2, and MYO18A were able to be screened for as anti-apoptosis-related proteins that exhibited synthetic lethality by inhibition together with GST-π.

### [Experiment 4]

In Experiment 2, a cell cycle-regulating protein that exhibited synthetic lethality by inhibition together with GST-π was screened for. In Experiment 3, a protein having an anti-apoptotic function that exhibited synthetic lethality by inhibition together with GST-π was screened for. In this Experiment 4, a PI3K signaling pathway-related protein that exhibited synthetic lethality by inhibition together with GST-π was screened for.

First, a MIA PaCa-2 cell suspension having a concentration of 1×10⁴ cells/mL was prepared with DMEM supplemented with 10% FBS and 1% L-glutamine, and this was inoculated at 100 μL/well to a 96-well plate and then cultured for 18 hours in DMEM supplemented with 10% FBS and 1% L-glutamine. The MIA PaCa-2 cells that became 20 to 30% confluent were transfected with GST-π siRNA-2 and/or siRNA against a target gene using Lipofectamine RNAi MAX as follows.

The Lipofectamine/siRNA mixed solution for transfection was prepared as follows: first, 51 μL of DNase free water (Ambion, Life Technologies Corp.) was added to 0.1 nmol of each siRNA contained in a custom-made siRNA library involving proprietary selected 80 types of genes thought to be involved in the PI3K signaling pathway (siGENOME SMARTpool Cherry-pick Library, GE Healthcare Dharmacon Inc.) and left standing at room temperature for 90 minutes. An siRNA solution in which this aqueous siRNA solution was supplemented with 19.9 μL of OPTI-MEM was prepared (solution A). Next, a 50 μM aqueous GST-π siRNA-2 solution and a 50 μM aqueous control siRNA solution were each diluted with OPTI-MEM in ten times to prepare diluted solutions of 5 μM GST-π siRNA-2 and 5 μM control siRNA (solution B). 31.2 μL of the solution A and 8.8 μL of the solution B were mixed (solution C). Next, a Lipofectamine solution in which 150 μL of Lipofectamine RNAi MAX and 2.35 mL of OPTI-MEM were mixed was prepared (solution D). Next, 37.5 μL of the solution C and 37.5 μL of the solution D were mixed and left standing at room temperature for 15 minutes (solution E). The solution E was added to each well culturing MIA-PaCa-2 cell of the 96-well plate at 10 μL/well.

Separately, a 50 μM aqueous control siRNA solution (5.5 μL) and OPTI-MEM (189.5 μL) were mixed to prepare a solution (solution F). Next, a 50 μM aqueous control siRNA solution was diluted with OPTI-MEM in ten times to prepare a diluted solution of 5 μM control siRNA (solution G). 31.2 μL of the solution F and 8.8 μL of the solution G were mixed (solution H). Next, a Lipofectamine solution in which 150 μL of Lipofectamine RNAi MAX and 2.35 mL of OPTI-MEM were mixed was prepared (solution I). Next, 37.5 μL of the solution H and 37.5 μL of the solution I were mixed and left standing at room temperature for 15 minutes (solution J). The solution J was added to each well culturing MIA-PaCa-2 cell of the 96-well plate at 10 μL/well to prepare a control. Then, the cells were cultured in DMEM supplemented with 10% FBS and 1% L-glutamine. After 5 days, a growth evaluation test was conducted using CyQUANT NF Cell Proliferation Assay Kit (Invitrogen Corp.).

First, 11 mL of 1×HBSS buffer was added to 22 μL of CyQUANT NF dye reagent to prepare a staining reaction solution for CyQUANT NF Cell proliferation Assay. The medium of the transfected cells mentioned above was aspirated, and 50 μL of the staining reaction solution was added thereto. The cells were left standing at 37° C. for 30 minutes. Then, a fluorescence wavelength of 520 nm was observed in excitation with an excitation wavelength of 480 nm.

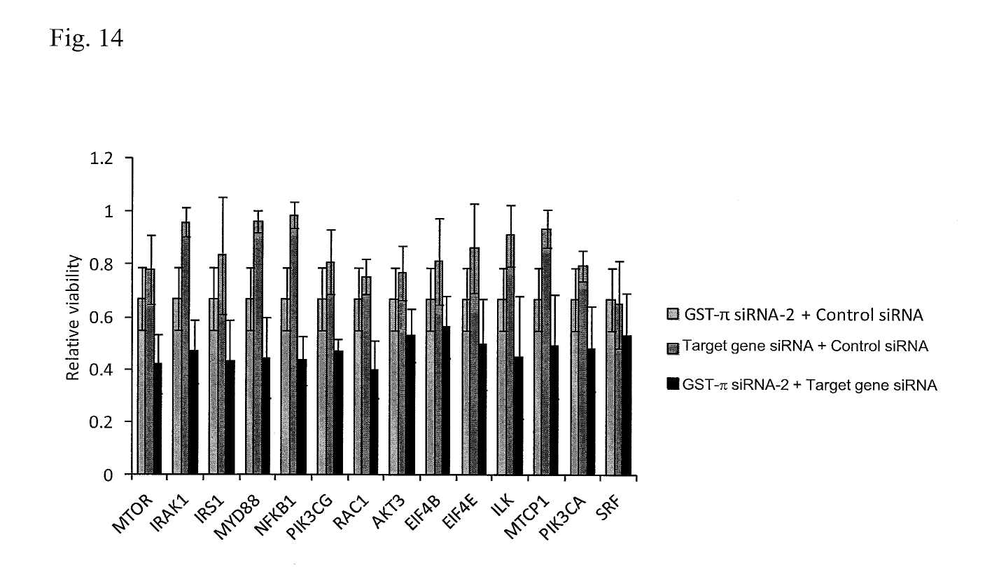

The results are shown in FIG. 14. As a result of screening 80 types of genes encoding PI3K signaling pathway-related proteins in terms of synthetic lethality with GST-π, as shown in FIG. 14, MTOR, IRAK1, IRS1, MYD88, NFKB1, PIK3CG, RAC1, AKT3, EIF4B, EIF4E, ILK, MTCP1, PIK3CA, and SRF were able to be screened for as PI3K signaling pathway-related proteins that exhibited synthetic lethality by inhibition together with GST-π. Among them, MTOR, IRAK1, IRS1, MYD88, NFKB1, PIK3CG, and RAC1 were found to exhibit a significantly high cell growth-inhibiting effect when inhibited together with GST-π.

### [Experiment 5]

In this Experiment 5, a cell cycle-regulating protein MYLK (HEPATOLOGY, Vol. 44, No. 1, 2006, 152-163) was studied for its synthetic lethality against A549 cells (human lung cancer cells having KRAS mutation) when inhibited together with GST-π.

First, one day before transfection with siRNA, 0.25×10⁵ cells/well of the A549 cells were inoculated, together with 2.25 mL of DMEM (antibiotic-free) supplemented with 10% FBS, to each well. In this experiment, a 6-well plate was used, and each sample was tested in triplicate. The cultured A549 cells were transfected with GST-π siRNA-3 and/or MYLK siRNA (two types (MYLKa and MYLKb) were used) using Lipofectamine RNAi MAX as follows.

The Lipofectamine/siRNA mixed solution for transfection was prepared as follows: first, an siRNA solution in which a 50 nM GST-π siRNA-3 solution and a 50 nM MYLK siRNA solution (0.5 μL each) were mixed and supplemented with 124 μL of OPTI-MEM was prepared. As for a control siRNA solution, siRNA solutions were also prepared as to the combination of the control siRNA solution and the GST-π siRNA-3 solution and the combination of the control siRNA solution and the MYLK siRNA solution so as to have the same siRNA concentrations. Also, a Lipofectamine solution in which 7.5 μL of Lipofectamine RNAi MAX and 117.5 μL of OPTI-MEM were mixed was prepared. Then, each prepared siRNA solution and the Lipofectamine solution were mixed and left standing at room temperature for 5 minutes.

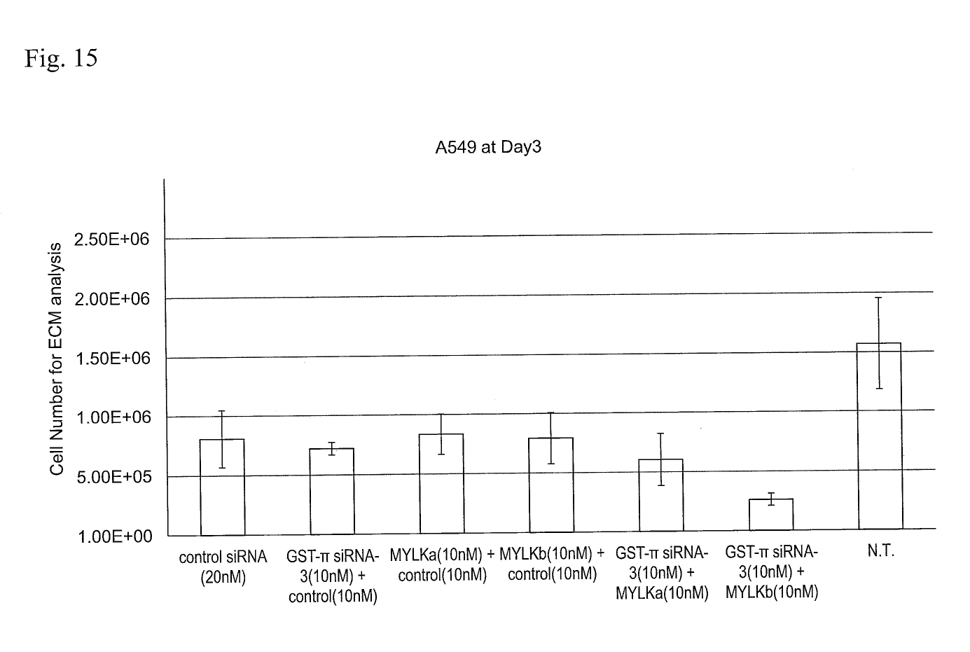

The obtained mixed solution was added dropwise to each well so that 2.5 mL thereof was finally contained in each well (final concentration of the siRNA: 20 nM). Then, the cells were cultured at 37° C. for 75 hours under 5% CO₂ conditions with mild shaking. After the completion of culture, a growth evaluation test was conducted in the same way as in Experiments 1 to 4. The results are shown in FIG. 15. As shown in FIG. 15, MYLK, a cell cycle-regulating protein, was found to exhibit synthetic lethality against cancer cells when inhibited together with GST-π. The results shown in FIG. 15 demonstrated that a GST-π-inhibiting drug and a MYLK-inhibiting drug exhibited a cell growth-inhibiting effect merely slightly when allowed to act each alone on cancer cells, whereas the GST-π-inhibiting drug and the MYLK-inhibiting drug were able to inhibit cell growth very strongly when allowed to act together on cancer cells.

siRNAs given below were used. In the description below, the upper-case letters represent RNAs, and the lower-case letters represent DNAs.

GST-π siRNA-3:
Sense strand:
(SEQ ID NO: 109)
CCUUUUGAGACCCUGCUGUtt
Antisense strand:
(SEQ ID NO: 110)
ACAGCAGGGUCUCAAAAGGtt
MYLKa:
Sense strand:
(SEQ ID NO: 111)
CUGGGGAAGAAGGUGAGUAtt
Antisense strand:
(SEQ ID NO: 112)
UACUCACCUUCUUCCCCAGtt
MYLKb:
Sense strand:
(SEQ ID NO: 113)
CAAGAUAGCCAGAGUUUAAtt
Antisense strand:
(SEQ ID NO: 114)
UUAAACUCUGGCUAUCUUGtt

1. A cell death-inducing agent for a cancer cell comprising, as active ingredients, a drug inhibiting GST-π and a drug inhibiting a homeostasis-related protein that exhibits synthetic lethality when inhibited together with GST-π.

2. A cell growth-inhibiting agent for a cancer cell comprising, as active ingredients, a drug inhibiting GST-π and a drug inhibiting a homeostasis-related protein that exhibits synthetic lethality when inhibited together with GST-π.

3. The agent according to claim 1, wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

4. The agent according to claim 3, wherein the cell cycle-regulating protein that exhibits synthetic lethality along with the inhibition of GST-π is at least one cell cycle-regulating protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

5. The agent according to claim 3, wherein the cell cycle-regulating protein that exhibits synthetic lethality along with the inhibition of GST-π is at least one protein selected from the group consisting of p21, RNPC1, CCNL1, MCM8, CCNB3, and MCMDC1.

6. The agent according to claim 3, wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

7. The agent according to claim 3, wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.

8. The agent according to claim 1, wherein the drug is a substance selected from the group consisting of an RNAi molecule, a ribozyme, an antisense nucleic acid, a DNA/RNA chimeric polynucleotide, and a vector for expressing at least one of them.

9. The agent according to claim 1, wherein the drug inhibiting a homeostasis-related protein is a compound that acts on the homeostasis-related protein.

10. The agent according to claim 1, wherein the agent induces apoptosis.

11. The agent according to claim 1 or 2, wherein the cancer cell is a cancer cell highly expressing GST-π.

12. A pharmaceutical composition for the treatment of a disease caused by abnormal cell growth, comprising an agent according to claim 1.

13. The pharmaceutical composition according to claim 12, wherein the disease is a cancer.

14. The pharmaceutical composition according to claim 13, wherein the cancer is a cancer highly expressing GST-π.

15. A method for screening for a cell death-inducing agent and/or a cell growth-inhibiting agent for a cancer cell that is used together with a drug inhibiting GST-π, comprising a step of selecting a drug inhibiting a homeostasis-related protein that exhibits synthetic lethality when inhibited together with GST-π.

16. The screening method according to claim 15, comprising the steps of: contacting a test substance with a cancer cell; measuring the expression level of the homeostasis-related protein in the cell; and selecting the test substance as a drug inhibiting the homeostasis-related protein when the expression level is decreased compared with that measured in the absence of the test substance.

17. The screening method according to claim 15, wherein the homeostasis-related protein that exhibits synthetic lethality along with the inhibition of GST-π is a protein selected from the group consisting of a cell cycle-regulating protein, an anti-apoptosis-related protein, and a PI3K signaling pathway-related protein.

18. The screening method according to claim 17, wherein the cell cycle-regulating protein that exhibits synthetic lethality along with the inhibition of GST-π is at least one cell cycle-regulating protein selected from the group consisting of ATM, CDC25A, p21, PRKDC, RBBP8, SKP2, MCM10, RNPC1, CCNL1, CENPH, BRSK1, MCM8, CCNB3, MCMDC1 and MYLK.

19. The screening method according to claim 17, wherein the cell cycle-regulating protein that exhibits synthetic lethality along with the inhibition of GST-π is at least one protein selected from the group consisting of p21, RNPC1, CCNL1, MCM8, CCNB3, and MCMDC1.

20. The screening method according to claim 17, wherein the anti-apoptosis-related protein that exhibits synthetic lethality along with the inhibition of GST-π is at least one anti-apoptosis-related protein selected from the group consisting of AATF, ALOX12, ANXA1, ANXA4, API5, ATF5, AVEN, AZU1, BAG1, BCL2L1, BFAR, CFLAR, IL2, MALT1, MCL1, MKL1, MPO, MTL5, MYBL2, and MYO18A.

21. The screening method according to claim 16, wherein the PI3K signaling pathway-related protein that exhibits synthetic lethality along with the inhibition of GST-π is at least one PI3K signaling pathway-related protein selected from the group consisting of MTOR, IRAK1, IRS1, MYD88, NFKB1, PIK3CG, RAC1, AKT3, EIF4B, EIF4E, ILK, MTCP1, PIK3CA, and SRF.
